(19) United States
(12) Patent Application Publication
(10) Pub. No.: US 2022/0273779 A1
(43) Pub. Date: Sep. 1, 2022

(54) TAU PEPTIDE ANTIGENS AND ANTIBODIES BINDING THERETO FOR THE TREATMENT OF TAUOPATHIES

(71) Applicant: The Board of Regents of the University of Texas System, Austin, TX (US)

(72) Inventors: Marc I. DIAMOND, Dallas, TX (US); Kenneth DROMBOSKY, Dallas, TX (US); Dailu CHEN, Dallas, TX (US); Victor MANON, Dallas, TX (US); Xiaohua LIU, Dallas, TX (US); Hamid MIRZAEI, Dallas, TX (US); Jaime VAQUER-ALICEA, Dallas, TX (US); Hilda MIRBAHA, Dallas, TX (US); Lukasz A. JOACHIMIAK, Dallas, TX (US)

(73) Assignee: The Board of Regents of the University of Texas System, Austin, TX (US)

(21) Appl. No.: 17/295,152

(22) PCT Filed: Nov. 18, 2019
(86) PCT No.: PCT/US2019/061928

§ 371 Date: May 19, 2021

Related U.S. Application Data
(60) Provisional application No. 62/769,281, filed on Nov. 19, 2018.

(51) Int. Cl. A61K 39/00; A61P 25/28; A61K 38/16; A61K 38/03; A61K 9/00

(52) U.S. Cl. CPC A61K 39/0007; A61P 25/28; A61K 38/16; A61K 38/03; A61K 9/0019; A61K 9/0073; A61K 2039/6081

(57) ABSTRACT

The disclosure provides methods and compositions for treating and diagnosing tauopathies. More specifically, the disclosure relates to the identification of epitopes on tau and their use as vaccines or as reagents to generate monoclonal antibodies that can be used for both diagnosis and treatment of tau-related diseases.

### PRIORITY CLAIM

This application is a national phase application under 35 U.S.C. § 371 of International Application No. PCT/US2019/061928, filed Nov. 18, 2019, which claims benefit of priority to U.S. Provisional Application Ser. No. 62/769,281, filed Nov. 19, 2018, the entire contents of each of which are hereby incorporated by reference.

### BACKGROUND

### 1. Field

This disclosure relates to the fields of medicine and cell biology. More specifically, the disclosure is directed to peptide antigens and antibodies thereto for the treatment and diagnosis of tauopathies such as Alzheimer's Disease.

### 2. Related Art

A wide variety of neurodegenerative disorders involve accumulation of insoluble tau protein. These disorders are collectively known as tauopathies due to the presence of tau tangles though their clinical manifestation vary widely. One well-known tauopathy, Alzheimer's disease (AD) is defined neuropathologically by the accumulation of beta-amyloid plaques and neurofibrillary tangles (NFT) containing tau protein. NFTs in AD have been found to consist of hyperphosphorylated forms of the tau protein. Hyperphosphorylated tau exhibits reduced ability to bind and stabilize microtubules and can self-aggregate to form insoluble paired helical filaments (PHFs), which comprise NFTs (Gustke et al., 1992; Bramblett et al., 1993; Alonso et al., 1996). The incidence of NFTs is positively correlated with cognitive deficit and neuronal loss in AD (Arriagada et al., 1992; Gomez-Isla et al., 1997), and the discovery that mutations in the tau gene underlie autosomal dominant forms of frontotemporal dementia suggests that pathological changes in tau can serve as a principal cause of neurodegeneration and cognitive impairment (Hutton et al., 1998; Poorkaj et al., 1998; Spillantini et al., 1998). In view of this, tau phosphorylation has been studied as a possible mediator of tauopathies. However, new evidence suggests that phosphorylation alone is not sufficient to drive tauopathies, and that distinct pathological conformations must be considered. In particular, tau appears to transition from an inert state, with more collapsed local structure to a more extended form that exposes new epitopes (Mirbaha et al., 2018; Chen et al., 2019).

### SUMMARY

Thus, in accordance with the present disclosure, there is provided a method of treating a subject with or at risk of developing a tauopathy comprising administering to said subject one or more peptides from Tables B or C. The method may further comprise administering the same or a different peptide from Tables B or C at a second time point. The subject may suffer from Alzheimer's Disease, Progressive Supranuclear Palsy, Corticobasal Degeneration, Frontotemporal Dementia, Multiple Sclerosis, Argyrophilic Grain Disease, Chronic Traumatic Encephalopathy, Subacute Sclerosing Panencephalitis or Primary Age-Related Tauopathy. The method may further comprise administering to said subject an adjuvant and/or a biological response modifier.

The subject may have been diagnosed with a tauopathy. The subject may be a human subject or non-human mammalian subject. Administering may comprise intramuscular injection, oral delivery, subcutaneous injection, transdermal delivery, inhalation, intravenous injection, intrathecal or intraventricular injection. The peptide may be no more than about 100 residues in length, no more than about 50 residues in length, or no more than about 38 residues in length. The peptide may be at least 5 residues in length, is at least 6 residues in length, or is at least 10 residues in length.

In another embodiment, there is provided a method of treating a subject with or at risk of developing a tauopathy comprising administering to said subject one or more antibodies that bind to a tau epitope defined by one or more peptides from from Tables B or C. The method may further comprise administering the same or a different antibody that binds to a tau epitope defined by one or more peptides from from Tables B or C. The subject may suffer from Alzheimer's Disease, Progressive Supranuclear Palsy, Corticobasal Degeneration, Frontotemporal Dementia, Multiple Sclerosis, Argyrophilic Grain Disease, Chronic Traumatic Encephalopathy, or Primary Age-Related Tauopathy. The method may further comprise administering to said subject an adjuvant and/or a biological response modifier.

The subject may have been diagnosed with a tauopathy. The subject may be a human subject or non-human mammalian subject. Administering may comprise intramuscular injection, oral delivery, subcutaneous injection, transdermal delivery, inhalation, intravenous injection, intrathecal or intraventricular injection. The antibody fragment may be a recombinant scFv (single chain fragment variable) antibody, Fab fragment, F(ab′)₂ fragment, or Fv fragment, or said antibody is a chimeric antibody or a bispecific antibody. The antibody may be an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern.

Also provided is a peptide vaccine comprising one or more peptides from Tables B or C in a pharmaceutically acceptable diluent, buffer or excipient. The vaccine may further comprise an adjuvant and/or a biological response modifier. The vaccine may be formulated for intramuscular injection, oral delivery, subcutaneous injection, transdermal delivery, inhalation, intravenous injection, intrathecal or intraventricular injection. The peptide may be no more than about 100 residues in length, no more than about 50 residues in length, or no more than about 37 residues in length. The peptide may be at least 5 residues in length, is at least 10 residues in length, or is at least 16 residues in length.

In another embodiment, there is provided a vaccine comprising one or more antibodies that bind to a tau epitope defined by one or more peptides from from Tables B or C. The vaccine may further comprise an adjuvant and/or a biological response modifier. The vaccine may be formulated for intramuscular injection, oral delivery, subcutaneous injection, transdermal delivery, inhalation, intravenous injection, intramuscular injection, intrathecal or intraventricular injection. The antibody fragment may be a recombinant scFv (single chain fragment variable) antibody, Fab fragment, F(ab′)₂ fragment, or Fv fragment, or said antibody is a chimeric antibody or a bispecific antibody. The antibody may be an IgG, or a recombinant IgG antibody or antibody fragment comprising an Fc portion mutated to alter (eliminate or enhance) FcR interactions, to increase half-life and/or increase therapeutic efficacy, such as a LALA, N297, GASD/ALIE, YTE or LS mutation or glycan modified to alter (eliminate or enhance) FcR interactions such as enzymatic or chemical addition or removal of glycans or expression in a cell line engineered with a defined glycosylating pattern.

In still yet another embodiment, there is provided a method of detecting tau protein or a structurally related antigen in a subject comprising (a) contacting a sample from said subject with one or more antibodies or antibody fragments that bind to a tau epitope defined by one or more peptides from from Tables B or C; and (b) detecting said tau protein or structurally related antigen in said sample by binding of said one or more antibodies or antibody fragments to said tau protein or structurally related antigen in said sample. The sample may be a body fluid, such as blood, cerebrospinal fluid, sputum, tears, saliva, mucous, serum, semen, cervical or vaginal secretions, amniotic fluid, placental tissues, urine, exudate, transudate, tissue scrapings or feces, or may be a tissue, such as brain tissue.

Detection may comprise ELISA, RIA, lateral flow assay, Western blot or immunohistochemistry. The method may further comprise performing steps (a) and (b) a second time and determining a change in tau or structurally related antigen levels as compared to the first assay. The antibody fragment may be a recombinant scFv (single chain fragment variable) antibody, Fab fragment, F(ab′)₂ fragment, or Fv fragment. The subject may be suspected of having a tauopathy or may have been previously diagnosed with a tauopathy. The subject may be a human or non-human mammal. The tauopathy may be Alzheimer's Disease, Progressive Supranuclear Palsy, Corticobasal Degeneration, Frontotemporal Dementia, Multiple Sclerosis, Argyrophilic Grain Disease, Chronic Traumatic Encephalopathy, or Primary Age-Related Tauopathy.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE FIGURES

The following drawings form part of the present specification and are included to further demonstrate certain aspects of the present disclosure. The disclosure may be better understood by reference to one or more of these drawings in combination with the detailed description.

FIGS. 1A-D. Tauopathy mutations drive aggregation propensity. (FIG. 1A) Schematic of tau-RD and the derived peptides representing minimal structural elements around ³⁰⁶VQIVYK³¹¹ (SEQ ID NO: 1). (FIG. 1B) ThT aggregation curves for VQIVYK (SEQ ID NO: 1) and DNIKHV (SEQ ID NO: 2) hexapeptides. (FIG. 1C) Primary amino acid sequence mapped onto a cartoon of the predicted minimal R2R3 structural element proximal to ³⁰⁶VQIVYK³¹¹ (SEQ ID NO: 1). Mutations are shown as spheres. (FIG. 1D) ThT aggregation curves for R2R3 wild type and mutant peptides.

FIGS. 2A-B. Enhancing β-hairpin structure rescues spontaneous aggregation phenotypes. (FIG. 2A) Schematic of proline and fluorinated proline analogs used to generate cis and trans proline conformers at the position corresponding to P301 (red ball) in peptide models. (FIG. 2B) ThT aggregation reactions of the cis, trans, and neutral (Ea) proline analogs substituted into the R2R3 peptide (200 μM). ThT signals are an average of at least 6 independent experiments.

FIGS. 3A-B. Regions in tau-RD suitable to target for novel therapeutics and diagnostics. (FIG. 3A) Schematic of tau-RD and the derived peptides representing minimal structural elements spanning all interrepeat regions including R1R2, R3R4, R4R′ and the splice variant R1R3. (FIG. 3B) Location of the P301 R2R3 equivalent proline in each interrepeat element. Trans-proline configurations of these elements are predicted to represent strong therapeutic and diagnostic elements. (SEQ ID NOS: 146, 147, 148, 149 and 121; top to bottom).

FIG. 4. Sample data from seeding assays with IP and supernatant. Graph comparing the seeding activity of the IP and supernatant fractions resulting from immunoprecipitation of 15 μg of total protein from brain homogenates of 4 different tauopathies with antibodies from 100 μl of conditioned media from an anti-R1R2transP270 hybridoma. All results are normalized to the pre-IP seeding activity of 15 μg of protein from the indicated brain homogenate.

### DESCRIPTION OF ILLUSTRATIVE EMBODIMENTS

Tau proteins (or τ proteins, after the Greek letter with that name) are proteins that stabilize microtubules. They are abundant in neurons of the central nervous system and are less common elsewhere but are also expressed at very low levels in CNS astrocytes and oligodendrocytes. Pathologies and dementias of the nervous system such as Alzheimer's disease and Parkinson's disease are associated with tau proteins that have become defective and no longer stabilize microtubules properly.

The tau proteins are the product of alternative splicing from a single gene that in humans is designated MAPT (microtubule-associated protein tau) and is located on chromosome 17. The tau proteins were identified in 1975 as heat-stable proteins essential for microtubule assembly and since then, they have been characterized as intrinsically disordered proteins.

Tau protein is a highly soluble microtubule-associated protein (MAP). In humans, these proteins are found mostly in neurons compared to non-neuronal cells. One of tau's main functions is to modulate the stability of axonal microtubules. Other nervous system MAPs may perform similar functions, as suggested by tau knock out mice that did not show abnormalities in brain development—possibly because of compensation in tau deficiency by other MAPs. Tau is not present in dendrites and is active primarily in the distal portions of axons where it provides microtubule stabilization but also flexibility as needed. This contrasts with MAP6 (STOP) proteins in the proximal portions of axons, which, in essence, lock down the microtubules and MAP2 that stabilizes microtubules in dendrites.

Tau proteins interact with tubulin to stabilize microtubules and promote tubulin assembly into microtubules. Tau has two ways of controlling microtubule stability: isoforms and phosphorylation.

Six tau isoforms exist in human brain tissue, and they are distinguished by their number of binding domains. Three isoforms have three binding domains and the other three have four binding domains. The binding domains are located in the carboxy-terminus of the protein and are positively charged (allowing it to bind to the negatively charged microtubule). The isoforms with four binding domains are better at stabilizing microtubules than those with three binding domains. The isoforms are a result of alternative splicing in exons 2, 3, and 10 of the tau gene.

Tau is a phosphoprotein with 79 potential Serine (Ser) and Threonine (Thr) phosphorylation sites on the longest tau isoform. Phosphorylation has been reported on approximately 30 of these sites in normal tau proteins. Phosphorylation of tau is regulated by a host of kinases, including PKN, a serine/threonine kinase. When PKN is activated, it phosphorylates tau, resulting in disruption of microtubule organization.

Phosphorylation of tau is also developmentally regulated. For example, fetal tau is more highly phosphorylated in the embryonic CNS than adult tau. The degree of phosphorylation in all six isoforms decreases with age due to the activation of phosphatases. Like kinases, phosphatases too play a role in regulating the phosphorylation of tau. For example, PP2A and PP2B are both present in human brain tissue and have the ability to dephosphorylate Ser396. The binding of these phosphatases to tau affects tau's association with MTs. Hyperphosphorylation of the tau protein (tau inclusions, pTau) can result in the self-assembly of tangles of paired helical filaments and straight filaments, which are involved in the pathogenesis of Alzheimer's disease, frontotemporal dementia, and other tauopathies.

All of the six tau isoforms are present in an often hyperphosphorylated state in paired helical filaments from Alzheimer's disease brain. In other neurodegenerative diseases, including Alzheimer's disease, the deposition of aggregates enriched in certain tau isoforms has been reported, which defines them collectively as “tauopathies.” When misfolded, this otherwise very soluble protein can form extremely insoluble aggregates that contribute to a number of neurodegenerative diseases. Tau protein has a direct effect on the breakdown of a living cell caused by tangles that form and block nerve synapses. Tangles are clumps of tau protein that stick together and block essential nutrients that need to be distributed to cells in the brain, causing the cells to die. Some aspects of how the disease functions also suggest that it has some similarities to prion proteins, in that pathogenic forms of the tau protein can exit cells and spread pathology between cells of the brain in a progressive fashion, by acting as conformational templates.

The inventor developed a method to identify epitopes on the tau protein based on combining immunoprecipitation and a cell-based biosensor assay that reports on the presence of tau “prions,” which are pathogenic seeds present in brain tissue. Using this approach, they identified epitopes on tau that, when targeted by appropriate antibodies, will preferentially immunoprecipitate pathogenic tau prions vs. “native” (i.e., non-pathogenic) tau. These epitopes could be useful as active vaccines (containing one or more epitopes) to produce an immune response that targets tau in patients, or to produce monoclonal antibodies for use as passive vaccines in patients with neurodegenerative diseases caused by tau accumulation.

This and other aspects of the disclosure are set forth in detail below.

### I. TAUOPATHIES AND TAU PROTEIN

Tauopathies are a class of neurodegenerative diseases associated with the pathological aggregation of tau protein in neurofibrillary or gliofibrillary tangles in the human brain. Tangles are formed by hyperphosphorylation of a microtubule-associated protein known as tau, causing the protein to dissociate from microtubules and form aggregates in an insoluble form. (These aggregations of hyperphosphorylated tau protein are also referred to as paired helical filaments). The precise mechanism of tangle formation is not completely understood, and it is still controversial as to whether tangles are a primary causative factor in the disease or play a more peripheral role.

A. Alzheimer's Disease

AD is a progressive, neurodegenerative disease characterized by memory loss, language deterioration, impaired visuospatial skills, poor judgment, indifferent attitude, but preserved motor function. AD usually begins after age 65, however, its onset may occur as early as age 40, appearing first as memory decline and, over several years, destroying cognition, personality, and ability to function. Confusion and restlessness may also occur. The type, severity, sequence, and progression of mental changes vary widely. The early symptoms of AD, which include forgetfulness and loss of concentration, can be missed easily because they resemble natural signs of aging. Similar symptoms can also result from fatigue, grief, depression, illness, vision or hearing loss, the use of alcohol or certain medications, or simply the burden of too many details to remember at once.

There is no cure for AD and no way to slow the progression of the disease. For some people in the early or middle stages of the disease, medication such as tacrine may alleviate some cognitive symptoms. Aricept (donepezil), Razadyne (galantamine), and Exelon (rivastigmine) are reversible acetylcholinesterase inhibitors that are indicated for the treatment of mild to moderate dementia of the Alzheimer's type. Also, some medications may help control behavioral symptoms such as sleeplessness, agitation, wandering, anxiety, and depression. These treatments are aimed at making the patient more comfortable.

AD is a progressive disease. The course of the disease varies from person to person. Some people have the disease only for the last 5 years of life, while others may have it for as many as 20 years. The most common cause of death in AD patients is infection.

The molecular aspect of AD is complicated and not yet fully defined. As stated above, AD is characterized by the formation of amyloid plaques and neurofibrillary tangles in the brain, particularly in the hippocampus which is the center for memory processing. Several molecules contribute to these structures: amyloid β protein (Aβ), presenilin (PS), cholesterol, apolipoprotein E (ApoE), and Tau protein. Of these, Aβ appears to play the central role.

Aβ contains approximately 40 amino acid residues. The 42 and 43 residue forms are much more toxic than the 40-residue form. Aβ is generated from an amyloid precursor protein (APP) by sequential proteolysis. One of the enzymes lacks sequence specificity and thus can generate Aβ of varying (39-43) lengths. The toxic forms of Aβ cause abnormal events such as apoptosis, free radical formation, aggregation and inflammation. Presenilin encodes one of the two proteases responsible for cleaving APP into Aβ. There are two forms—PS1 and PS2. Mutations in PS1, causing production of Aβ₄₂, are the typical cause of early onset AD.

Cholesterol-reducing agents have been alleged to have AD-preventative capabilities, although no definitive evidence has linked elevated cholesterol to increased risk of AD. However, the discovery that Aβ contains a sphingolipid binding domain lends further credence to this theory. Similarly, ApoE, which is involved in the redistribution of cholesterol, is now believed to contribute to AD development. As discussed above, individuals having the ApoE4 allele, which exhibits the least degree of cholesterol efflux from neurons, are more likely to develop AD.

Tau protein, associated with microtubules in normal brain, forms paired helical filaments (PHFs) in AD-affected brains which are the primary constituent of neurofibrillary tangles. Recent evidence suggests that Aβ proteins may cause hyperphosphorylation of Tau proteins, leading to disassociation from microtubules and aggregation into PHFs.

It is well-established that the neurodegeneration of AD involves dysfunction of neuronal mitochondria (Carvalho et al., 2015); ameliorating this dysfunction with A₃AR agonist treatment will thus be a potential therapeutic approach for AD.

B. Multiple Sclerosis

Multiple sclerosis (MS) is a demyelinating disease in which the insulating covers of nerve cells in the brain and spinal cord are damaged. This damage disrupts the ability of parts of the nervous system to communicate, resulting in a range of signs and symptoms, including physical, mental, and sometimes psychiatric problems. Specific symptoms can include double vision, blindness in one eye, muscle weakness, trouble with sensation, or trouble with coordination. MS takes several forms, with new symptoms either occurring in isolated attacks (relapsing forms) or building up over time (progressive forms). Between attacks, symptoms may disappear completely; however, permanent neurological problems often remain, especially as the disease advances.

While the cause is not clear, the underlying mechanism is thought to be either destruction by the immune system or failure of the myelin-producing cells. Proposed causes for this include genetics and environmental factors such as being triggered by a viral infection. MS is usually diagnosed based on the presenting signs and symptoms and the results of supporting medical tests.

There is no known cure for multiple sclerosis. Treatments attempt to improve function after an attack and prevent new attacks. Medications used to treat MS, while modestly effective, can have side effects and be poorly tolerated. Physical therapy can help with people's ability to function. Many people pursue alternative treatments, despite a lack of evidence of benefit. The long-term outcome is difficult to predict, with good outcomes more often seen in women, those who develop the disease early in life, those with a relapsing course, and those who initially experienced few attacks. Life expectancy is on average 5 to 10 years lower than that of an unaffected population.

Multiple sclerosis is the most common immune-mediated disorder affecting the central nervous system. In 2015, about 2.3 million people were affected globally with rates varying widely in different regions and among different populations. That year about 18,900 people died from MS, up from 12,000 in 1990. The disease usually begins between the ages of 20 and 50 and is twice as common in women as in men. MS was first described in 1868 by Jean-Martin Charcot. The name multiple sclerosis refers to the numerous scars (sclerae—better known as plaques or lesions) that develop on the white matter of the brain and spinal cord. A number of new treatments and diagnostic methods are under development.

A person with MS can have almost any neurological symptom or sign, with autonomic, visual, motor, and sensory problems being the most common. The specific symptoms are determined by the locations of the lesions within the nervous system, and may include loss of sensitivity or changes in sensation such as tingling, pins and needles or numbness, muscle weakness, blurred vision, very pronounced reflexes, muscle spasms, or difficulty in moving; difficulties with coordination and balance (ataxia); problems with speech or swallowing, visual problems (nystagmus, optic neuritis or double vision), feeling tired, acute or chronic pain, and bladder and bowel difficulties, among others. Difficulties thinking and emotional problems such as depression or unstable mood are also common. Uhthoff's phenomenon, a worsening of symptoms due to exposure to higher than usual temperatures, and Lhermitte's sign, an electrical sensation that runs down the back when bending the neck, are particularly characteristic of MS. The main measure of disability and severity is the expanded disability status scale (EDSS), with other measures such as the multiple sclerosis functional composite being increasingly used in research.

The condition begins in 85% of cases as a clinically isolated syndrome (CIS) over a number of days with 45% having motor or sensory problems, 20% having optic neuritis, and 10% having symptoms related to brainstem dysfunction, while the remaining 25% have more than one of the previous difficulties. The course of symptoms occurs in two main patterns initially: either as episodes of sudden worsening that last a few days to months (called relapses, exacerbations, bouts, attacks, or flare-ups) followed by improvement (85% of cases) or as a gradual worsening over time without periods of recovery (10-15% of cases). A combination of these two patterns may also occur or people may start in a relapsing and remitting course that then becomes progressive later on. Relapses are usually not predictable, occurring without warning. Exacerbations rarely occur more frequently than twice per year. Some relapses, however, are preceded by common triggers and they occur more frequently during spring and summer Similarly, viral infections such as the common cold, influenza, or gastroenteritis increase their risk. Stress may also trigger an attack. Women with MS who become pregnant experience fewer relapses; however, during the first months after delivery the risk increases. Overall, pregnancy does not seem to influence long-term disability. Many events have been found not to affect relapse rates including vaccination, breast feeding, physical trauma, and Uhthoff's phenomenon.

The cause of MS is unknown; however, it is believed to occur as a result of some combination of genetic and environmental factors such as infectious agents. Theories try to combine the data into likely explanations, but none has proved definitive. While there are a number of environmental risk factors and although some are partly modifiable, further research is needed to determine whether their elimination can prevent MS.

The three main characteristics of MS are the formation of lesions in the central nervous system (also called plaques), inflammation, and the destruction of myelin sheaths of neurons. These features interact in a complex and not yet fully understood manner to produce the breakdown of nerve tissue and in turn the signs and symptoms of the disease. Cholesterol crystals are believed to both impair myelin repair and aggravate inflammation. Additionally, MS is believed to be an immune-mediated disorder that develops from an interaction of the individual's genetics and as yet unidentified environmental causes. Damage is believed to be caused, at least in part, by attack on the nervous system by a person's own immune system.

Multiple sclerosis is typically diagnosed based on the presenting signs and symptoms, in combination with supporting medical imaging and laboratory testing. It can be difficult to confirm, especially early on, since the signs and symptoms may be similar to those of other medical problems. The McDonald criteria, which focus on clinical, laboratory, and radiologic evidence of lesions at different times and in different areas, is the most commonly used method of diagnosis with the Schumacher and Poser criteria being of mostly historical significance.

Clinical data alone may be sufficient for a diagnosis of MS if an individual has had separate episodes of neurological symptoms characteristic of the disease. In those who seek medical attention after only one attack, other testing is needed for the diagnosis. The most commonly used diagnostic tools are neuroimaging, analysis of cerebrospinal fluid and evoked potentials. Magnetic resonance imaging of the brain and spine may show areas of demyelination (lesions or plaques). Gadolinium can be administered intravenously as a contrast agent to highlight active plaques and, by elimination, demonstrate the existence of historical lesions not associated with symptoms at the moment of the evaluation. Testing of cerebrospinal fluid obtained from a lumbar puncture can provide evidence of chronic inflammation in the central nervous system. The cerebrospinal fluid is tested for oligoclonal bands of IgG on electrophoresis, which are inflammation markers found in 75-85% of people with MS. The nervous system in MS may respond less actively to stimulation of the optic nerve and sensory nerves due to demyelination of such pathways. These brain responses can be examined using visual- and sensory-evoked potentials.

While the above criteria allow for a non-invasive diagnosis, and even though some state that the only definitive proof is an autopsy or biopsy where lesions typical of MS are detected, currently, as of 2017, there is no single test (including biopsy) that can provide a definitive diagnosis of this disease. Multiple reports new describe the accumulation of tau pathology in brains of MS patients, suggesting that this disorder could be a tauopathy, and that progressive accumulation of tau pathology could underlie the relentless progression of disease.

Several phenotypes (commonly termed types), or patterns of progression, have been described. Phenotypes use the past course of the disease in an attempt to predict the future course. They are important not only for prognosis but also for treatment decisions. Currently, the United States National Multiple Sclerosis Society and the Multiple Sclerosis International Federation, describes four types of MS (revised in 2013):

Clinically isolated syndrome (CIS)

Relapsing-remitting MS (RRMS)

Primary progressive MS (PPMS)

Secondary progressive MS (SPMS)

Relapsing-remitting MS is characterized by unpredictable relapses followed by periods of months to years of relative quiet (remission) with no new signs of disease activity. Deficits that occur during attacks may either resolve or leave problems, the latter in about 40% of attacks and being more common the longer a person has had the disease. This describes the initial course of 80% of individuals with MS. When deficits always resolve between attacks, this is sometimes referred to as benign MS, although people will still build up some degree of disability in the long term. On the other hand, the term malignant multiple sclerosis is used to describe people with MS having reached significant level of disability in a short period. The relapsing-remitting subtype usually begins with a clinically isolated syndrome (CIS). In CIS, a person has an attack suggestive of demyelination, but does not fulfill the criteria for multiple sclerosis. 30 to 70% of persons experiencing CIS later develop MS.

Primary progressive MS occurs in approximately 10-20% of individuals, with no remission after the initial symptoms. It is characterized by progression of disability from onset, with no, or only occasional and minor, remissions and improvements. The usual age of onset for the primary progressive subtype is later than of the relapsing-remitting subtype. It is similar to the age that secondary progressive usually begins in relapsing-remitting MS, around 40 years of age.

Secondary progressive MS occurs in around 65% of those with initial relapsing-remitting MS, who eventually have progressive neurologic decline between acute attacks without any definite periods of remission. Occasional relapses and minor remissions may appear. The most common length of time between disease onset and conversion from relapsing-remitting to secondary progressive MS is 19 years.

Other, unusual types of MS have been described; these include Devic's disease or Neuromyelitis Optica (NMO), Balo concentric sclerosis, Schilder's diffuse sclerosis, and Marburg multiple sclerosis. There is debate on whether they are MS variants or different diseases. Multiple sclerosis behaves differently in children, taking more time to reach the progressive stage. Nevertheless, they still reach it at a lower average age than adults usually do.

Although there is no known cure for multiple sclerosis, several therapies have proven helpful. The primary aims of therapy are returning function after an attack, preventing new attacks, and preventing disability. Starting medications is generally recommended in people after the first attack when more than two lesions are seen on MRI.

As with any medical treatment, medications used in the management of MS have several adverse effects. Alternative treatments are pursued by some people, despite the shortage of supporting evidence.

During symptomatic attacks, administration of high doses of intravenous corticosteroids, such as methylprednisolone, is the usual therapy, with oral corticosteroids seeming to have a similar efficacy and safety profile. Although, in general, effective in the short term for relieving symptoms, corticosteroid treatments do not appear to have a significant impact on long-term recovery. The consequences of severe attacks that do not respond to corticosteroids might be treatable by plasmapheresis.

As of 2017, ten disease-modifying medications are approved by regulatory agencies for relapsing-remitting multiple sclerosis (RRMS). They are interferon β-1a, interferon β-1b, glatiramer acetate, mitoxantrone, natalizumab, fingolimod, teriflunomide, dimethyl fumarate, alemtuzumab, and ocrelizumab. Their cost effectiveness as of 2012 is unclear. In March 2017 the FDA approved ocrelizumab, a humanized anti-CD20 monoclonal antibody, as a treatment for RRMS, with requirements for several Phase IV clinical trials.

In RRMS they are modestly effective at decreasing the number of attacks. The interferons and glatiramer acetate are first-line treatments and are roughly equivalent, reducing relapses by approximately 30%. Early-initiated long-term therapy is safe and improves outcomes. Natalizumab reduces the relapse rate more than first-line agents; however, due to issues of adverse effects is a second-line agent reserved for those who do not respond to other treatments or with severe disease. Mitoxantrone, whose use is limited by severe adverse effects, is a third-line option for those who do not respond to other medications. Treatment of clinically isolated syndrome (CIS) with interferons decreases the chance of progressing to clinical MS. Efficacy of interferons and glatiramer acetate in children has been estimated to be roughly equivalent to that of adults. The role of some newer agents such as fingolimod, teriflunomide, and dimethyl fumarate, as of 2011, is not yet entirely clear.

As of 2017, rituximab was widely used off-label to treat RRMS as well as progressive primary MS. In March 2017 the FDA approved ocrelizumab as a treatment for primary progressive MS, the first drug to gain that approval, with requirements for several Phase IV clinical trials.

As of 2011, only one medication, mitoxantrone, had been approved for secondary progressive MS. In this population tentative evidence supports mitoxantrone moderately slowing the progression of the disease and decreasing rates of relapses over two years.

The disease-modifying treatments have several adverse effects. One of the most common is irritation at the injection site for glatiramer acetate and the interferons (up to 90% with subcutaneous injections and 33% with intramuscular injections). Over time, a visible dent at the injection site, due to the local destruction of fat tissue, known as lipoatrophy, may develop. Interferons may produce flu-like symptoms; some people taking glatiramer experience a post-injection reaction with flushing, chest tightness, heart palpitations, and anxiety, which usually lasts less than thirty minutes. More dangerous but much less common are liver damage from interferons, systolic dysfunction (12%), infertility, and acute myeloid leukemia (0.8%) from mitoxantrone, and progressive multifocal leukoencephalopathy occurring with natalizumab (occurring in 1 in 600 people treated).

Fingolimod may give rise to hypertension and slowed heart rate, macular edema, elevated liver enzymes or a reduction in lymphocyte levels. Tentative evidence supports the short-term safety of teriflunomide, with common side effects including: headaches, fatigue, nausea, hair loss, and limb pain. There have also been reports of liver failure and PML with its use and it is dangerous for fetal development. Most common side effects of dimethyl fumarate are flushing and gastrointestinal problems. While dimethyl fumarate may lead to a reduction in the white blood cell count there were no reported cases of opportunistic infections during trials.

Both medications and neurorehabilitation have been shown to improve some symptoms, though neither changes the course of the disease. Some symptoms have a good response to medication, such as an unstable bladder and spasticity, while others are little changed. For neurologic problems, a multidisciplinary approach is important for improving quality of life; however, it is difficult to specify a ‘core team’ as many health services may be needed at different points in time. Multidisciplinary rehabilitation programs increase activity and participation of people with MS but do not influence impairment level. There is limited evidence for the overall efficacy of individual therapeutic disciplines, though there is good evidence that specific approaches, such as exercise, and psychological therapies are effective. Cognitive behavioral therapy has shown to be moderately effective for reducing MS fatigue.

Over 50% of people with MS may use complementary and alternative medicine, although percentages vary depending on how alternative medicine is defined. The evidence for the effectiveness for such treatments in most cases is weak or absent. Treatments of unproven benefit used by people with MS include dietary supplementation and regimens, vitamin D, relaxation techniques such as yoga, herbal medicine (including medical cannabis), hyperbaric oxygen therapy, [self-infection with hookworms, reflexology, acupuncture, and mindfulness. Regarding the characteristics of users, they are more frequently women, have had MS for a longer time, tend to be more disabled and have lower levels of satisfaction with conventional healthcare.

The expected future course of the disease depends on the subtype of the disease; the individual's sex, age, and initial symptoms; and the degree of disability the person has. Female sex, relapsing-remitting subtype, optic neuritis or sensory symptoms at onset, few attacks in the initial years and especially early age at onset, are associated with a better course.

The average life expectancy is 30 years from the start of the disease, which is 5 to 10 years less than that of unaffected people. Almost 40% of people with MS reach the seventh decade of life. Nevertheless, two-thirds of the deaths are directly related to the consequences of the disease. Suicide is more common, while infections and other complications are especially dangerous for the more disabled. Although most people lose the ability to walk before death, 90% are capable of independent walking at 10 years from onset, and 75% at 15 years.

C. Frontotemporal Dementia

Frontotemporal dementia (FTD), formerly known as disinhibition-dementia-parkinsonism-amyotrophy complex (DDPAC), is a neurodegenerative disease characterized by severe frontotemporal lobar degeneration. The disorder was first identified in 1994 by Kirk Wilhelmsen and colleagues, who distinguished it from Alzheimer's disease and Lewy body dementia based on the fact that it did not manifest with amyloid plaques, neurofibrillary tangles, or Lewy bodies. Second only to Alzheimer's disease (AD) in prevalence, FTD accounts for 20% of pre-senile dementia cases. Symptoms can begin to appear on average around 45 to 65 years of age, regardless of gender. The most common symptoms include significant changes in social and personal behavior, as well as a general blunting of emotions. Symptoms progress at a rapid, steady rate. Patients suffering from the disease can survive between 2-10 years. Eventually patients will need 24-hour care for daily function. Because FTD often occurs in younger people (i.e., in their 40's or 50's), it can severely affect families Patients often still have children living in the home Financially, it can be devastating as the disease strikes at the time of life that is often the top wage-earning years. Currently, there is no cure for FTD. Treatments are available to manage the behavioral symptoms. Disinhibition and compulsive behaviors can be controlled by selective serotonin reuptake inhibitors (SSRIs). Although Alzheimer's and FTD share certain symptoms, they cannot be treated with the same pharmacological agents because the cholinergic systems are not affected in FTD.

FTD is traditionally difficult to diagnose due to the heterogeneity of the associated symptoms. Symptoms are classified into three groups based on the functions of the frontal and temporal lobes. Behavioral variant FTD (bvFTD) exhibits symptoms of lethargy and aspontaneity on the one hand, and disinhibition on the other. Apathetic patients may become socially withdrawn and stay in bed all day or no longer take care of themselves. Disinhibited patients can make inappropriate (sometimes sexual) comments or perform inappropriate acts. Patients with FTD can sometimes get into trouble with the law because of inappropriate behavior such as stealing or speeding. Recent findings indicate that psychotic symptoms are rare in FTD, possibly due to limited temporal-limbic involvement. Among FTD patients, approximately 2% have delusions, sometimes with paranoid ideation. Hallucinations are rare. These psychotic symptoms are significantly less prevalent than what is seen in AD patients, where approximately 20% have delusions and paranoia. Progressive nonfluent aphasia (PNFA) presents with a breakdown in speech fluency due to articulation difficulty, phonological and/or syntactic errors but preservation of word comprehension. Semantic dementia (SD) can be found in some patients that remain fluent with normal phonology and syntax but increasing difficulty with naming and word comprehension. It has been researched that some may even go through depression and lose their inhibitions and exhibit antisocial behavior.

FTD patients tend to struggle with binge eating and compulsive behaviors. These binge eating habits are often associated with abnormal eating behavior including overeating, stuffing oneself with food, changes in food preferences (cravings for more sweets, carbohydrates), eating inedible objects and snatching food from others. Recent findings have indicated that the neural structures responsible for eating changes in FTD include atrophy in the right ventral insula, striatum and orbitofrontal cortex on structural MRI voxel-based morphometry (right hemisphere).

Executive function is the cognitive skill of planning and organizing. Most FTD patients become unable to perform skills that require complex planning or sequencing. In addition to the characteristic cognitive dysfunction, a number of primitive reflexes known as frontal release signs are often able to be elicited. Usually, the first of these frontal release signs to appear is the palmomental reflex which appears relatively early in the disease course whereas the palmar grasp reflex and rooting reflex appear late in the disease course. The following abilities in the FTD patients are preserved: perception, spatial skills, memory, praxis, The following abilities in FTD patients are affected: social behavior/conduct, regulation of emotion, ability to focus, utilization behavior (neurobehavioral disorder where the patients grab objects in view and start to conduct the right behavior at the wrong time), and inappropriate speech/actions.

In rare cases, FTD can occur in patients with motor neuron disease (MND) (typically amyotrophic lateral sclerosis). The prognosis for people with MND is worse when combined with FTD, shortening survival by about a year. A number of case series have now been published looking at the pathological basis of frontotemporal dementia. As with other syndromes associated with frontotemporal lobar degeneration (FTLD), a number of different pathologies are associated with FTD:
Tau pathology: In a healthy individual, tau proteins stabilize microtubules, which are a major component of the cytoskeleton. Examples include Pick's disease, now also referred to as FTLD-tau, and other tau-positive pathology including FTDP-17, corticobasal degeneration, and progressive supranuclear palsy. Approximately 50% of FTD cases will present with tau pathology at post-mortem.
TDP-43 pathology: This disease form was previously described as dementia with ubiquitin positive, tau- and alpha-synuclein negative inclusions with and without motor neuron degeneration. FTLD-TDP43 accounts for approximately 40% of FTD(±MND).
FUS pathology: Cases with underlying FUS pathology tend to present with behavioral variant FTD (bvFTD), but the correlation is by no means reliable enough to predict the post-mortem pathology. FTLD-FUS represents only 5-10% of clinically diagnosed FTD.

Dementia lacking distinctive histology (DLDH) is a rare entity and represents the remaining small percentage of FTD that cannot be positively diagnosed as any of the above at post-mortem.

In rare cases, patients with clinical FTD were found to have changes consistent with Alzheimer's disease on autopsy Evidence suggests that FTD selectively impairs spindle neurons, a type of neuron which has only been found in the brains of humans, great apes, and whales. Deficiencies of the micronutrients folate and B12 have been associated with cognitive impairment in individuals with FTD. Chronic folate deficiency has also been implicated in cerebral atrophy, leading to neurological impairment.

Structural MRI scans often reveal frontal lobe and/or anterior temporal lobe atrophy but in early cases the scan may seem normal. Atrophy can be either bilateral or asymmetric. Registration of images at different time points (e.g., one year apart) can show evidence of atrophy that otherwise (at individual time points) may be reported as normal. Many research groups have begun using techniques such as magnetic resonance spectroscopy, functional imaging and cortical thickness measurements in an attempt to offer an earlier diagnosis to the FTD patient. Fluorine-18-Fluorodeoxyglucose Positron Emission Tomography (FDG-PET) scans classically show frontal and/or anterior temporal hypometabolism, which helps differentiate the disease from Alzheimer's disease. The PET scan in Alzheimer's disease classically shows biparietal hypometabolism. Meta-analyses based on imaging methods have shown that frontotemporal dementia mainly affects a frontomedial network discussed in the context of social cognition or ‘theory of mind’. This is entirely in keeping with the notion that on the basis of cognitive neuropsychological evidence, the ventromedial prefrontal cortex is a major locus of dysfunction early on in the course of the behavioural variant of frontotemporal degeneration. The language subtypes of frontotemporal lobar degeneration (semantic dementia and progressive non-fluent aphasia) can be regionally dissociated by imaging approaches in vivo.

The confusion between Alzheimer's and FTD is justifiable due to the similarities between their initial symptoms. Patients do not have difficulty with movement and other motor tasks. As FTD symptoms appear, it is difficult to differentiate between a diagnosis of Alzheimer's disease and FTD. There are distinct differences in the behavioral and emotional symptoms of the two dementias, notably, the blunting of emotions seen in FTD patients. In the early stages of FTD, anxiety and depression are common, which may result in an ambiguous diagnosis. However, over time, these ambiguities fade away as this dementia progresses and defining symptoms of apathy, unique to FTD, start to appear.

In vivo brain imaging of tau aggregation in frontotemporal dementia using [¹⁸F]FDDNP positron emission tomography is more visual and has enhanced the ability to have a deeper understanding in frontal temporal dementia. Previous fluorescent microscopy studies of Alzheimer's disease (AD) brain specimens have shown that [¹⁸F]FDDNP displays an excellent visualization of interneuronal neurofibrillary tangles (NFTs). Visual images of [¹⁸F]FDDNP-PET images emphasized a frontal signal in FTD compared to prominent temporal signals in AD. [¹⁸F]FDDNP-PET has allowed the enhanced visualization of tauopathies in patients. This has aided in differentiating FTD from parietal and temporal signals in AD. Further, the ability of [¹⁸F]FDDNP to entitle tauopathies in vivo gives a tool for monitoring the effect of therapies to eliminate NFT accumulation. Recent studies over several years have developed new criteria for the diagnosis of behavioral variant frontotemporal dementia (bvFTD). Six distinct clinical features have been identified as symptoms of bvFTD:

Disinhibition

Apathy/Inertia

Loss of Sympathy/Empathy

Perseverative/compulsive behaviors

Hyperorality

Dysexecutive neuropsychological profile

Of the six features, three must be present in a patient to diagnose one with possible bvFTD. Similar to standard FTD, the primary diagnosis stems from clinical trials that identify the associated symptoms, instead of imaging studies. The above criteria are used to distinguish bvFTD from disorders such as Alzheimer's and other causes of dementia. In addition, the new criteria allow for a diagnostic hierarchy distinguished possible, probable, and definite bvFTD based on the number of symptoms present.

A higher proportion of FTD cases seem to have a familial component than more common neurodegenerative diseases like Alzheimer's disease. More and more mutations and genetic variants are being identified all the time, so the lists of genetic influences require consistent updating. Tau-positive frontotemporal dementia with parkinsonism (FTDP-17) is caused by mutations in the MAPT gene on chromosome 17 that encodes the Tau protein It has been determined that there is a direct relationship between the type of tau mutation and the neuropathology of gene mutations. The mutations at the splice junction of exon 10 of tau lead to the selective deposition of the repetitive tau in neurons and glia. The pathological phenotype associated with mutations elsewhere in tau is less predictable with both typical neurofibrillary tangles (consisting of both 3 repeat and 4 repeat tau) and Pick bodies (consisting of only 3 repeat tau) having been described. The presence of tau deposits within glia is also variable in families with mutations outside of exon 10. This disease is now informally designated FTDP-17T. FTD shows a linkage to the region of the tau locus on chromosome 17, but it is believed that there are two loci leading to FTD within megabases of each other on chromosome 17. FTD caused by FTLD-TDP43 has numerous genetic causes. Some cases are due to mutations in the GRN gene, also located on chromosome 17. Others are caused by VCP mutations, although these patients present with a complex mixture of Inclusion body myopathy, Paget's disease of bone, and FTD. The most recent addition to the list is a hexanucleotide repeat expansion in the promotor region of C9ORF72. Only one or two cases have been reported describing TARDBP (the TDP-43 gene) mutations in a clinically pure FTD (FTD without MND).

D. Progressive Supranuclear Palsy

Progressive supranuclear palsy (PSP; or the Steele-Richardson-Olszewski syndrome, after the doctors who described it in 1963) is a degenerative disease involving the gradual deterioration and death of specific volumes of the brain. Males and females are affected approximately equally and there is no racial, geographical or occupational predilection. Approximately six people per 100,000 population have PSP. It has been described as a tauopathy. There is currently no effective treatment or cure for PSP, although some of the symptoms can respond to nonspecific measures. The average age at symptoms onset is 63 and survival from onset averages 7 years with a wide variance. Pneumonia is a frequent cause of death.

The initial symptoms in two-thirds of cases are loss of balance, lunging forward when mobilizing, fast walking, bumping into objects or people, and falls. Other common early symptoms are changes in personality, general slowing of movement, and visual symptoms. Later symptoms and signs are dementia (typically including loss of inhibition and ability to organize information), slurring of speech, difficulty swallowing, and difficulty moving the eyes, particularly in the vertical direction. The latter accounts for some of the falls experienced by these patients as they are unable to look up or down. Some of the other signs are poor eyelid function, contracture of the facial muscles, a backward tilt of the head with stiffening of the neck muscles, sleep disruption, urinary incontinence and constipation.

The visual symptoms are of particular importance in the diagnosis of this disorder. Patients typically complain of difficulty reading due to the inability to look down well. Notably, the ophthalmoparesis experienced by these patients mainly concerns voluntary eye movement and the inability to make vertical saccades, which is often worse with downward saccades. Patients tend to have difficulty looking down (a downgaze palsy) followed by the addition of an upgaze palsy. This vertical gaze paresis will correct when the examiner passively rolls the patient's head up and down as part of a test for the oculocephalic reflex. Involuntary eye movement, as elicited by Bell's phenomenon, for instance, may be closer to normal. On close inspection, eye movements called “square-wave jerks” may be visible when the patient fixes at distance. These are fine movements that can be mistaken for nystagmus, except that they are saccadic in nature with no smooth phase. Although healthy individuals also make square-wave jerk movements, PSP patients make slower square-wave jerk movements, with smaller vertical components. Assessment of these square-wave jerks and diminished vertical saccades is especially useful for diagnosing progressive supranuclear palsy, because these movements set PSP patients apart from other parkinsonian patients. Difficulties with convergence (convergence insufficiency), where the eyes come closer together while focusing on something near, like the pages of a book, is typical. Because the eyes have trouble coming together to focus at short distances, the patient may complain of diplopia (double vision) when reading.

Cardinal manifestations include supranuclear ophthalmoplegia, neck dystonia, Parkinsonism, Pseudobulbar palsy, behavioral and cognitive impairment, imbalance and walking difficulty, and frequent falls.

The cause of PSP is unknown. Fewer than 1% of those with PSP have a family member with the same disorder. A variant in the gene for tau protein called the H1 haplotype, located on chromosome 17, has been linked to PSP. Nearly all people with PSP received a copy of that variant from each parent, but this is true of about two-thirds of the general population. Therefore, the H1 haplotype appears to be necessary but not sufficient to cause PSP. Other genes, as well as environmental toxins, are being investigated as possible contributors to the cause of PSP.

The affected brain cells are both neurons and glial cells. The neurons display neurofibrillary tangles, which are clumps of tau protein, a normal part of a brain cell's internal structural skeleton. These tangles are often different from those seen in Alzheimer's disease, but may be structurally similar when they occur in the cerebral cortex. Their chemical composition is usually different, however, and is similar to that of tangles seen in corticobasal degeneration. Tufts of tau protein in astrocytes, or tufted astrocytes, are also considered diagnostic. Unlike globose NFTs, they may be more widespread in the cortex. Lewy bodies are seen in some cases, but it is not clear whether this is a variant or an independent co-existing process, and in some cases PSP can coexist with corticobasal degeneration, Parkinson's and/or Alzheimer's Disease, particularly in older patients.

The principal areas of the brain affected are the:
basal ganglia, particularly the subthalamic nucleus, substantia nigra and globus pallidus;
brainstem, particularly the portion of the midbrain where “supranuclear” eye movement resides;
cerebral cortex, particularly that of the frontal lobes;
dentate nucleus of the cerebellum;
spinal cord, particularly the area where some control of the bladder and bowel resides

Some consider PSP, corticobasal degeneration, and frontotemporal dementia to be variations of the same disease. Others consider them separate diseases. PSP has been shown occasionally to co-exist with Pick's disease.

MRI is often done to diagnose PSP. MRI may show atrophy in the midbrain with preservation of the pons giving a “hummingbird” sign appearance and Mickey Mouse sign. PSP is frequently misdiagnosed as Parkinson's disease because of the slowed movements and gait difficulty, or as Alzheimer's disease because of the behavioral changes. It is one of a number of diseases collectively referred to as Parkinson plus syndromes. A poor response to levodopa along with symmetrical onset can help differentiate this disease from PD. Also, patients with the Richardson variant tend to have an upright or arched-back posture as opposed to the stooped-forward posture of other Parkinsonian disorders, although PSP-Parkinsonism (see below) may show the stooped posture. Early falls are characteristic, especially with Richardson-syndrome.

There is no known cure for PSP and management is primarily supportive. PSP cases are often split into two subgroups, PSP-Richardson, the classic type, and PSP-Parkinsonism, where a short-term response to levodopa can be obtained. Dyskinesia is an occasional but rare complication of treatment. Amantadine is also sometimes helpful. After a few years the Parkinsonian variant tends to take on Richardson features. Other variants have been described. Botox can be used to treat neck dystonia and blephrospasm, but this can aggravate dysphagia.

Studies have suggested that rivastigmine may help with cognitive aspects, but the authors of both studies have suggested a larger sampling be used. There is some evidence that the hypnotic zolpidem may improve motor function and eye movements, but only from small-scale studies.

Patients with PSP usually seek or are referred to occupational therapy, speech-language pathology for motor speech changes typically a spastic-ataxic dysarthria, and physical therapy for balance and gait problems with reports of frequent falls. Evidence-based approaches to rehabilitation in PSP are lacking, and currently the majority of research on the subject consists of case reports involving only a small number of patients.

Case reports of rehabilitation programs for patients with PSP generally include limb-coordination activities, tilt-board balancing, gait training, strength training with progressive resistive exercises and isokinetic exercises and stretching of the neck muscles. While some case reports suggest that physiotherapy can offer improvements in balance and gait of patients with PSP, the results cannot be generalized across all patients with PSP as each case report only followed one or two patients. The observations made from these case studies can be useful, however, in helping to guide future research concerning the effectiveness of balance and gait training programs in the management of PSP.

Individuals with PSP are often referred to occupational therapists to help manage their condition and to help enhance their independence. This may include being taught to use mobility aids. Due to their tendency to fall backwards, the use of a walker, particularly one that can be weighted in the front, is recommended over a cane. The use of an appropriate mobility aid will help to decrease the individual's risk of falls and make them safer to ambulate independently in the community. Due to their balance problems and irregular movements individuals will need to spend time learning how to safely transfer in their homes as well as in the community. This may include rising from and sitting in chairs safely.

Due to the progressive nature of this disease, all individuals eventually lose their ability to walk and will need to progress to using a wheelchair. Severe dysphagia often follows, and at this point death is often a matter of months.

E. Corticobasal Degeneration

Corticobasal ganglionic degeneration (CBgD) or corticobasal ganglionic degeneration (CBGD) is a rare, progressive neurodegenerative disease involving the cerebral cortex and the basal ganglia. CBgD symptoms typically begin in people from 50 to 70 years of age, and the average disease duration is six years. It is characterized by marked disorders in movement and cognitive dysfunction, and is classified as one of the Parkinson plus syndromes. Clinical diagnosis is difficult, as symptoms of CBgD are often similar to those of other disorders, such as Parkinson's disease (PD), progressive supranuclear palsy (PSP), and dementia with Lewy bodies (DLB). Due to the various clinical presentations associated with CBgD, a final diagnosis can only be made upon neuropathologic examination.

Because CBgD is progressive, a standard set of diagnostic criteria can be used, which is centered on the disease's evolution. Included in these fundamental features are problems with cortical processing, dysfunction of the basal ganglia, and a sudden and detrimental onset. Psychiatric and cognitive dysfunctions, although present in CBgD, are much less prevalent and lack establishment as common indicators of the presence of the disease.

Some of the most prevalent symptom types in people exhibiting CBgD pertain to identifiable movement disorders and problems with cortical processing. These symptoms are initial indicators of the presence of the disease. Each of the associated movement complications typically appear asymmetrically and the symptoms are not observed uniformly throughout the body. For example, a person exhibiting an alien hand syndrome (explained later) in one hand, will not correspondingly display the same symptom in the contralateral limb. Predominant movement disorders and cortical dysfunctions associated with CBgD include Parkinsonism, Alien hand syndrome, Apraxia (ideomotor apraxia and limb-kinetic apraxia) and Aphasia.

The presence of Parkinsonism as a clinical symptom of CBgD is largely responsible for complications in developing unique diagnostic criteria for the disease. Other such diseases in which Parkinsonism forms an integral diagnostic characteristic are PD and PSP. Parkinsonism in CBgD is largely present in an extremity such as the arm, and is always asymmetric. It has been suggested that non-dominant arm is involved more often. Common associated movement dysfunctions that comprise Parkinsonism are rigidity, bradykinesia, and gait disorder, with limb rigidity forming the most typical manifestation of parkinsonism in CBgD. Despite being relatively indistinct, this rigidity can lead to disturbances in gait and correlated movements. Bradykinesia in CBgD occurs when there is notable slowing in the completion of certain movements in the limbs. In an associated study, it was determined that, three years following first diagnosis, 71% of persons with CBgD demonstrate the presence of bradykinesia.

Alien hand syndrome has been shown to be prevalent in roughly 60% of those people diagnosed with CBgD. This disorder involves the failure of an individual to control the movements of his or her hand, which results from the sensation that the limb is “foreign.” The movements of the alien limb are a reaction to external stimuli and do not occur sporadically or without stimulation. The presence of an alien limb has a distinct appearance in CBgD, in which the diagnosed individual may have a “tactile mitgehen.” This mitgehen (German, meaning “to go with”) is relatively specific to CBgD, and involves the active following of an experimenter's hand by the subject's hand when both hands are in direct contact. Another, rarer form of alien hand syndrome has been noted in CBgD, in which an individual's hand displays an avoidance response to external stimuli. Additionally, sensory impairment, revealed through limb numbness or the sensation of prickling, may also concurrently arise with alien hand syndrome, as both symptoms are indicative of cortical dysfunction. Like most of the movement disorders, alien hand syndrome also presents asymmetrically in those diagnosed with CBgD.

Ideomotor apraxia (IMA), although clearly present in CBgD, often manifests atypically due to the additional presence of bradykinesia and rigidity in those individuals exhibiting the disorders. The IMA symptom in CBgD is characterized by the inability to repeat or mimic particular movements (whether significant or random) both with or without the implementation of objects. This form of IMA is present in the hands and arms, while IMA in the lower extremities may cause problems with walking. Those with CBgD that exhibit IMA may appear to have trouble initiating walking, as the foot may appear to be fixed to floor. This can cause stumbling and difficulties in maintaining balance. IMA is associated with deterioration in the premotor cortex, parietal association areas, connecting white matter tracts, thalamus, and basal ganglia. Some individuals with CBgD exhibit limb-kinetic apraxia, which involves dysfunction of more fine motor movements often performed by the hands and fingers.

Aphasia in CBgD is revealed through the inability to speak or a difficulty in initiating spoken dialogue and falls under the non-fluent (as opposed to fluent or flowing) subtype of the disorder. This may be related to speech impairment such as dysarthria, and thus is not a true aphasia, as aphasia is related to a change in language function, such as difficulty retrieving words or putting words together to form meaningful sentences. The speech and/or language impairments in CBgD result in disconnected speech patterns and the omission of words. Individuals with this symptom of CBgD often lose the ability to speak as the disease progresses.

Psychiatric problems associated with CBgD often present as a result of the debilitating symptoms of the disease. Prominent psychiatric and cognitive conditions cited in individuals with CBgD include dementia, depression, and irritability, with dementia forming a key feature that sometimes leads to the misdiagnosis of CBgD as another cognitive disorder such as Alzheimer's disease (AD). Frontotemporal dementia can be an early feature.

Neuropathological findings associated with CBgD include the presence of astrocytic abnormalities within the brain and improper accumulation of the protein tau (referred to as tauopathy). Postmortem histological examination of the brains of individuals diagnosed with CBgD reveal unique characteristics involving the astrocytes in localized regions. The typical procedure used in the identification of these astroglial inclusions is the Gallyas-Braak staining method. This process involves exposing tissue samples to a silver staining material which marks for abnormalities in the tau protein and astroglial inclusions. Astroglial inclusions in CBgD are identified as astrocytic plaques, which present as annularly displays of blurry outgrowths from the astrocyte. A recent study indicated that CBgD produces a high density of astrocytic plaques in the anterior portion of the frontal lobe and in the premotor area of the cerebral cortex.

The protein tau is an important microtubule-associated protein (MAP), and is typically found in neuronal axons. However, malfunctioning of the development of the protein can result in unnatural, high-level expression in astrocytes and glial cells. As a consequence, this is often responsible for the astrocytic plaques prominently noted in histological CBgD examinations. Although they are understood to play a significant role in neurodegenerative diseases such as CBgD, their precise effect remains a mystery.

One of the most significant problems associated with CBgD is the inability to perform a definitive diagnosis while an individual exhibiting the symptoms associated with CBgD is still alive. A clinical diagnosis of CBgD is performed based upon the specified diagnostic criteria, which focus mainly on the symptoms correlated with the disease. However, this often results in complications as these symptoms often overlap with numerous other neurodegenerative diseases. Frequently, a differential diagnosis for CBgD is performed, in which other diseases are eliminated based on specific symptoms that do not overlap. However, some of the symptoms of CBgD used in this process are rare to the disease, and thus the differential diagnosis cannot always be used.

Postmortem diagnosis provides the only true indication of the presence of CBgD. Most of these diagnoses utilize the Gallyas-Braak staining method, which is effective in identifying the presence of astroglial inclusions and coincidental tauopathy.

Progressive supranuclear palsy (PSP) is frequently the disease most often confused with CBgD. Both PSP and CBgD result in similar symptoms, and both display tauopathies upon histological inspection. However, it has been noted that tauopathy in PSP results in tuft-shaped astrocytes in contrast with the doughnut-shaped astrocytic plaques found as a result of CBgD.

Individuals diagnosed with PD often exhibit similar movement dysfunction as those diagnosed with CBgD, which adds complexity to its diagnosis. Some other neurodegenerative diseases including Alzheimer's disease (AD), dementia with Lewy bodies (DLB), and frontotemporal dementia (FTD) also show commonalities with CBgD.

F. Chronic Traumatic Encephalopathy

Chronic traumatic encephalopathy (CTE), formerly known as dementia pugilistica, is a neurodegenerative disease found in people who have had multiple head injuries. Symptoms may include behavioral problems, mood problems, and problems with thinking This typically does not begin until years after the injuries. It often gets worse over time and can result in dementia. It is unclear if the risk of suicide is altered.

Most documented cases have occurred in athletes involved in contact sports such as boxing, American football, wrestling, ice hockey, rugby and soccer. Other risk factors include being in the military, prior domestic violence, and repeated banging of the head. The exact amount of trauma required for the condition to occur is unknown. Definitive diagnosis can only occur at autopsy. It is a form of tauopathy.

As of 2018, there is no specific treatment. Rates of disease have been found to be about 30% among those with a history of multiple head injuries. Population rates, however, are unclear. Research into brain damage as a result of repeated head injuries began in the 1920s, at which time the condition was known as dementia pugilistica or “punch drunk syndrome.” Changing the rules in some sports has been discussed as a means of prevention.

Symptoms of CTE, which occur in four stages, generally appear 8 to 10 years after an athlete experiences repetitive mild traumatic brain injury.

First-stage symptoms include attention deficit hyperactivity disorder as well as confusion, disorientation, dizziness, and headaches. Second-stage symptoms include memory loss, social instability, impulsive behavior, and poor judgment. Third and fourth stages include progressive dementia, movement disorders, hypomimia, speech impediments, sensory processing disorder, tremors, vertigo, deafness, depression and suicidality.

Additional symptoms include dysarthria, dysphagia, cognitive disorder such as amnesia, and ocular abnormalities, such as ptosis.

The condition manifests as dementia, or declining mental ability, problems with memory, dizzy spells or lack of balance to the point of not being able to walk under one's own power for a short time and/or Parkinsonism, or tremors and lack of coordination. It can also cause speech problems and an unsteady gait. Patients with CTE may be prone to inappropriate or explosive behavior and may display pathological jealousy or paranoia.

The neuropathological appearance of CTE is distinguished from other tauopathies, such as Alzheimer's disease. The four clinical stages of observable CTE disability have been correlated with tau pathology in brain tissue, ranging in severity from focal perivascular epicenters of neurofibrillary tangles in the frontal neocortex to severe tauopathy affecting widespread brain regions.

The primary physical manifestations of CTE include a reduction in brain weight, associated with atrophy of the frontal and temporal cortices and medial temporal lobe. The lateral ventricles and the third ventricle are often enlarged, with rare instances of dilation of the fourth ventricle. Other physical manifestations of CTE include anterior cavum septi pellucidi and posterior fenestrations, pallor of the substantia nigra and locus ceruleus, and atrophy of the olfactory bulbs, thalamus, mammillary bodies, brainstem and cerebellum. As CTE progresses, there may be marked atrophy of the hippocampus, entorhinal cortex, and amygdala.

On a microscopic scale, the pathology includes neuronal loss, tau deposition, TAR DNA-binding Protein 43 (TDP 43) deposition, white matter changes, and other abnormalities. The tau deposition occurs as dense neurofibrillary tangles (NFT), neurites, and glial tangles, which are made up of astrocytes and other glial cells Beta-amyloid deposition is a relatively uncommon feature of CTE.

A small group of individuals with CTE have chronic traumatic encephalomyopathy (CTEM), which is characterized by symptoms of motor-neuron disease and which mimics amyotrophic lateral sclerosis (ALS). Progressive muscle weakness and balance and gait problems (problems with walking) seem to be early signs of CTEM.

Exosome vesicles created by the brain are potential biomarkers of TBI, including CTE. A subtype of CTE is dementia pugilistica or boxer's dementia (from Latin pugilator—boxer) as it was initially found in those with a history of boxing, also called “punch-drunk syndrome”.

Loss of neurons, scarring of brain tissue, collection of proteinaceous, senile plaques, hydrocephalus, attenuation of the corpus callosum, diffuse axonal injury, neurofibrillary tangles, and damage to the cerebellum are implicated in the syndrome. The condition may be etiologically related to Alzheimer's disease. Neurofibrillary tangles have been found in the brains of dementia pugilistica patients, but not in the same distribution as is usually found in people with Alzheimer's. One group examined slices of brain from patients having had multiple mild traumatic brain injuries and found changes in the cells' cytoskeletons, which they suggested might be due to damage to cerebral blood vessels.

Increased exposure to concussions and sub-concussive blows is regarded as the most important risk factor, which can depend on the total number of fights, number of knockout losses, the duration of career, fight frequency, age of retirement, and boxing style.

Currently, CTE can only be definitively diagnosed by direct tissue examination after death, including full and immunohistochemical brain analyses.

The lack of in vivo techniques to show distinct biomarkers for CTE is the reason CTE cannot currently be diagnosed while a person is alive. The only known diagnosis for CTE occurs by studying the brain tissue after death. Concussions are non-structural injuries and do not result in brain bleeding, which is why most concussions cannot be seen on routine neuroimaging tests such as CT or MRI. Acute concussion symptoms (those that occur shortly after an injury) should not be confused with CTE. Differentiating between prolonged post-concussion syndrome (PCS, where symptoms begin shortly after a concussion and last for weeks, months, and sometimes even years) and CTE symptoms can be difficult. Research studies are currently examining whether neuroimaging can detect subtle changes in axonal integrity and structural lesions that can occur in CTE. Recently, more progress in in vivo diagnostic techniques for CTE has been made, using DTI, fMRI, MRI, and MRS imaging; however, more research needs to be done before any such techniques can be validated.

PET tracers that bind specifically to tau protein are desired to aid diagnosis of CTE in living individuals. One candidate is the tracer [¹⁸F]FDDNP, which is retained in the brain in individuals with a number of dementing disorders such as Alzheimer's disease, Down syndrome, progressive supranuclear palsy, familial frontotemporal dementia, and Creutzfeldt-Jakob disease. In a small study of 5 retired NFL players with cognitive and mood symptoms, the PET scans revealed accumulation of the tracer in their brains. However, [¹⁸F]FDDNP binds to beta-amyloid and other proteins as well. Moreover, the sites in the brain where the tracer was retained were not consistent with the known neuropathology of CTE. A more promising candidate is the tracer [¹⁸F]-T807, which binds only to tau. It is being tested in several clinical trials.

A putative biomarker for CTE is the presence in serum of autoantibodies against the brain. The autoantibodies were detected in football players who experienced a large number of head hits but no concussions, suggesting that even sub-concussive episodes may be damaging to the brain. The autoantibodies may enter the brain by means of a disrupted blood-brain barrier, and attack neuronal cells which are normally protected from an immune onslaught. Given the large numbers of neurons present in the brain (86 billion), and considering the poor penetration of antibodies across a normal blood-brain barrier, there is an extended period of time between the initial events (head hits) and the development of any signs or symptoms. Nevertheless, autoimmune changes in blood of players may consist the earliest measurable event predicting CTE.

### II. MONOCLONAL ANTIBODIES AND PRODUCTION THEREOF

An “isolated antibody” is one that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular embodiments, the antibody is purified: (1) to greater than 95% by weight of antibody as determined by the Lowry method, and most particularly more than 99% by weight; (2) to a degree sufficient to obtain at least 15 residues of N-terminal or internal amino acid sequence by use of a spinning cup sequenator; or (3) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, isolated antibody will be prepared by at least one purification step.

The basic four-chain antibody unit is a heterotetrameric glycoprotein composed of two identical light (L) chains and two identical heavy (H) chains. An IgM antibody consists of 5 basic heterotetramer units along with an additional polypeptide called J chain, and therefore contain 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four C_{H} domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H1}). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

The L chain from any vertebrate species can be assigned to one of two clearly distinct types, called kappa and lambda based on the amino acid sequences of their constant domains (C_{L}). Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated alpha, delta, epsilon, gamma and mu, respectively. They gamma and alpha classes are further divided into subclasses on the basis of relatively minor differences in C_{H} sequence and function, humans express the following subclasses: IgG1, IgG2, IgG3, IgG4, IgA1, and IgA2.

The term “variable” refers to the fact that certain segments of the V domains differ extensively in sequence among antibodies. The V domain mediates antigen binding and defines specificity of a particular antibody for its particular antigen. However, the variability is not evenly distributed across the 110-amino acid span of the variable regions. Instead, the V regions consist of relatively invariant stretches called framework regions (FRs) of 15-30 amino acids separated by shorter regions of extreme variability called “hypervariable regions” that are each 9-12 amino acids long. The variable regions of native heavy and light chains each comprise four FRs, largely adopting a beta-sheet configuration, connected by three hypervariable regions, which form loops connecting, and in some cases forming part of, the beta-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{sub}H when numbered in accordance with AHo; Honneger, A. and Plunkthun, A. J. Mol. Biol. 309:657-670 (2001)).

By “germline nucleic acid residue” is meant the nucleic acid residue that naturally occurs in a germline gene encoding a constant or variable region. “Germline gene” is the DNA found in a germ cell (i.e., a cell destined to become an egg or in the sperm). A “germline mutation” refers to a heritable change in a particular DNA that has occurred in a germ cell or the zygote at the single-cell stage, and when transmitted to offspring, such a mutation is incorporated in every cell of the body. A germline mutation is in contrast to a somatic mutation which is acquired in a single body cell. In some cases, nucleotides in a germline DNA sequence encoding for a variable region are mutated (i.e., a somatic mutation) and replaced with a different nucleotide.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” is not to be construed as requiring production of the antibody by any particular method. For example, the monoclonal antibodies useful in the present disclosure may be prepared by the hybridoma methodology first described by Kohler et al., Nature, 256:495 (1975), or may be made using recombinant DNA methods in bacterial, eukaryotic animal or plant cells (see, e.g., U.S. Pat. No. 4,816,567) after single cell sorting of an antigen specific B cell, an antigen specific plasmablast responding to an infection or immunization, or capture of linked heavy and light chains from single cells in a bulk sorted antigen specific collection. The “monoclonal antibodies” may also be isolated from phage antibody libraries using the techniques described in Clackson et al., Nature, 352:624-628 (1991) and Marks et al., J. Mol. Biol., 222:581-597 (1991), for example.

A. General Methods

It will be understood that monoclonal antibodies binding to tau will have several applications. These include the production of diagnostic kits for use in detecting and diagnosing tauopathies, as well as for treating the same. In these contexts, one may link such antibodies to diagnostic or therapeutic agents, use them as capture agents or competitors in competitive assays, or use them individually without additional agents being attached thereto. The antibodies may be mutated or modified, as discussed further below. Methods for preparing and characterizing antibodies are well known in the art.

The methods for generating monoclonal antibodies (MAbs) generally begin along the same lines as those for preparing polyclonal antibodies. The first step for both these methods is immunization of an appropriate host or identification of subjects who are immune due to prior natural infection or vaccination with a licensed or experimental vaccine. As is well known in the art, a given composition for immunization may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimyde and bis-biazotized benzidine. As also is well known in the art, the immunogenicity of a particular immunogen composition can be enhanced by the use of non-specific stimulators of the immune response, known as adjuvants. Exemplary and preferred adjuvants in animals include complete Freund's adjuvant (a non-specific stimulator of the immune response containing killed Mycobacterium tuberculosis), incomplete Freund's adjuvants and aluminum hydroxide adjuvant and in humans include alum, CpG, MFP59 and combinations of immunostimulatory molecules (“Adjuvant Systems”, such as AS01 or AS03). Additional experimental forms of inoculation to induce tau-specific B cells is possible, including nanoparticle vaccines, or gene-encoded antigens delivered as DNA or RNA genes in a physical delivery system (such as lipid nanoparticle or on a gold biolistic bead), and delivered with needle, gene gun, transcutaneous electroporation device. The antigen gene also can be carried as encoded by a replication competent or defective viral vector such as adenovirus, adeno-associated virus, poxvirus, herpesvirus, or alphavirus replicon, or alternatively a virus like particle.

In the case of human antibodies against natural pathogens, a suitable approach is to identify subjects that have been exposed to the pathogens, such as those who have been diagnosed as having contracted the disease, or those who have been vaccinated to generate protective immunity against the pathogen or to test the safety or efficacy of an experimental vaccine. Circulating anti-pathogen antibodies can be detected, and antibody encoding or producing B cells from the antibody-positive subject may then be obtained.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster injection, also may be given. The process of boosting and titering is repeated until a suitable titer is achieved. When a desired level of immunogenicity is obtained, the immunized animal can be bled and the serum isolated and stored, and/or the animal can be used to generate MAbs.

Following immunization, somatic cells with the potential for producing antibodies, specifically B lymphocytes (B cells), are selected for use in the MAb generating protocol. These cells may be obtained from biopsied spleens, lymph nodes, tonsils or adenoids, bone marrow aspirates or biopsies, tissue biopsies from mucosal organs like lung or GI tract, or from circulating blood. The antibody-producing B lymphocytes from the immunized animal or immune human are then fused with cells of an immortal myeloma cell, generally one of the same species as the animal that was immunized or human or human/mouse chimeric cells. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). Any one of a number of myeloma cells may be used, as are known to those of skill in the art. HMMA2.5 cells or MFP-2 cells are particularly useful examples of such cells.

Methods for generating hybrids of antibody-producing spleen or lymph node cells and myeloma cells usually comprise mixing somatic cells with myeloma cells in a 2:1 proportion, though the proportion may vary from about 20:1 to about 1:1, respectively, in the presence of an agent or agents (chemical or electrical) that promote the fusion of cell membranes. In some cases, transformation of human B cells with Epstein Barr virus (EBV) as an initial step increases the size of the B cells, enhancing fusion with the relatively large-sized myeloma cells. Transformation efficiency by EBV is enhanced by using CpG and a Chk2 inhibitor drug in the transforming medium. Alternatively, human B cells can be activated by co-culture with transfected cell lines expressing CD40 Ligand (CD154) in medium containing additional soluble factors, such as IL-21 and human B cell Activating Factor (BAFF), a Type II member of the TNF superfamily. Fusion methods using Sendai virus have been described by Kohler and Milstein (1975; 1976), and those using polyethylene glycol (PEG), such as 37% (v/v) PEG, by Gefter et al. (1977). The use of electrically induced fusion methods also is appropriate (Goding, pp. 71-74, 1986) and there are processes for better efficiency (Yu et al., 2008). Fusion procedures usually produce viable hybrids at low frequencies, about 1×10⁻⁶ to 1×10⁻⁸, but with optimized procedures one can achieve fusion efficiencies close to 1 in 200 (Yu et al., 2008). However, relatively low efficiency of fusion does not pose a problem, as the viable, fused hybrids are differentiated from the parental, infused cells (particularly the infused myeloma cells that would normally continue to divide indefinitely) by culturing in a selective medium. The selective medium is generally one that contains an agent that blocks the de novo synthesis of nucleotides in the tissue culture medium. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the medium is supplemented with hypoxanthine and thymidine as a source of nucleotides (HAT medium). Where azaserine is used, the medium is supplemented with hypoxanthine. Ouabain is added if the B cell source is an EBV-transformed human B cell line, in order to eliminate EBV-transformed lines that have not fused to the myeloma.

The preferred selection medium is HAT or HAT with ouabain. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B cells. When the source of B cells used for fusion is a line of EBV-transformed B cells, as here, ouabain may also be used for drug selection of hybrids as EBV-transformed B cells are susceptible to drug killing, whereas the myeloma partner used is chosen to be ouabain resistant.

Culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays dot immunobinding assays, and the like. The selected hybridomas are then serially diluted or single-cell sorted by flow cytometric sorting and cloned into individual antibody-producing cell lines, which clones can then be propagated indefinitely to provide mAbs. The cell lines may be exploited for MAb production in two basic ways. A sample of the hybridoma can be injected (often into the peritoneal cavity) into an animal (e.g., a mouse). Optionally, the animals are primed with a hydrocarbon, especially oils such as pristane (tetramethylpentadecane) prior to injection. When human hybridomas are used in this way, it is optimal to inject immunocompromised mice, such as SCID mice, to prevent tumor rejection. The injected animal develops tumors secreting the specific monoclonal antibody produced by the fused cell hybrid. The body fluids of the animal, such as serum or ascites fluid, can then be tapped to provide MAbs in high concentration. The individual cell lines could also be cultured in vitro, where the MAbs are naturally secreted into the culture medium from which they can be readily obtained in high concentrations. Alternatively, human hybridoma cells lines can be used in vitro to produce immunoglobulins in cell supernatant. The cell lines can be adapted for growth in serum-free medium to optimize the ability to recover human monoclonal immunoglobulins of high purity.

MAbs produced by either means may be further purified, if desired, using filtration, centrifugation and various chromatographic methods such as FPLC or affinity chromatography. Fragments of the monoclonal antibodies of the disclosure can be obtained from the purified monoclonal antibodies by methods which include digestion with enzymes, such as pepsin or papain, and/or by cleavage of disulfide bonds by chemical reduction. Alternatively, monoclonal antibody fragments encompassed by the present disclosure can be synthesized using an automated peptide synthesizer.

It also is contemplated that a molecular cloning approach may be used to generate monoclonal antibodies. Single B cells labelled with the antigen of interest can be sorted physically using paramagnetic bead selection or flow cytometric sorting, then RNA can be isolated from the single cells and antibody genes amplified by RT-PCR. Alternatively, antigen-specific bulk sorted populations of cells can be segregated into microvesicles and the matched heavy and light chain variable genes recovered from single cells using physical linkage of heavy and light chain amplicons, or common barcoding of heavy and light chain genes from a vesicle. Matched heavy and light chain genes form single cells also can be obtained from populations of antigen specific B cells by treating cells with cell-penetrating nanoparticles bearing RT-PCR primers and barcodes for marking transcripts with one barcode per cell. The antibody variable genes also can be isolated by RNA extraction of a hybridoma line and the antibody genes obtained by RT-PCR and cloned into an immunoglobulin expression vector. Alternatively, combinatorial immunoglobulin phagemid libraries are prepared from RNA isolated from the cell lines and phagemids expressing appropriate antibodies are selected by panning using tau antigens. The advantages of this approach over conventional hybridoma techniques are that approximately 10⁴ times as many antibodies can be produced and screened in a single round, and that new specificities are generated by H and L chain combination which further increases the chance of finding appropriate antibodies.

Other U.S. patents, each incorporated herein by reference, that teach the production of antibodies useful in the present disclosure include U.S. Pat. No. 5,565,332, which describes the production of chimeric antibodies using a combinatorial approach; U.S. Pat. No. 4,816,567 which describes recombinant immunoglobulin preparations; and U.S. Pat. No. 4,867,973 which describes antibody-therapeutic agent conjugates.

B. Antibodies of the Present Disclosure

Antibodies according to the present disclosure may be defined, in the first instance, by their binding specificity. Those of skill in the art, by assessing the binding specificity/affinity of a given antibody using techniques well known to those of skill in the art, can determine whether such antibodies fall within the scope of the instant claims. For example, the epitope to which a given antibody bind may consist of a single contiguous sequence of 4 or more (e.g., 4, 5, 6, 7, 8, 9, 10, 11, 12, 13, 14, 15, 16, 17, 18, 19, 20) amino acids located within the antigen molecule (e.g., a linear epitope in a domain). Alternatively, the epitope may consist of a plurality of non-contiguous amino acids (or amino acid sequences) located within the antigen molecule (e.g., a conformational epitope).

Various techniques known to persons of ordinary skill in the art can be used to determine whether an antibody “interacts with one or more amino acids” within a polypeptide or protein. Exemplary techniques include, for example, routine cross-blocking assays, such as that described in Antibodies, Harlow and Lane (Cold Spring Harbor Press, Cold Spring Harbor, N.Y.). Cross-blocking can be measured in various binding assays such as ELISA, biolayer interferometry, or surface plasmon resonance. Other methods include alanine scanning mutational analysis, peptide blot analysis (Reineke (2004) Methods Mol. Biol. 248: 443-63), peptide cleavage analysis, high-resolution electron microscopy techniques using single particle reconstruction, cryoEM, or tomography, crystallographic studies and NMR analysis. In addition, methods such as epitope excision, epitope extraction and chemical modification of antigens can be employed (Tomer (2000) Prot. Sci. 9: 487-496). Another method that can be used to identify the amino acids within a polypeptide with which an antibody interacts is hydrogen/deuterium exchange detected by mass spectrometry. In general terms, the hydrogen/deuterium exchange method involves deuterium-labeling the protein of interest, followed by binding the antibody to the deuterium-labeled protein. Next, the protein/antibody complex is transferred to water and exchangeable protons within amino acids that are protected by the antibody complex undergo deuterium-to-hydrogen back-exchange at a slower rate than exchangeable protons within amino acids that are not part of the interface. As a result, amino acids that form part of the protein/antibody interface may retain deuterium and therefore exhibit relatively higher mass compared to amino acids not included in the interface. After dissociation of the antibody, the target protein is subjected to protease cleavage and mass spectrometry analysis, thereby revealing the deuterium-labeled residues which correspond to the specific amino acids with which the antibody interacts. See, e.g., Ehring (1999) Analytical Biochemistry 267: 252-259; Engen and Smith (2001) Anal. Chem. 73: 256A-265A.

The term “epitope” refers to a site on an antigen to which B and/or T cells respond. B-cell epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation.

Modification-Assisted Profiling (MAP), also known as Antigen Structure-based Antibody Profiling (ASAP) is a method that categorizes large numbers of monoclonal antibodies (mAbs) directed against the same antigen according to the similarities of the binding profile of each antibody to chemically or enzymatically modified antigen surfaces (see US 2004/0101920, herein specifically incorporated by reference in its entirety). Each category may reflect a unique epitope either distinctly different from or partially overlapping with epitope represented by another category. This technology allows rapid filtering of genetically identical antibodies, such that characterization can be focused on genetically distinct antibodies. When applied to hybridoma screening, MAP may facilitate identification of rare hybridoma clones that produce mAbs having the desired characteristics. MAP may be used to sort the antibodies of the disclosure into groups of antibodies binding different epitopes.

The present disclosure includes antibodies that may bind to the same epitope, or a portion of the epitope. Likewise, the present disclosure also includes antibodies that compete for binding to a target or a fragment thereof with any of the specific exemplary antibodies described herein. One can easily determine whether an antibody binds to the same epitope as, or competes for binding with, a reference antibody by using routine methods known in the art. For example, to determine if a test antibody binds to the same epitope as a reference, the reference antibody is allowed to bind to target under saturating conditions. Next, the ability of a test antibody to bind to the target molecule is assessed. If the test antibody is able to bind to the target molecule following saturation binding with the reference antibody, it can be concluded that the test antibody binds to a different epitope than the reference antibody. On the other hand, if the test antibody is not able to bind to the target molecule following saturation binding with the reference antibody, then the test antibody may bind to the same epitope as the epitope bound by the reference antibody.

To determine if an antibody competes for binding with a reference anti-tau antibody, the above-described binding methodology is performed in two orientations: In a first orientation, the reference antibody is allowed to bind to the tau antigen under saturating conditions followed by assessment of binding of the test antibody to the tau antigen. In a second orientation, the test antibody is allowed to bind to the tau antigen under saturating conditions followed by assessment of binding of the reference antibody to the tau antigen. If, in both orientations, only the first (saturating) antibody is capable of binding to tau, then it is concluded that the test antibody and the reference antibody compete for binding to tau. As will be appreciated by a person of ordinary skill in the art, an antibody that competes for binding with a reference antibody may not necessarily bind to the identical epitope as the reference antibody but may sterically block binding of the reference antibody by binding an overlapping or adjacent epitope.

Two antibodies bind to the same or overlapping epitope if each competitively inhibits (blocks) binding of the other to the antigen. That is, a 1-, 5-, 10-, 20- or 100-fold excess of one antibody inhibits binding of the other by at least 50% but preferably 75%, 90% or even 99% as measured in a competitive binding assay (see, e.g., Junghans et al., Cancer Res. 1990 50:1495-1502). Alternatively, two antibodies have the same epitope if essentially all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Additional routine experimentation (e.g., peptide mutation and binding analyses) can then be carried out to confirm whether the observed lack of binding of the test antibody is in fact due to binding to the same epitope as the reference antibody or if steric blocking (or another phenomenon) is responsible for the lack of observed binding. Experiments of this sort can be performed using ELISA, RIA, surface plasmon resonance, flow cytometry or any other quantitative or qualitative antibody-binding assay available in the art. Structural studies with EM or crystallography also can demonstrate whether or not two antibodies that compete for binding recognize the same epitope.

C. Engineering of Antibody Sequences

In various embodiments, one may choose to engineer sequences of the identified antibodies for a variety of reasons, such as improved expression, improved cross-reactivity or diminished off-target binding. Modified antibodies may be made by any technique known to those of skill in the art, including expression through standard molecular biological techniques, or the chemical synthesis of polypeptides. Methods for recombinant expression are addressed elsewhere in this document. The following is a general discussion of relevant goals techniques for antibody engineering.

Hybridomas may be cultured, then cells lysed, and total RNA extracted. Random hexamers may be used with RT to generate cDNA copies of RNA, and then PCR performed using a multiplex mixture of PCR primers expected to amplify all human variable gene sequences. PCR product can be cloned into pGEM-T Easy vector, then sequenced by automated DNA sequencing using standard vector primers. Assay of binding and neutralization may be performed using antibodies collected from hybridoma supernatants and purified by FPLC, using Protein G columns.

Recombinant full-length IgG antibodies can be generated by subcloning heavy and light chain Fv DNAs from the cloning vector into an IgG plasmid vector, transfected into 293 (e.g., Freestyle) cells or CHO cells, and antibodies can be collected and purified from the 293 or CHO cell supernatant. Other appropriate host cells systems include bacteria, such as E. coli, insect cells (S2, Sf9, Sf29, High Five), plant cells (e.g., tobacco, with or without engineering for human-like glycans), algae, or in a variety of non-human transgenic contexts, such as mice, rats, goats or cows.

Expression of nucleic acids encoding antibodies, both for the purpose of subsequent antibody purification, and for immunization of a host, is also contemplated. Antibody coding sequences can be RNA, such as native RNA or modified RNA. Modified RNA contemplates certain chemical modifications that confer increased stability and low immunogenicity to mRNAs, thereby facilitating expression of therapeutically important proteins. For instance, N1-methyl-pseudouridine (N1mΨ) outperforms several other nucleoside modifications and their combinations in terms of translation capacity. In addition to turning off the immune/eIF2α phosphorylation-dependent inhibition of translation, incorporated N1mΨ nucleotides dramatically alter the dynamics of the translation process by increasing ribosome pausing and density on the mRNA. Increased ribosome loading of modified mRNAs renders them more permissive for initiation by favoring either ribosome recycling on the same mRNA or de novo ribosome recruitment. Such modifications could be used to enhance antibody expression in vivo following inoculation with RNA. The RNA, whether native or modified, may be delivered as naked RNA or in a delivery vehicle, such as a lipid nanoparticle.

Alternatively, DNA encoding the antibody may be employed for the same purposes. The DNA is included in an expression cassette comprising a promoter active in the host cell for which it is designed. The expression cassette is advantageously included in a replicable vector, such as a conventional plasmid or minivector. Vectors include viral vectors, such as poxviruses, adenoviruses, herpesviruses, adeno-associated viruses, and lentiviruses are contemplated. Replicons encoding antibody genes such as alphavirus replicons based on VEE virus or Sindbis virus are also contemplated. Delivery of such vectors can be performed by needle through intramuscular, subcutaneous, or intradermal routes, or by transcutaneous electroporation when in vivo expression is desired.

The rapid availability of antibody produced in the same host cell and cell culture process as the final cGMP manufacturing process has the potential to reduce the duration of process development programs. Lonza has developed a generic method using pooled transfectants grown in CDACF medium, for the rapid production of small quantities (up to 50 g) of antibodies in CHO cells. Although slightly slower than a true transient system, the advantages include a higher product concentration and use of the same host and process as the production cell line. Example of growth and productivity of GS-CHO pools, expressing a model antibody, in a disposable bioreactor: in a disposable bag bioreactor culture (5 L working volume) operated in fed-batch mode, a harvest antibody concentration of 2 g/L was achieved within 9 weeks of transfection.

Antibody molecules will comprise fragments (such as F(ab′), F(ab′)₂) that are produced, for example, by the proteolytic cleavage of the mAbs, or single-chain immunoglobulins producible, for example, via recombinant means. F(ab′) antibody derivatives are monovalent, while F(ab′)₂ antibody derivatives are bivalent. In one embodiment, such fragments can be combined with one another, or with other antibody fragments or receptor ligands to form “chimeric” binding molecules. Significantly, such chimeric molecules may contain substituents capable of binding to different epitopes of the same molecule.

In related embodiments, the antibody is a derivative of the disclosed antibodies, e.g., an antibody comprising the CDR sequences identical to those in the disclosed antibodies (e.g., a chimeric, or CDR-grafted antibody). Alternatively, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5 ±1), alanine (−0.5), and glycine (0); hydrophobic, aromatic amino acids: tryptophan (−3.4), phenylalanine (−2.5), and tyrosine (−2.3).

It is understood that an amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG₁ can increase antibody dependent cell cytotoxicity, switching to class A can improve tissue distribution, and switching to class M can improve valency.

Alternatively or additionally, it may be useful to combine amino acid modifications with one or more further amino acid modifications that alter C1q binding and/or the complement dependent cytotoxicity (CDC) function of the Fc region of an IL-23p19 binding molecule. The binding polypeptide of particular interest may be one that binds to C1q and displays complement dependent cytotoxicity. Polypeptides with pre-existing C1q binding activity, optionally further having the ability to mediate CDC may be modified such that one or both of these activities are enhanced Amino acid modifications that alter C1q and/or modify its complement dependent cytotoxicity function are described, for example, in WO/0042072, which is hereby incorporated by reference.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

FcRn binding. Fc mutations can also be introduced and engineered to alter their interaction with the neonatal Fc receptor (FcRn) and improve their pharmacokinetic properties. A collection of human Fc variants with improved binding to the FcRn have been described (Shields et al., (2001). High resolution mapping of the binding site on human IgG1 for FcγRI, FcγRII, FcγRIII, and FcRn and design of IgG1 variants with improved binding to the FcγR, (J. Biol. Chem. 276:6591-6604). A number of methods are known that can result in increased half-life (Kuo and Aveson, 2011), including amino acid modifications may be generated through techniques including alanine scanning mutagenesis, random mutagenesis and screening to assess the binding to the neonatal Fc receptor (FcRn) and/or the in vivo behavior. Computational strategies followed by mutagenesis may also be used to select one of amino acid mutations to mutate.

The present disclosure therefore provides a variant of an antigen binding protein with optimized binding to FcRn. In a particular embodiment, the said variant of an antigen binding protein comprises at least one amino acid modification in the Fc region of said antigen binding protein, wherein said modification is selected from the group consisting of 226, 227, 228, 230, 231, 233, 234, 239, 241, 243, 246, 250, 252, 256, 259, 264, 265, 267, 269, 270, 276, 284, 285, 288, 289, 290, 291, 292, 294, 297, 298, 299, 301, 302, 303, 305, 307, 308, 309, 311, 315, 317, 320, 322, 325, 327, 330, 332, 334, 335, 338, 340, 342, 343, 345, 347, 350, 352, 354, 355, 356, 359, 360, 361, 362, 369, 370, 371, 375, 378, 380, 382, 384, 385, 386, 387, 389, 390, 392, 393, 394, 395, 396, 397, 398, 399, 400, 401 403, 404, 408, 411, 412, 414, 415, 416, 418, 419, 420, 421, 422, 424, 426, 428, 433, 434, 438, 439, 440, 443, 444, 445, 446 and 447 of the Fc region as compared to said parent polypeptide, wherein the numbering of the amino acids in the Fc region is that of the EU index in Kabat. In a further aspect of the disclosure the modifications are M252Y/S254T/T256E.

Additionally, various publications describe methods for obtaining physiologically active molecules whose half-lives are modified, see for example Kontermann (2009) either by introducing an FcRn-binding polypeptide into the molecules or by fusing the molecules with antibodies whose FcRn-binding affinities are preserved but affinities for other Fc receptors have been greatly reduced or fusing with FcRn binding domains of antibodies.

Derivatized antibodies may be used to alter the half-lives (e.g., serum half-lives) of parental antibodies in a mammal, particularly a human Such alterations may result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the antibodies of the present disclosure or fragments thereof in a mammal, preferably a human, results in a higher serum titer of said antibodies or antibody fragments in the mammal, and thus reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting or adding) amino acid residues identified as involved in the interaction between the Fc domain and the FcRn receptor.

Beltramello et al. (2010) previously reported the modification of neutralizing mAbs, due to their tendency to enhance dengue virus infection, by generating in which leucine residues at positions 1.3 and 1.2 of CH2 domain (according to the IMGT unique numbering for C-domain) were substituted with alanine residues. This modification, also known as “LALA” mutation, abolishes antibody binding to FcγRI, FcγRII and FcγRIIIa, as described by Hessell et al. (2007). The variant and unmodified recombinant mAbs were compared for their capacity to neutralize and enhance infection by the four dengue virus serotypes. LALA variants retained the same neutralizing activity as unmodified mAb, but were completely devoid of enhancing activity. LALA mutations of this nature are therefore contemplated in the context of the presently disclosed antibodies.

Altered Glycosylation. A particular embodiment of the present disclosure is an isolated monoclonal antibody, or antigen binding fragment thereof, containing a substantially homogeneous glycan without sialic acid, galactose, or fucose. The monoclonal antibody comprises a heavy chain variable region and a light chain variable region, both of which may be attached to heavy chain or light chain constant regions respectively. The aforementioned substantially homogeneous glycan may be covalently attached to the heavy chain constant region.

Another embodiment of the present disclosure comprises a mAb with a novel Fc glycosylation pattern. The isolated monoclonal antibody, or antigen binding fragment thereof, is present in a substantially homogenous composition represented by the GNGN or G1/G2 glycoform. Fc glycosylation plays a significant role in the properties of therapeutic mAbs. This embodiment of the present disclosure with homogenous glycans lacking a core fucose, showed increased protection against specific viruses by a factor greater than two-fold. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, comprising a substantially homogenous composition represented by the GNGN or G1/G2 glycoform exhibits increased binding affinity for Fc gamma RI and Fc gamma RIII compared to the same antibody without the substantially homogeneous GNGN glycoform and with G0, G1F, G2F, GNF, GNGNF or GNGNFX containing glycoforms. In one embodiment of the present disclosure, the antibody dissociates from Fc gamma RI with a Kd of 1×10⁻⁸ M or less and from Fc gamma RIII with a Kd of 1×10⁻⁷ M or less.

Glycosylation of an Fc region is typically either N-linked or O-linked. N-linked refers to the attachment of the carbohydrate moiety to the side chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the antibody is expressed in cells that express beta (1,4)-N-acetylglucosaminyltransferase III (GnT III), such that GnT III adds GlcNAc to the IL-23p19 antibody. Methods for producing antibodies in such a fashion are provided in WO/9954342, WO/03011878, U.S. Patent Publication 20030003097A1, and Umana et al., Nature Biotechnology, 17:176-180, February 1999. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express recombinant monoclonal antibodies.

Elimination of monoclonal antibody protein sequence liabilities. It is possible to engineer the antibody variable gene sequences obtained from human B cells to enhance their manufacturability and safety. Potential protein sequence liabilities can be identified by searching for sequence motifs associated with sites containing:

1) Unpaired Cys residues,

2) N-linked glycosylation,

3) Asn deamidation,

4) Asp isomerization,

5) SYE truncation,

6) Met oxidation,

7) Trp oxidation,

8) N-terminal glutamate,

9) Integrin binding,

10) CD11c/CD18 binding, or

11) Fragmentation

Such motifs can be eliminated by altering the synthetic gene for the cDNA encoding recombinant antibodies.

Protein engineering efforts in the field of development of therapeutic antibodies clearly reveal that certain sequences or residues are associated with solubility differences (Fernandez-Escamilla et al., Nature Biotech., 22 (10), 1302-1306, 2004; Chennamsetty et al., PNAS, 106 (29), 11937-11942, 2009; Voynov et al., Biocon. Chem., 21(2), 385-392, 2010) Evidence from solubility-altering mutations in the literature indicate that some hydrophilic residues such as aspartic acid, glutamic acid, and serine contribute significantly more favorably to protein solubility than other hydrophilic residues, such as asparagine, glutamine, threonine, lysine, and arginine.

Stability. Antibodies can be engineered for enhanced biophysical properties. One can use elevated temperature to unfold antibodies to determine relative stability, using average apparent melting temperatures. Differential Scanning Calorimetry (DSC) measures the heat capacity, Cₚ, of a molecule (the heat required to warm it, per degree) as a function of temperature. One can use DSC to study the thermal stability of antibodies. DSC data for mAbs is particularly interesting because it sometimes resolves the unfolding of individual domains within the mAb structure, producing up to three peaks in the thermogram (from unfolding of the Fab, C_{H}2, and C_{H}3 domains). Typically unfolding of the Fab domain produces the strongest peak. The DSC profiles and relative stability of the Fc portion show characteristic differences for the human IgG₁, IgG₂, IgG₃, and IgG₄ subclasses (Garber and Demarest, Biochem. Biophys. Res. Commun. 355, 751-757, 2007). One also can determine average apparent melting temperature using circular dichroism (CD), performed with a CD spectrometer. Far-UV CD spectra will be measured for antibodies in the range of 200 to 260 nm at increments of 0.5 nm. The final spectra can be determined as averages of 20 accumulations. Residue ellipticity values can be calculated after background subtraction. Thermal unfolding of antibodies (0.1 mg/mL) can be monitored at 235 nm from 25-95° C. and a heating rate of 1° C./min One can use dynamic light scattering (DLS) to assess for propensity for aggregation. DLS is used to characterize size of various particles including proteins. If the system is not disperse in size, the mean effective diameter of the particles can be determined. This measurement depends on the size of the particle core, the size of surface structures, and particle concentration. Since DLS essentially measures fluctuations in scattered light intensity due to particles, the diffusion coefficient of the particles can be determined. DLS software in commercial DLA instruments displays the particle population at different diameters. Stability studies can be done conveniently using DLS. DLS measurements of a sample can show whether the particles aggregate over time or with temperature variation by determining whether the hydrodynamic radius of the particle increases. If particles aggregate, one can see a larger population of particles with a larger radius. Stability depending on temperature can be analyzed by controlling the temperature in situ. Capillary electrophoresis (CE) techniques include proven methodologies for determining features of antibody stability. One can use an iCE approach to resolve antibody protein charge variants due to deamidation, C-terminal lysines, sialylation, oxidation, glycosylation, and any other change to the protein that can result in a change in pI of the protein. Each of the expressed antibody proteins can be evaluated by high throughput, free solution isoelectric focusing (IEF) in a capillary column (cIEF), using a Protein Simple Maurice instrument. Whole-column UV absorption detection can be performed every 30 seconds for real time monitoring of molecules focusing at the isoelectric points (pIs). This approach combines the high resolution of traditional gel IEF with the advantages of quantitation and automation found in column-based separations while eliminating the need for a mobilization step. The technique yields reproducible, quantitative analysis of identity, purity, and heterogeneity profiles for the expressed antibodies. The results identify charge heterogeneity and molecular sizing on the antibodies, with both absorbance and native fluorescence detection modes and with sensitivity of detection down to 0.7 μg/mL.

Solubility. One can determine the intrinsic solubility score of antibody sequences. The intrinsic solubility scores can be calculated using CamSol Intrinsic (Sormanni et al., J Mol Biol 427, 478-490, 2015). The amino acid sequences for residues 95-102 (Kabat numbering) in HCDR3 of each antibody fragment such as a scFv can be evaluated via the online program to calculate the solubility scores. One also can determine solubility using laboratory techniques. Various techniques exist, including addition of lyophilized protein to a solution until the solution becomes saturated and the solubility limit is reached, or concentration by ultrafiltration in a microconcentrator with a suitable molecular weight cut-off. The most straightforward method is induction of amorphous precipitation, which measures protein solubility using a method involving protein precipitation using ammonium sulfate (Trevino et al., J Mol Biol, 366: 449-460, 2007). Ammonium sulfate precipitation gives quick and accurate information on relative solubility values. Ammonium sulfate precipitation produces precipitated solutions with well-defined aqueous and solid phases and requires relatively small amounts of protein. Solubility measurements performed using induction of amorphous precipitation by ammonium sulfate also can be done easily at different pH values. Protein solubility is highly pH dependent, and pH is considered the most important extrinsic factor that affects solubility.

Autoreactivity. Generally, it is thought that autoreactive clones should be eliminated during ontogeny by negative selection, however it has become clear that many human naturally occurring antibodies with autoreactive properties persist in adult mature repertoires, and the autoreactivity may enhance the function of many antibodies. It has been noted that HCDR3 loops in antibodies during early B cell development are often rich in positive charge and exhibit autoreactive patterns (Wardemann et al., Science 301, 1374-1377, 2003). One can test a given antibody for autoreactivity by assessing the level of binding to human origin cells in microscopy (using adherent HeLa or HEp-2 epithelial cells) and flow cytometric cell surface staining (using suspension Jurkat T cells and 293S human embryonic kidney cells). Autoreactivity also can be surveyed using assessment of binding to tissues in tissue arrays.

Preferred residues (“Human Likeness”). B cell repertoire deep sequencing of human B cells from blood donors is being performed on a wide scale in many recent studies. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

D. Single Chain Antibodies

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine and glycine. However, other residues can function as well. Tang et al. (1996) used phage display as a means of rapidly selecting tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 5×10⁶ different members) was displayed on filamentous phage and subjected to affinity selection with hapten. The population of selected variants exhibited significant increases in binding activity but retained considerable sequence diversity. Screening 1054 individual variants subsequently yielded a catalytically active scFv that was produced efficiently in soluble form. Sequence analysis revealed a conserved proline in the linker two residues after the V_{H} C terminus and an abundance of arginines and prolines at other positions as the only common features of the selected tethers.

The recombinant antibodies of the present disclosure may also involve sequences or moieties that permit dimerization or multimerization of the receptors. Such sequences include those derived from IgA, which permit formation of multimers in conjunction with the J-chain. Another multimerization domain is the Gal4 dimerization domain. In other embodiments, the chains may be modified with agents such as biotin/avidin, which permit the combination of two antibodies.

In a separate embodiment, a single-chain antibody can be created by joining receptor light and heavy chains using a non-peptide linker or chemical unit. Generally, the light and heavy chains will be produced in distinct cells, purified, and subsequently linked together in an appropriate fashion (i.e., the N-terminus of the heavy chain being attached to the C-terminus of the light chain via an appropriate chemical bridge).

Cross-linking reagents are used to form molecular bridges that tie functional groups of two different molecules, e.g., a stabilizing and coagulating agent. However, it is contemplated that dimers or multimers of the same analog or heteromeric complexes comprised of different analogs can be created. To link two different compounds in a step-wise manner, hetero-bifunctional cross-linkers can be used that eliminate unwanted homopolymer formation.

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhydryl group) of the other protein (e.g., the selective agent).

It is preferred that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1987). The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Particular uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

E. Multispecific Antibodies

In certain embodiments, antibodies of the present disclosure are bispecific or multispecific. Bispecific antibodies are antibodies that have binding specificities for at least two different epitopes. Exemplary bispecific antibodies may bind to two different epitopes of a single antigen. Other such antibodies may combine a first antigen binding site with a binding site for a second antigen. Alternatively, an anti-pathogen arm may be combined with an arm that binds to a triggering molecule on a leukocyte, such as a T-cell receptor molecule (e.g., CD3), or Fc receptors for IgG (FcγR), such as FcγRI (CD64), FcγRII (CD32) and Fc gamma RIII (CD16), so as to focus and localize cellular defense mechanisms to the infected cell. Bispecific antibodies may also be used to localize cytotoxic agents to infected cells. These antibodies possess a pathogen-binding arm and an arm that binds the cytotoxic agent (e.g., saporin, anti-interferon-α, vinca alkaloid, ricin A chain, methotrexate or radioactive isotope hapten). Bispecific antibodies can be prepared as full-length antibodies or antibody fragments (e.g., F(ab′)₂ bispecific antibodies). WO 96/16673 describes a bispecific anti-ErbB2/anti-Fc gamma RIII antibody and U.S. Pat. No. 5,837,234 discloses a bispecific anti-ErbB2/anti-Fc gamma RI antibody. A bispecific anti-ErbB2/Fc alpha antibody is shown in WO98/02463. U.S. Pat. No. 5,821,337 teaches a bispecific anti-ErbB2/anti-CD3 antibody.

Methods for making bispecific antibodies are known in the art. Traditional production of full length bispecific antibodies is based on the co-expression of two immunoglobulin heavy chain-light chain pairs, where the two chains have different specificities (Millstein et al., Nature, 305:537-539 (1983)). Because of the random assortment of immunoglobulin heavy and light chains, these hybridomas (quadromas) produce a potential mixture of ten different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J., 10:3655-3659 (1991).

According to a different approach, antibody variable regions with the desired binding specificities (antibody-antigen combining sites) are fused to immunoglobulin constant domain sequences. Preferably, the fusion is with an Ig heavy chain constant domain, comprising at least part of the hinge, C_{H2}, and C_{H3} regions. It is preferred to have the first heavy-chain constant region (C_{H1}) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant effect on the yield of the desired chain combination.

In a particular embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods Enzymol., 121:210 (1986).

According to another approach described in U.S. Pat. No. 5,731,168, the interface between a pair of antibody molecules can be engineered to maximize the percentage of heterodimers that are recovered from recombinant cell culture. The preferred interface comprises at least a part of the C_{H3} domain In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science, 229: 81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Techniques exist that facilitate the direct recovery of Fab′-SH fragments from E. coli, which can be chemically coupled to form bispecific antibodies. Shalaby et al., J. Exp. Med., 175: 217-225 (1992) describe the production of a humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described (Merchant et al., Nat. Biotechnol. 16, 677-681 (1998). doi:10.1038/nbt0798-677pmid:9661204). For example, bispecific antibodies have been produced using leucine zippers (Kostelny et al., J. Immunol., 148(5):1547-1553, 1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA, 90:6444-6448 (1993) has provided an alternative mechanism for making bispecific antibody fragments. The fragments comprise a V_{H} connected to a V_{L} by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

In a particular embodiment, a bispecific or multispecific antibody may be formed as a DOCK-AND-LOCK™ (DNL™) complex (see, e.g., U.S. Pat. Nos. 7,521,056; 7,527,787; 7,534,866; 7,550,143 and 7,666,400, the Examples section of each of which is incorporated herein by reference.) Generally, the technique takes advantage of the specific and high-affinity binding interactions that occur between a dimerization and docking domain (DDD) sequence of the regulatory (R) subunits of cAMP-dependent protein kinase (PKA) and an anchor domain (AD) sequence derived from any of a variety of AKAP proteins (Baillie et al., FEBS Letters. 2005; 579: 3264; Wong and Scott, Nat. Rev. Mol. Cell Biol. 2004; 5: 959). The DDD and AD peptides may be attached to any protein, peptide or other molecule. Because the DDD sequences spontaneously dimerize and bind to the AD sequence, the technique allows the formation of complexes between any selected molecules that may be attached to DDD or AD sequences.

Antibodies with more than two valencies are contemplated. For example, trispecific antibodies can be prepared (Tutt et al., J. Immunol. 147: 60, 1991; Xu et al., Science, 358(6359):85-90, 2017). A multivalent antibody may be internalized (and/or catabolized) faster than a bivalent antibody by a cell expressing an antigen to which the antibodies bind. The antibodies of the present disclosure can be multivalent antibodies with three or more antigen binding sites (e.g., tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable regions. For instance, the polypeptide chain(s) may comprise VD1-(X1)ₙ-VD2-(X2)ₙ-Fc, wherein VD1 is a first variable region, VD2 is a second variable region, Fc is one polypeptide chain of an Fc region, X1 and X2 represent an amino acid or polypeptide, and n is 0 or 1. For instance, the polypeptide chain(s) may comprise: VH—CH1-flexible linker-VH—CH1-Fc region chain; or VH—CH1-VH-—CH1-Fc region chain. The multivalent antibody herein preferably further comprises at least two (and preferably four) light chain variable region polypeptides. The multivalent antibody herein may, for instance, comprise from about two to about eight light chain variable region polypeptides. The light chain variable region polypeptides contemplated here comprise a light chain variable region and, optionally, further comprise a C_{L} domain.

Charge modifications are particularly useful in the context of a multispecific antibody, where amino acid substitutions in Fab molecules result in reducing the mispairing of light chains with non-matching heavy chains (Bence-Jones-type side products), which can occur in the production of Fab-based bi-/multispecific antigen binding molecules with a VH/VL exchange in one (or more, in case of molecules comprising more than two antigen-binding Fab molecules) of their binding arms (see also PCT publication no. WO 2015/150447, particularly the examples therein, incorporated herein by reference in its entirety).

Accordingly, in particular embodiments, an antibody comprised in the therapeutic agent comprises
(a) a first Fab molecule which specifically binds to a first antigen
(b) a second Fab molecule which specifically binds to a second antigen, and wherein the variable domains VL and VH of the Fab light chain and the Fab heavy chain are replaced by each other,
wherein the first antigen is an activating T cell antigen and the second antigen is a target cell antigen, or the first antigen is a target cell antigen and the second antigen is an activating T cell antigen; and
wherein
i) in the constant domain CL of the first Fab molecule under a) the amino acid at position 124 is substituted by a positively charged amino acid (numbering according to Kabat), and wherein in the constant domain CH1 of the first Fab molecule under a) the amino acid at position 147 or the amino acid at position 213 is substituted by a negatively charged amino acid (numbering according to Kabat EU index); or
ii) in the constant domain CL of the second Fab molecule under b) the amino acid at position 124 is substituted by a positively charged amino acid (numbering according to Kabat), and wherein in the constant domain CH1 of the second Fab molecule under b) the amino acid at position 147 or the amino acid at position 213 is substituted by a negatively charged amino acid (numbering according to Kabat EU index).

The antibody may not comprise both modifications mentioned under i) and ii). The constant domains CL and CH1 of the second Fab molecule are not replaced by each other (i.e., remain unexchanged).

In another embodiment of the antibody, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)), and in the constant domain CH1 of the first Fab molecule under a) the amino acid at position 147 or the amino acid at position 213 is substituted independently by glutamic acid (E), or aspartic acid (D) (numbering according to Kabat EU index).

In a further embodiment, in the constant domain CL of the first Fab molecule under a) the amino acid at position 124 is substituted independently by lysine (K), arginine (R) or histidine (H) (numbering according to Kabat), and in the constant domain CH1 of the first Fab molecule under a) the amino acid at position 147 is substituted independently by glutamic acid (E), or aspartic acid (D) (numbering according to Kabat EU index).

In a particular embodiment, in the constant domain CL of the first Fab molecule under a) the amino acid at position 124 is substituted independently by lysine (K), arginine (R) or histidine (H) (numbering according to Kabat) (in one preferred embodiment independently by lysine (K) or arginine (R)) and the amino acid at position 123 is substituted independently by lysine (K), arginine (R) or histidine (H) (numbering according to Kabat) (in one preferred embodiment independently by lysine (K) or arginine (R)), and in the constant domain CH1 of the first Fab molecule under a) the amino acid at position 147 is substituted independently by glutamic acid (E), or aspartic acid (D) (numbering according to Kabat EU index) and the amino acid at position 213 is substituted independently by glutamic acid (E), or aspartic acid (D) (numbering according to Kabat EU index).

In a more particular embodiment, in the constant domain CL of the first Fab molecule under a) the amino acid at position 124 is substituted by lysine (K) (numbering according to Kabat) and the amino acid at position 123 is substituted by lysine (K) or arginine (R) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

In an even more particular embodiment, in the constant domain CL of the first Fab molecule under a) the amino acid at position 124 is substituted by lysine (K) (numbering according to Kabat) and the amino acid at position 123 is substituted by arginine (R) (numbering according to Kabat), and in the constant domain CH1 of the first Fab molecule under a) the amino acid at position 147 is substituted by glutamic acid (E) (numbering according to Kabat EU index) and the amino acid at position 213 is substituted by glutamic acid (E) (numbering according to Kabat EU index).

F. Purification

In certain embodiments, the antibodies of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, antibodies and the like or by heat denaturation, followed by centrifugation; gel filtration, reverse phase, hydroxylapatite and affinity chromatography; and combinations of such and other techniques.

In purifying an antibody of the present disclosure, it may be desirable to express the polypeptide in a prokaryotic or eukaryotic expression system and extract the protein using denaturing conditions. The polypeptide may be purified from other cellular components using an affinity column, which binds to a tagged portion of the polypeptide. As is generally known in the art, it is believed that the order of conducting the various purification steps may be changed, or that certain steps may be omitted, and still result in a suitable method for the preparation of a substantially purified protein or peptide.

Commonly, complete antibodies are fractionated utilizing agents (i.e., protein A) that bind the Fc portion of the antibody. Alternatively, antigens may be used to simultaneously purify and select appropriate antibodies. Such methods often utilize the selection agent bound to a support, such as a column, filter or bead. The antibodies are bound to a support, contaminants removed (e.g., washed away), and the antibodies released by applying conditions (salt, heat, etc.).

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. Another method for assessing the purity of a fraction is to calculate the specific activity of the fraction, to compare it to the specific activity of the initial extract, and to thus calculate the degree of purity. The actual units used to represent the amount of activity will, of course, be dependent upon the particular assay technique chosen to follow the purification and whether or not the expressed protein or peptide exhibits a detectable activity.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

### III. TAU PEPTIDE EPITOPES

The inventor has identified a number of tau fragments they believe will prove useful in the production of antibodies for diagnosis and passive immunization, as well as active vaccines for the generation of therapeutic and/or protective immune responses in a subject. Both methodologies are disclosed elsewhere in this document, and those disclosures are incorporated here.

The peptides described herein represent sequences predicted to have unique exposure or structure in the setting of pathogenic forms of tau, and will thus represent important epitopes that will facilitate generation of effective vaccines and diagnostic immunoassays. Below the list of peptide epitopes is a detailed rationale to explain how the inventor used emergent information about tau structure and conformation to pick peptide sequences and non-natural modifications of said sequences to design effective antigens for both therapy and diagnosis. A summary of epitope designs disclosed are as follows:

R1/R3 hairpin

N-terminal portion of R1/R3 hairpin

C-terminal portion of R1/R3 hairpin

R2/R3 hairpin

N-terminal portion of R2/R3 hairpin

C-terminal portion of R2/R3 hairpin

R1/R2 hairpin

N-terminal portion of R1/R2 hairpin

C-terminal portion of R1/R2 hairpin

R3/R4 hairpin

N-terminal portion of R3/R4 hairpin

C-terminal portion of R3/R4 hairpin

R4 hairpin

N-terminal portion of R4 hairpin

C-terminal portion of R4 hairpin

Intervening fragments between the hairpins listed above

Tau274-311:
Fragments within Tau274-311 antigen
Truncations of Tau274-311 from both N-term and C-term ends
Conformational mutants of Tau274-311 antigen

Peptides are generally defined as amino acid polymers of nor more than about 100 residues. That maximum length may of course be smaller, such as 75 or less residues, 50 or less residues, or 37 residues. The minimum length of a peptide according to the the present disclosure is about 5 residues, but may be at least 6 residues, at least 7 residues, at least 10 residues or at least 20 residues in length. Ranges of minimum and maximum lengths may be produced by combining any two minimum and maximum sizes above, such as but not limited to 6 to 38 residues, 6 to 100 residues, 6 to 50 residues, 6-25 residues, 10-30 residues, and so forth. The peptides may also contain all L amino acids, all D amino acids, or a mix of L and D amino acids. The peptides may also contain amino acid analogs at specified or unspecified positions, or non-natural amino acids at unspecified positions. Also, conservative substitutions at specified positions are contemplated.

Whereas most vaccines are developed based on the concept of peptide sequences (defined below) of proteins of interest, here the inventor is using emergent knowledge of tau protein structure to identify short amino sequences that capture local secondary and even tertiary structures. This can in certain examples (detailed below, see trans-proline peptides) involve the creation of non-natural polypeptide sequences. Capturing antigens in specific conformational states creates specificity against pathogenic conformations of the tau protein. Thus, the definition of peptides herein captures structural information, in addition to pure amino acid sequence.

Peptides may be produced through expression using DNA constructs as would full length proteins. However, due to their shorter length, chemical synthesis may be employed. In organic chemistry, peptide synthesis is the production of peptides, compounds where multiple amino acids are linked via amide bonds, also known as peptide bonds. Peptides are chemically synthesized by the condensation reaction of the carboxyl group of one amino acid to the amino group of another. Protecting group strategies are usually necessary to prevent undesirable side reactions with the various amino acid side chains. Chemical peptide synthesis most commonly starts at the carboxyl end of the peptide (C-terminus), and proceeds toward the amino-terminus (N-terminus). Protein biosynthesis (long peptides) in living organisms occurs in the opposite direction.

The chemical synthesis of peptides can be carried out using classical solution-phase techniques, although these have been replaced in most research and development settings by solid-phase methods (see below). Solution-phase synthesis retains its usefulness in large-scale production of peptides for industrial purposes however.

Chemical synthesis facilitates the production of peptides which are difficult to express in bacteria, the incorporation of unnatural amino acids, peptide/protein backbone modification, and the synthesis of D-proteins, which consist of D-amino acids.

The established method for the production of synthetic peptides in the lab is known as solid-phase peptide synthesis (SPPS). Pioneered by Robert Bruce Merrifield, SPPS allows the rapid assembly of a peptide chain through successive reactions of amino acid derivatives on an insoluble porous support.

The solid support consists of small, polymeric resin beads functionalized with reactive groups (such as amine or hydroxyl groups) that link to the nascent peptide chain. Since the peptide remains covalently attached to the support throughout the synthesis, excess reagents and side products can be removed by washing and filtration. This approach circumvents the comparatively time-consuming isolation of the product peptide from solution after each reaction step, which would be required when using conventional solution-phase synthesis.

Each amino acid to be coupled to the peptide chain N-terminus must be protected on its N-terminus and side chain using appropriate protecting groups such as Boc (acid-labile) or Fmoc (base-labile), depending on the side chain and the protection strategy used.

The general SPPS procedure is one of repeated cycles of alternate N-terminal deprotection and coupling reactions. The resin can be washed between each steps. First an amino acid is coupled to the resin. Subsequently, the amine is deprotected, and then coupled with the free acid of the second amino acid. This cycle repeats until the desired sequence has been synthesized. SPPS cycles may also include capping steps which block the ends of unreacted amino acids from reacting. At the end of the synthesis, the crude peptide is cleaved from the solid support while simultaneously removing all protecting groups using a strong acid like trifluoroacetic acid or a nucleophile. The crude peptide can be precipitated from a non-polar solvent like diethyl ether in order to remove organic soluble by products. The crude peptide can be purified using reversed-phase HPLC. The purification process, especially of longer peptides can be challenging, because small amounts of several byproducts, which are very similar to the product, have to be removed. For this reason, so-called continuous chromatography processes such as MCSGP are increasingly being used in commercial settings to maximize the yield without sacrificing on purity levels.

SPPS is limited by reaction yields, and typically peptides and proteins in the range of 70 amino acids are pushing the limits of synthetic accessibility. Synthetic difficulty also is sequence dependent; typically, aggregation-prone sequences such as amyloids are difficult to make. Longer lengths can be accessed by using ligation approaches such as native chemical ligation, where two shorter fully deprotected synthetic peptides can be joined together in solution.

An important feature that has enabled the broad application of SPPS is the generation of extremely high yields in the coupling step. Highly efficient amide bond-formation conditions are required and an excess of each amino acid (between 2- and 10-fold) should be added. The minimization of amino acid racemization during coupling is also of vital importance to avoid epimerization in the final peptide product.

Amide bond formation between an amine and carboxylic acid is slow, and as such usually requires ‘coupling reagents’ or ‘activators’. Activation of the carboxyl group generally involves the formation of an ‘active ester’ in situ.

Carbodiimides such as dicyclohexylcarbodiimide (DCC) and diisopropylcarbodiimide (DIC) are frequently used for amide bond formation. The reaction proceeds via the formation of a highly reactive O-acylisourea. This reactive intermediate is attacked by the peptide N-terminal amine, forming a peptide bond. Formation of the O-acylisourea proceeds fastest in non-polar solvents such as dichloromethane.

DIC is particularly useful for SPPS since as a liquid it is easily dispensed, and the urea byproduct (N,N′-Diisopropylcarbodiimide is easily washed away. Conversely, the related carbodiimide 1-Ethyl-3-(3-dimethylaminopropyl)carbodiimide (EDC) is often used for solution-phase peptide couplings as its urea byproduct can be removed by washing during aqueous work-up.

Carbodiimide activation opens the possibility for racemization of the activated amino acid. Racemization can be circumvented with ‘racemization suppressing’ additives such as the triazoles 1-hydroxy-benzotriazole (HOBt), and 1-hydroxy-7-aza-benzotriazole (HOAt). These reagents attack the O-acylisourea intermediate to form an active ester, which subsequently reacts with the peptide to form the desired peptide bond. Ethyl cyanohydroxyiminoacetate (Oxyma), an additive for carbodiimide coupling, acts as an alternative to HOAt.

Some coupling reagents omit the carbodiimide completely and incorporate the HOAt/HOBt moiety as an aminium/uronium or phosphonium salt of a non-nucleophilic anion (tetrafluoroborate or hexafluorophosphate). Examples of aminium/uronium reagents include HATU (HOAt), HBTU/TBTU (HOBt) and HCTU (6-ClHOBt). HBTU and TBTU differ only in the choice of anion. Phosphonium reagents include PyBOP (HOBt) and PyAOP (HOAt).

These reagents form the same active ester species as the carbodiimide activation conditions but differ in the rate of the initial activation step, which is determined by nature of the carbon skeleton of the coupling reagent. Furthermore, aminium/uronium reagents are capable of reacting with the peptide N-terminus to form an inactive guanidino by-product, whereas as phosphonium reagents are not.

Solid supports for peptide synthesis are selected for physically stability, to permit the rapid filtration of liquids. Suitable supports are inert to reagents and solvents used during SPPS, although they must swell in the solvents used to allow for penetration of the reagents and allow for the attachment of the first amino acid.

Three primary types of solid supports are: gel-type supports, surface-type supports, and composites. Improvements to solid supports used for peptide synthesis enhance their ability to withstand the repeated use of TFA during the deprotection step of SPPS. Two primary resins are used, based on whether a C-terminal carboxylic acid or amide is desired. The Wang resin was, as of 1996, the most commonly used resin for peptides with C-terminal carboxylic acids.

Tau protein aggregation underlies neurodegeneration in Alzheimer's disease and related tauopathies. Tau has 6 isoforms, all of which can be found in pathological assemblies, or aggregates. Depending on splicing, these include 0, 1, or 2 N terminal domains, and 3 or 4 repeat domain sequences (Table A).

TABLE A
Nomenclature for 6 tau isoforms based on
inclusion/exclusion of N and R domains

| 0, 1, 2 N | 3, 4 R | Tau Nomenclature |
|---|---|---|
| 0 | 3 | 0N3R |
| 1 | 3 | 1N3R |
| 2 | 3 | 2N3R |
| 0 | 4 | 0N4R |
| 1 | 4 | 1N4R |
| 2 | 4 | 2N4R |

Recent work from the Center for Alzheimer's and Neurodegenerative Diseases indicates that tau protein, rather than being natively unstructured, in fact occupies distinct conformational ensembles. One class of structures is found in healthy controls and is relatively inert in biochemical assays of protein aggregation (herein termed Mᵢ). The second class is found in cases of tau pathology and represents a structure that is “seed-competent,” capable of self-assembly and acting as a conformational template to convert inert tau monomer to an aggregated state (herein termed Mₛ). In Mirbaha et al. (2018), the inventor used cross-linking with mass spectrometry (XL-MS) to determine that a specific intramolecular contact differentiates Mᵢ and Mₛ. This is associated with conformational change within the repeat domain (RD, comprised of 3 or 4 R sequences) of tau that exposes two amyloid-forming sequences, VQIINK (SEQ ID NO: 3) and VQIVYK (SEQ ID NO: 1). These sequences exist within predicted hairpin structures that exist at the junction of R2/R3 and R3/R4 of 4R tau. Additional predicted hairpins exist between the junction of R1/R2, R4/R′ (the sequences immediately C-terminal to the fourth repeat), and R1/R3 in the setting of 3R tau, which lacks the R2 region. Recent work indicates that these hairpin structures are destabilized in the setting of Mₛ. This predicts that sequences in and around these structures will represent important therapeutic and diagnostic targets.

Specific categories of peptides are set forth below:
1. Epitopes encompassing a non-natural amino acid at P301, specifically, trans-proline, will create specificity to detect and treat tau in pathological conformations. Structural biological studies published (Mirbaha et al., 2018), and unpublished (Drombosky, Biorxiv 2018) indicate that tau in a pathological configuration will exist with P301 in a trans-configuration. This predicts that epitopes which specifically encompass this structure will be excellent targets for immunotherapy (passive and active vaccine) as well as diagnostic tests to be used in spinal fluid or blood. To encompass adequate sections of protein the inventor predicts aa 294-311 will be most effective.
2. Epitopes encompassing hairpin structures throughout the tau protein will be important residues to target. The inventors' work with hairpin structures in tau indicates that there are “gatekeeper” sequences that mask aggregation-prone sequences in the protein. They predict that in disease states that the gatekeeper sequences will unmask the aggregation-prone sequences. This will allow epitope exposure on both gatekeeper sequences and aggregation-prone sequences that will be strong targets for diagnosis and therapy. Non-natural epitopes that exploit trans-proline residues at these sites will represent important therapeutic avenues.
3. Epitopes encompassing aa 130-160 will represent important targets to react with pathological forms of tau. XL-MS studies elucidated in Mirbaha et al. (2018) indicate that one region of tau undergoes dramatic conformational change when tau converts from an inert form (Mi) to a seed-competent form (Ms). This involves contacts between aa140-150 within the repeat domain. This indicates a dramatic conformational shift in the peptide. The inventor predicts that this will reveal new amino acids for interaction with therapeutic or diagnostic antibodies. Encompassing flanking peptides brings the range of potential peptide targets to 130-160.
4. Specific pSer262 is uniquely associated with pathological forms of tau monomer. The inventor predicts that epitopes that include this phospho-residue will be productive in discriminating pathological tau from normal forms in therapeutic and diagnostic applications. This is based on mass spectrometry analyses of pathological vs. normal tau monomer from mouse and human brain samples.

The following table provides exemplary tau peptide sequences according to the present disclosure:

TABLE B
TAU PEPTIDES

1) R2/R3 hairpin (295-311):
295-311
DNIKHVPGGGSVQIVYK (SEQ ID NO: 4)
295-311 P301L
DNIKHVLGGGSVQIVYK (SEQ ID NO: 5)
295-311 P301S
DNIKHVSGGGSVQIVYK (SEQ ID NO: 6)
295-311 (Fluorinated 45-F-Proline; cis)
DNIKHV[4S-FPro] GGGSVQIVYK (SEQ ID NO: 7)
295-311 (Fluorinated 4R-F-Proline; trans)
DNIKHV[4R-FPro] GGGSVQIVYK (SEQ ID NO: 8)
2) N-term fragment of R2/R3 hairpin (295-305):
295-305
DNIKHVPGGGS (SEQ ID NO: 9)
295-305 (P301L)
DNIKHVLGGGS (SEQ ID NO: 10)
295-305 (P301S)
DNIKHVSGGGS (SEQ ID NO: 11)
295-305 (Fluorinated 45-F-Proline; cis)
DNIKHV[4S-FPro] GGGS (SEQ ID NO: 12)
295-305 (Fluorinated 4R-F-Proline; trans)
DNIKHV[4R-FPro] GGGS (SEQ ID NO: 13)
3) C-term fragment of R2/R3 hairpin (300-311):
300-311
VPGGGSVQIVYK (SEQ ID NO: 14)
300-311 P301L
VLGGGSVQIVYK (SEQ ID NO: 15)
300-311 P301S
VSGGGSVQIVYK (SEQ ID NO: 16)
300-311 (Fluorinated 45-F-Proline; cis)
V[4S-FPro] GGGSVQIVYK (SEQ ID NO: 17)
300-311 (Fluorinated 4R-F-Proline; trans)
V[4R-FPro] GGGSVQIVYK (SEQ ID NO: 18)
4) R1/R2 hairpin (258- or 263-281):
263-281
TENLKHQPGGGKVQIINKK (SEQ ID NO: 19)
263-281 (P270L)
TENLKHQLGGGKVQIINKK (SEQ ID NO: 20)
263-281 (P270S)
TENLKHQSGGGKVQIINKK (SEQ ID NO: 21)
263-281 (Fluorinated 45-F-Proline; cis)
TENLKHQ[4S-FPro]GGGKVQIINKK (SEQ ID NO: 22)
263-281 (Fluorinated 4R-F-Proline; trans)
TENLKHQ[4R-FPro]GGGKVQIINKK (SEQ ID NO: 23)
258-281 (pS262)
SKIGpSTENLKHQPGGGKVQIINKK (SEQ ID NO: 24)
258-281 (P270L)
SKIGpSTENLKHQLGGGKVQIINKK (SEQ ID NO: 25)
258-281 (P270S)
SKIGpSTENLKHQSGGGKVQIINKK (SEQ ID NO: 26)
258-281 (Fluorinated 45-F-Proline cis)
SKIGpSTENLKHQ[4S-FPro]GGGKVQIINKK (SEQ ID NO: 27)
258-281 (Fluorinated 4R-F-Proline trans)
SKIGpSTENLKHQ[4R-FPro]GGGKVQIINKK (SEQ ID NO: 28)
5) N-term fragment of R1/R2 hairpin (258- or 263-274):
263-274
TENLKHQPGGGK (SEQ ID NO: 29)
263-274 (P270L)
TENLKHQLGGGK (SEQ ID NO: 30)
263-274 (P270S)
TENLKHQSGGGK (SEQ ID NO: 31)
263-274 (Fluorinated 45-F-Proline; cis)
TENLKHQ[4S-FPro]GGGK (SEQ ID NO: 32)
263-274 (Fluorinated 4R-F-Proline; trans)
TENLKHQ[4R-FPro]GGGK (SEQ ID NO: 33)
258-274 (pS262)
SKIGpSTENLKHQPGGGK (SEQ ID NO: 34)
258-274 (P270L)
SKIGpSTENLKHQLGGGK (SEQ ID NO: 35)
258-274 (P270S)
SKIGpSTENLKHQSGGGK (SEQ ID NO: 36)
258-274 (Fluorinated 45-F-Proline cis)
SKIGpSTENLKHQ[4S-FPro]GGGK (SEQ ID NO: 37)
258-274 (Fluorinated 4R-F-Proline trans)
SKIGpSTENLKHQ[4R-FPro]GGGK (SEQ ID NO: 38)
6) C-terminal fragment of R1/R2 hairpin (269-281):
269-281
QPGGGKVQIINKK (SEQ ID NO: 39)
269-281 (P270L)
QLGGGKVQIINKK (SEQ ID NO: 40)
269-281 (P270S)
QSGGGKVQIINKK (SEQ ID NO: 41)
269-281 (Fluorinated 45-F-Proline; cis)
Q[4S-FPro]GGGKVQIINKK (SEQ ID NO: 42)
269-281 (Fluorinated 4R-F-Proline; trans)
Q[4R-FPro]GGGKVQIINKK (SEQ ID NO: 43)
7) R3/R4 hairpin (326-343):
326-343
GNIHHKPGGGQVEVKSEK (SEQ ID NO: 44)
326-343 (P332L)
GNIHHKLGGGQVEVKSEK (SEQ ID NO: 45)
326-343 (P332S)
GNIHHKSGGGQVEVKSEK (SEQ ID NO: 46)
326-343 (Fluorinated 45-F-Proline; cis)
GNIHHK[4S-FPro]GGGQVEVKSEK (SEQ ID NO: 47)
326-343 (Fluorinated 4R-F-Proline; trans)
GNIHHK[4R-FPro]GGGQVEVKSEK (SEQ ID NO: 48)
8) N-terminal fragment of R3/R4 hairpin (326-336):
326-336
GNIHHKPGGGQ (SEQ ID NO: 49)
326-336(P332L)
GNIHHKLGGGQ (SEQ ID NO: 50)
326-336(P3325)
GNIHHKSGGGQ (SEQ ID NO: 51)
326-336 (Fluorinated 45-F-Proline; cis)
GNIHHK[4S-FPro]GGGQ (SEQ ID NO: 52)
326-336(Fluorinated 4R-F-Proline; trans)
GNIHHK[4R-FPro]GGGQ (SEQ ID NO: 53)
9) C-terminal fragment of R3/R4 hairpin (331-343):
331-343
KPGGGQVEVKSEK (SEQ ID NO: 54)
331-343 (P332L)
KLGGGQVEVKSEK (SEQ ID NO: 55)
331-343 (P332S)
KSGGGQVEVKSEK (SEQ ID NO: 56)
331-343 (Fluorinated 45-F-Proline; cis)
K[4S-FPro]GGGQVEVKSEK (SEQ ID NO: 57)
331-343 (Fluorinated 4R-F-Proline; trans)
K[4R-FPro]GGGQVEVKSEK (SEQ ID NO: 58)
10) R4 hairpin 358-375:
358-375
DNITHVPGGGNKKIETHK (SEQ ID NO: 59)
358-375 (P364L)
DNITHVLGGGNKKIETHK (SEQ ID NO: 60)
358-375 (P364S)
DNITHVSGGGNKKIETHK (SEQ ID NO: 61)
358-375 (Fluorinated 45-F-Proline; cis)
DNITHV[4S-FPro]GGGNKKIETHK (SEQ ID NO: 62)
358-375 (Fluorinated 4R-F-Proline; trans)
DNITHV[4R-FPro]GGGNKKIETHK (SEQ ID NO: 63)
11) N-terminal fragment of R4 hairpin (358-368):
358-368
DNITHVPGGGN (SEQ ID NO: 64)
358-368 (P364L)
DNITHVLGGGN (SEQ ID NO: 65)
358-368 (P364S)
DNITHVSGGGN (SEQ ID NO: 66)
358-368 (Fluorinated 45-F-Proline; cis)
DNITHV[4S-FPro]GGGN (SEQ ID NO: 67)
358-368 (Fluorinated 4R-F-Proline; trans)
DNITHV[4R-FPro]GGGN (SEQ ID NO: 68)
12) C-terminal fragment of R4 hairpin (363-375):
363-375
VPGGGNKKIETHK (SEQ ID NO: 69)
363-375 (P364L)
VLGGGNKKIETHK (SEQ ID NO: 70)
363-375 (P364S)
VSGGGNKKIETHK (SEQ ID NO: 71)
363-375 (Fluorinated 45-F-Proline; cis)
V[4S-FPro]GGGNKKIETHK (SEQ ID NO: 72)
363-375 (Fluorinated 4R-F-Proline; trans)
V[4R-FPro]GGGNKKIETHK (SEQ ID NO: 73)
13) Intermediate fragments between hairpins
231-242 TPPKSPSSAKSR (SEQ ID NO: 150)
237-255 SSAKSRLQTAPVPMPDLKN (SEQ ID NO: 151)
243-262 LQTAPVPMPDLKNVKSKIGS (SEQ ID NO: 74)
253-262 LKNVKSKIGS (SEQ ID NO: 75)
243-252 LQTAPVPMPD (SEQ ID NO: 76)
281-294 KLDLSNVQSKCGSK (SEQ ID NO: 77)
288-294 QSKCGSK (SEQ ID NO: 78)
281-287 KLDLSNV (SEQ ID NO: 79)
312-325 PVDLSKVTSKCGSL (SEQ ID NO: 80)
319-325 TSKCGSL (SEQ ID NO: 81)
312-318 PVDLSKV (SEQ ID NO: 82)
344-357 LDFKDRVQSKIGSL (SEQ ID NO: 83)
351-357 QSKIGSL (SEQ ID NO: 84)
344-350 LDFKDRV (SEQ ID NO: 85)
376-395 LTFRENAKAKTDHGAEIVYK (SEQ ID NO: 86)
385-395 KTDHGAEIVYK (SEQ ID NO: 87)
376-384 LTFRENAKA (SEQ ID NO: 88)
14) Fragments within Tau274-311 antigen:
274-288
KVQIINKKLDLSNVQ (SEQ ID NO: 89)
289-300
SKCGSKDNIKHV (SEQ ID NO: 90)
305-320
SVQIVYKPVDLSKVTS (SEQ ID NO: 91) (extends past original
antigen but keeps the post-VQIVYK sequence symmetric)
15) Truncations of Tau274-311 from both N-term and C-term ends:
C-term truncations
KVQIINK (SEQ ID NO: 92)
KVQIINKKLDLSNVQSKC (SEQ ID NO: 93)
KVQIINKKLDLSNVQSKCGSK (SEQ ID NO: 94)
KVQIINKKLDLSNVQSKCGSKDNIKHV (SEQ ID NO: 95)
KVQIINKKLDLSNVQSKCGSKDNIKHVPGGGS (SEQ ID NO: 96)
N-term truncations
KLDLSNVQSKCGSKDNIKHVPGGGSVOIVYK (SEQ ID NO: 97)
CGSKDNIKHVPGGGSVQIVYK (SEQ ID NO: 98)
PGGGSVQIVYK (SEQ ID NO: 99)
Conformational variants from N-term truncations
P301L
KLDLSNVQSKCGSKDNIKHVLGGGSVOIVY (SEQ ID NO: 100)
P301L
CGSKDNIKHVLGGGSVQIVYK (SEQ ID NO: 101)
P301S
KLDLSNVQSKCGSKDNIKHVSGGGSVQIVYK (SEQ ID NO: 102)
P301S
CGSKDNIKHVSGGGSVQIVYK (SEQ ID NO: 103)
(Fluorinated 45-F-Proline; cis)
KLDLSNVQSKCGSKDNIKHV [4S-FPro] GGGSVQIVYK (SEQ ID NO: 104)
(Fluorinated 4R-F-Proline; trans)
KLDLSNVQSKCGSKDNIKHV [4R-FPro] GGGSVQIVYK (SEQ ID NO: 105)
16) Conformational mutants of Tau274-311 antigen
274-311 (P301L)
KVQIINKKLDLSNVQSKCGSKDNIKHV LGGGSVQIVY K (SEQ ID NO: 106)
274-311 (P301S)
KVQIINK KLDLSNVQSK CGSKDNIKHV SGGGSVQIVY K (SEQ ID NO: 107)
274-311 (Fluorinated 4S-F-Proline; cis)
KVQIINK KLDLSNVQSK CGSKDNIKHV [4S-FPro] GGGSVOIVY K
(SEQ ID NO: 108)
274-311 (Fluorinated 4R-F-Proline; trans)
KVQIINK KLDLSNVQSK CGSKDNIKHV [4R-FPro] GGGSVOIVY K
(SEQ ID NO: 109)
17) 3R
aa263-311: TENLKHQP GGGKVQIVY K (SEQ ID NO: 110)
18) 4R
aa18-34: YGL GDRKDQGGYT MHQD (SEQ ID NO: 111)
aa22-34: DRKDQGGYT MHQD (SEQ ID NO: 112)
aa22-38: RKDQGGYT MHQDQEGD (SEQ ID NO: 113)
aa131-141: SKDGTGSDDK K (SEQ ID NO: 114)
aa130-160: KSKDGTGSDDKKAKGADGKTK IATPRGAAPP (SEQ ID NO: 115)
aa263-281: TENLKHQP GGGKVQIINK K (SEQ ID NO: 116)
aa275-293: VQIINK KLDLSNVQSK CGS (SEQ ID NO: 117)
aa294-311: KDNIKHV PGGGSVQIVY K (SEQ ID NO: 118)
aa326-343: GNIHH KPGGGQVEVK SEK (SEQ ID NO: 119)
aa358-375: DNI THVPGGGNKK IETHK (SEQ ID NO: 120)
19) R1/R3 hairpin (263-280)
263-281
TENLKHQPGGGKVQIVYK (SEQ ID NO: 121)
P270L 263-280
TENLKHQLGGGKVQIVYK (SEQ ID NO: 122)
P270S 263-2810
TENLKHQSGGGKVQIVYK (SEQ ID NO: 123)
263-280 (Fluorinated 45-F-Proline cis)
TENLKHQ[4S-F-Pro cis]GGGKVQIVYK (SEQ ID NO: 124)
263-280 (Fluorinated 4R-F-Proline trans)
TENLKHQ[4R-F-Pro trans]GGGKVQIVYK (SEQ ID NO: 125)
258-280
SKIGpSTENLKHQPGGGKVQIVYK (SEQ ID NO: 126)
P270L 258-280
SKIGpSTENLKHQLGGGKVQIVYK (SEQ ID NO: 127)
P270S 258-280
SKIGpSTENLKHQSGGGKVQIVYK (SEQ ID NO: 128)
258-280 (Fluorinated 45-F-Proline cis)
SKIGpSTENLKHQ[4S-F-Pro cis]GGGKVQIVYK (SEQ ID NO: 129)
258-280 (Fluorinated 4R-F-Proline trans)
SKIGpSTENLKHQ[4R-F-Pro trans]GGGKVQIVYK (SEQ ID NO: 130)
20) N-term fragment of R1/R3 hairpin (263-274)
263-274
TENLKHQPGGGK (SEQ ID NO: 131)
P270L 263-274
TENLKHQLGGGK (SEQ ID NO: 132)
P270S 263-274
TENLKHQSGGGK (SEQ ID NO: 133)
263-274 (Fluorinated 45-F-Proline cis)
TENLKHQ[4S-F-Pro cis]GGGK (SEQ ID NO: 134)
263-274 (Fluorinated 4R-F-Proline trans)
TENLKHQ[4R-F-Pro trans]GGGK (SEQ ID NO: 135)
258-274
SKIGpSTENLKHQPGGGK (SEQ ID NO: 136)
P270L 258-274
SKIGpSTENLKHQLGGGK (SEQ ID NO: 137)
P270S 258-274
SKIGpSTENLKHQSGGGK (SEQ ID NO: 138)
258-274 (Fluorinated 45-F-Proline cis)
SKIGpSTENLKHQ[4S-F-Pro cis]GGGK (SEQ ID NO: 139)
258-274 (Fluorinated 4R-F-Proline trans)
SKIGpSTENLKHQ[4R-F-Pro trans]GGGK (SEQ ID NO: 140)
21) C-term fragment of R1/R3 hairpin (269-280)
269-280
QPGGGKVQIVYK (SEQ ID NO: 141)
P270L 269-280
QLGGGKVQIVYK (SEQ ID NO: 142)
P270S 269-280
QSGGGKVQIVYK (SEQ ID NO: 143)
269-280 (Fluorinated 45-F-Proline cis)
Q[4S-F-Pro cis]GGGKVQIVYK (SEQ ID NO: 144)
269-280 (Fluorinated 4R-F-Proline trans)
Q[4R-F-Pro trans]GGGKVQIVYK (SEQ ID NO: 145)

### IV. ACTIVE/PASSIVE IMMUNIZATION AND TREATMENT/PREVENTION OF TAUOPATHIES

A. Formulation and Administration

The present disclosure provides pharmaceutical compositions comprising anti-tau antibodies and antigens for generating the same. Such compositions comprise a prophylactically or therapeutically effective amount of an antibody or a fragment thereof, or a peptide immunogen, and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a particular carrier when the pharmaceutical composition is administered intravenously. Saline solutions and aqueous dextrose and glycerol solutions can also be employed as liquid carriers, particularly for injectable solutions. Other suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulations can include standard carriers such as pharmaceutical grades of mannitol, lactose, starch, magnesium stearate, sodium saccharine, cellulose, magnesium carbonate, etc. Examples of suitable pharmaceutical agents are described in “Remington's Pharmaceutical Sciences.” Such compositions will contain a prophylactically or therapeutically effective amount of the antibody or fragment thereof, preferably in purified form, together with a suitable amount of carrier so as to provide the form for proper administration to the patient. The formulation should suit the mode of administration, which can be oral, intravenous, intraarterial, intrabuccal, intranasal, nebulized, bronchial inhalation, intra-rectal, vaginal, topical or delivered by mechanical ventilation.

Active vaccines are also envisioned where antibodies like those disclosed are produced in vivo in a subject at risk of developing a tauopathy. Such vaccines can be formulated for parenteral administration, e.g., formulated for injection via the intradermal, intravenous, intramuscular, subcutaneous, or even intraperitoneal routes Administration by intradermal and intramuscular routes are contemplated. The vaccine could alternatively be administered by a topical route directly to the mucosa, for example by nasal drops, inhalation, by nebulizer, or via intrarectal or vaginal delivery. Pharmaceutically acceptable salts include the acid salts and those which are formed with inorganic acids such as, for example, hydrochloric or phosphoric acids, or such organic acids as acetic, oxalic, tartaric, mandelic, and the like. Salts formed with the free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Passive transfer of antibodies, known as artificially acquired passive immunity, generally will involve the use of intravenous or intramuscular injections. The forms of antibody can be human or animal blood plasma or serum, as pooled human immunoglobulin for intravenous (IVIG) or intramuscular (IG) use, as high-titer human IVIG or IG from immunized or from donors recovering from disease, and as monoclonal antibodies (MAb). Such immunity generally lasts for only a short period of time, and there is also a potential risk for hypersensitivity reactions, and serum sickness, especially from gamma globulin of non-human origin. However, passive immunity provides immediate protection. The antibodies will be formulated in a carrier suitable for injection, i.e., sterile and syringeable.

Generally, the ingredients of compositions of the disclosure are supplied either separately or mixed together in unit dosage form, for example, as a dry lyophilized powder or water-free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

2. ADCC

Antibody-dependent cell-mediated cytotoxicity (ADCC) is an immune mechanism leading to the lysis of antibody-coated target cells by immune effector cells. The target cells are cells to which antibodies or fragments thereof comprising an Fc region specifically bind, generally via the protein part that is N-terminal to the Fc region. By “antibody having increased/reduced antibody dependent cell-mediated cytotoxicity (ADCC)” is meant an antibody having increased/reduced ADCC as determined by any suitable method known to those of ordinary skill in the art.

As used herein, the term “increased/reduced ADCC” is defined as either an increase/reduction in the number of target cells that are lysed in a given time, at a given concentration of antibody in the medium surrounding the target cells, by the mechanism of ADCC defined above, and/or a reduction/increase in the concentration of antibody, in the medium surrounding the target cells, required to achieve the lysis of a given number of target cells in a given time, by the mechanism of ADCC. The increase/reduction in ADCC is relative to the ADCC mediated by the same antibody produced by the same type of host cells, using the same standard production, purification, formulation and storage methods (which are known to those skilled in the art), but that has not been engineered. For example, the increase in ADCC mediated by an antibody produced by host cells engineered to have an altered pattern of glycosylation (e.g., to express the glycosyltransferase, GnTIII, or other glycosyltransferases) by the methods described herein, is relative to the ADCC mediated by the same antibody produced by the same type of non-engineered host cells.

3. CDC

Complement-dependent cytotoxicity (CDC) is a function of the complement system. It is the processes in the immune system that kill pathogens by damaging their membranes without the involvement of antibodies or cells of the immune system. There are three main processes. All three insert one or more membrane attack complexes (MAC) into the pathogen which cause lethal colloid-osmotic swelling, i.e., CDC.

### V. ANTIBODY CONJUGATES

Antibodies of the present disclosure may be linked to at least one agent to form an antibody conjugate. In order to increase the efficacy of antibody molecules as diagnostic or therapeutic agents, it is conventional to link or covalently bind or complex at least one desired molecule or moiety. Such a molecule or moiety may be, but is not limited to, at least one effector or reporter molecule. Effector molecules comprise molecules having a desired activity, e.g., cytotoxic activity. By contrast, a reporter molecule is defined as any moiety which may be detected using an assay. Non-limiting examples of reporter molecules which have been conjugated to antibodies include enzymes, radiolabels, haptens, fluorescent labels, phosphorescent molecules, chemiluminescent molecules, chromophores, photoaffinity molecules, colored particles or ligands, such as biotin.

Antibody conjugates are generally preferred for use as diagnostic agents. Antibody diagnostics generally fall within two classes, those for use in in vitro diagnostics, such as in a variety of immunoassays, and those for use in vivo diagnostic protocols, generally known as “antibody-directed imaging.” Many appropriate imaging agents are known in the art, as are methods for their attachment to antibodies (see, for e.g., U.S. Pat. Nos. 5,021,236, 4,938,948, and 4,472,509). The imaging moieties used can be paramagnetic ions, radioactive isotopes, fluorochromes, NMR-detectable substances, and X-ray imaging agents.

In the case of paramagnetic ions, one might mention by way of example ions such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and/or erbium (III), with gadolinium being particularly preferred. Ions useful in other contexts, such as X-ray imaging, include but are not limited to lanthanum (III), gold (III), lead (II), and especially bismuth (III).

In the case of radioactive isotopes for therapeutic and/or diagnostic application, one might mention astatine²¹¹, ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine^{123,} iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technicium^{99m} and/or yttrium⁹⁰. ¹²⁵I is often being preferred for use in certain embodiments, and technicium^{99m} and/or indium¹¹¹ are also often preferred due to their low energy and suitability for long range detection. Radioactively labeled monoclonal antibodies of the present disclosure may be produced according to well-known methods in the art. For instance, monoclonal antibodies can be iodinated by contact with sodium and/or potassium iodide and a chemical oxidizing agent such as sodium hypochlorite, or an enzymatic oxidizing agent, such as lactoperoxidase. Monoclonal antibodies according to the disclosure may be labeled with technetium^{99m} by ligand exchange process, for example, by reducing pertechnate with stannous solution, chelating the reduced technetium onto a Sephadex column and applying the antibody to this column. Alternatively, direct labeling techniques may be used, e.g., by incubating pertechnate, a reducing agent such as SNCl₂, a buffer solution such as sodium-potassium phthalate solution, and the antibody. Intermediary functional groups which are often used to bind radioisotopes which exist as metallic ions to antibody are diethylenetriaminepentaacetic acid (DTPA) or ethylene diaminetetracetic acid (EDTA).

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red.

Additional types of antibodies contemplated in the present disclosure are those intended primarily for use in vitro, where the antibody is linked to a secondary binding ligand and/or to an enzyme (an enzyme tag) that will generate a colored product upon contact with a chromogenic substrate. Examples of suitable enzymes include urease, alkaline phosphatase, (horseradish) hydrogen peroxidase or glucose oxidase. Preferred secondary binding ligands are biotin and avidin and streptavidin compounds. The use of such labels is well known to those of skill in the art and are described, for example, in U.S. Pat. Nos. 3,817,837, 3,850,752, 3,939,350, 3,996,345, 4,277,437, 4,275,149 and 4,366,241.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Molecules containing azido groups may also be used to form covalent bonds to proteins through reactive nitrene intermediates that are generated by low intensity ultraviolet light (Potter and Haley, 1983). In particular, 2- and 8-azido analogues of purine nucleotides have been used as site-directed photoprobes to identify nucleotide binding proteins in crude cell extracts (Owens & Haley, 1987; Atherton et al., 1985). The 2- and 8-azido nucleotides have also been used to map nucleotide binding domains of purified proteins (Khatoon et al., 1989; King et al., 1989; Dholakia et al., 1989) and may be used as antibody binding agents.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3α-6α-diphenylglycouril-3 attached to the antibody (U.S. Pat. Nos. 4,472,509 and 4,938,948). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

In other embodiments, derivatization of immunoglobulins by selectively introducing sulfhydryl groups in the Fc region of an immunoglobulin, using reaction conditions that do not alter the antibody combining site are contemplated. Antibody conjugates produced according to this methodology are disclosed to exhibit improved longevity, specificity and sensitivity (U.S. Pat. No. 5,196,066, incorporated herein by reference). Site-specific attachment of effector or reporter molecules, wherein the reporter or effector molecule is conjugated to a carbohydrate residue in the Fc region have also been disclosed in the literature (O'Shannessy et al., 1987). This approach has been reported to produce diagnostically and therapeutically promising antibodies which are currently in clinical evaluation.

### VI. IMMUNODETECTION/DIAGNOSTIC METHODS

In still further embodiments, the present disclosure concerns immunodetection methods for binding, purifying, removing, quantifying and otherwise generally detecting tau protein and its associated antigens. While such methods can be applied in a traditional sense, another use will be in quality control and monitoring of vaccine and other virus stocks, where antibodies according to the present disclosure can be used to assess the amount or integrity (i.e., long term stability) of antigens in viruses. Alternatively, the methods may be used to screen various antibodies for appropriate/desired reactivity profiles.

Other immunodetection methods include specific assays for determining the presence of tau protein in a subject, such as for the purpose of diagnosing or differentiating tauopathies. A wide variety of assay formats are contemplated, but specifically those that would be used to detect tau protein in a fluid obtained from a subject, such as saliva, blood, plasma, sputum, semen or urine. The assays may be advantageously formatted for non-healthcare (home) use, including lateral flow assays (see below) analogous to home pregnancy tests. These assays may be packaged in the form of a kit with appropriate reagents and instructions to permit use by the subject of a family member.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. In particular, a competitive assay for the detection and quantitation of tau antibodies directed to specific parasite epitopes in samples also is provided. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample suspected of containing tau protein, and contacting the sample with a first antibody in accordance with the present disclosure, as the case may be, under conditions effective to allow the formation of immunocomplexes.

These methods include methods for purifying tau protein or related antigens from a sample. The antibody will preferably be linked to a solid support, such as in the form of a column matrix, and the sample suspected of containing the tau protein or antigenic component will be applied to the immobilized antibody. The unwanted components will be washed from the column, leaving the tau antigen immunocomplexed to the immobilized antibody, which is then collected by removing the organism or antigen from the column.

The immunobinding methods also include methods for detecting and quantifying the amount of tau protein or related components in a sample and the detection and quantification of any immune complexes formed during the binding process. Here, one would obtain a sample suspected of containing tau protein or its related antigens and contact the sample with an antibody that binds tau protein or components thereof, followed by detecting and quantifying the amount of immune complexes formed under the specific conditions. In terms of antigen detection, the biological sample analyzed may be any sample that is suspected of containing tau protein or related antigens, such as a tissue section or specimen, a homogenized tissue extract, a biological fluid, including blood and serum, or a secretion, such as feces or urine.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to tau protein or related antigens present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

A. ELISAs

Immunoassays, in their most simple and direct sense, are binding assays. Certain preferred immunoassays are the various types of enzyme linked immunosorbent assays (ELISAs) and radioimmunoassays (RIA) known in the art Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the disclosure are immobilized onto a selected surface exhibiting protein affinity, such as a well in a polystyrene microtiter plate. Then, a test composition suspected of containing the tau protein or related antigen is added to the wells. After binding and washing to remove non-specifically bound immune complexes, the bound antigen may be detected. Detection may be achieved by the addition of another anti-tau antibody that is linked to a detectable label. This type of ELISA is a simple “sandwich ELISA.” Detection may also be achieved by the addition of a second anti-tau antibody, followed by the addition of a third antibody that has binding affinity for the second antibody, with the third antibody being linked to a detectable label.

In another exemplary ELISA, the samples suspected of containing the tau antigen are immobilized onto the well surface and then contacted with the anti-tau antibodies of the disclosure. After binding and washing to remove non-specifically bound immune complexes, the bound anti-tau antibodies are detected. Where the initial anti-tau antibodies are linked to a detectable label, the immune complexes may be detected directly. Again, the immune complexes may be detected using a second antibody that has binding affinity for the first anti-tau antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C., or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

In another embodiment, the present disclosure contemplates the use of competitive formats. This is particularly useful in the detection of tau antibodies in sample. In competition-based assays, an unknown amount of analyte or antibody is determined by its ability to displace a known amount of labeled antibody or analyte. Thus, the quantifiable loss of a signal is an indication of the amount of unknown antibody or analyte in a sample.

B. Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. However, it should be noted that bacteria, virus or environmental samples can be the source of protein and thus Western blotting is not restricted to cellular studies only. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF but are far more fragile and do not stand up well to repeated probings. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

C. Lateral Flow Assays

Lateral flow assays, also known as lateral flow immunochromatographic assays, are simple devices intended to detect the presence (or absence) of a target analyte in sample (matrix) without the need for specialized and costly equipment, though many laboratory-based applications exist that are supported by reading equipment. Typically, these tests are used as low resources medical diagnostics, either for home testing, point of care testing, or laboratory use. A widely spread and well-known application is the home pregnancy test.

The technology is based on a series of capillary beds, such as pieces of porous paper or sintered polymer. Each of these elements has the capacity to transport fluid (e.g., urine) spontaneously. The first element (the sample pad) acts as a sponge and holds an excess of sample fluid. Once soaked, the fluid migrates to the second element (conjugate pad) in which the manufacturer has stored the so-called conjugate, a dried format of bio-active particles (see below) in a salt-sugar matrix that contains everything to guarantee an optimized chemical reaction between the target molecule (e.g., an antigen) and its chemical partner (e.g., antibody) that has been immobilized on the particle's surface. While the sample fluid dissolves the salt-sugar matrix, it also dissolves the particles and in one combined transport action the sample and conjugate mix while flowing through the porous structure. In this way, the analyte binds to the particles while migrating further through the third capillary bed. This material has one or more areas (often called stripes) where a third molecule has been immobilized by the manufacturer. By the time the sample-conjugate mix reaches these strips, analyte has been bound on the particle and the third ‘capture’ molecule binds the complex. After a while, when more and more fluid has passed the stripes, particles accumulate and the stripe-area changes color. Typically, there are at least two stripes: one (the control) that captures any particle and thereby shows that reaction conditions and technology worked fine, the second contains a specific capture molecule and only captures those particles onto which an analyte molecule has been immobilized. After passing these reaction zones, the fluid enters the final porous material—the wick—that simply acts as a waste container. Lateral Flow Tests can operate as either competitive or sandwich assays. Lateral flow assays are disclosed in U.S. Pat. No. 6,485,982.

D. Immunohistochemistry

The antibodies of the present disclosure may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

E. Immunodetection Kits

In still further embodiments, the present disclosure concerns immunodetection kits for use with the immunodetection methods described above. As the antibodies may be used to detect tau antigens, the antibodies may be included in the kit. The immunodetection kits will thus comprise, in suitable container means, a first antibody that binds tau antigen, and optionally an immunodetection reagent.

In certain embodiments, the anti-tau antibody may be pre-bound to a solid support, such as a column matrix and/or well of a microtiter plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first antibody.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with the present disclosure.

The kits may further comprise a suitably aliquoted composition of tau or related antigens, such as a peptide comprising an epitope described herein, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits of the present disclosure will also typically include a means for containing the antibody, antigen, and any other reagent containers in close confinement for commercial sale. Such containers may include injection or blow-molded plastic containers into which the desired vials are retained.

### VII. EXAMPLES

The following examples are included to demonstrate particular embodiments of the disclosure. It should be appreciated by those of skill in the art that the techniques disclosed in the examples which follow represent techniques discovered by the inventor to function well in the practice of the disclosure, and thus can be considered to constitute particular modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Materials and Methods

Peptides were designed according to the inventor's knowledge of the formation of inhibitory hairpins at the interfaces of R1/R2, R2/R3, R3/R4 and R4 terminus that center on the PGGG motifs. The design strategy centers on the idea that the the hairpin conformation changes between soluble and seed competent conformations observed within the repeat domain. The initial strategy captures epitopes surrounding the hairpins in both WT and mutant contexts but also incorporating proline analogs ([4S-FPro] and [4R-FPro]) that promote conversion between cis and trans conformations; cis being protective from aggregation and trans being pro-aggregation. In addition to probing intact hairpins, the inventor engineered epitopes that contain the left and right side of the hairpin—perhaps in these instances it will not be necessary to control the conformation. Certain antigens (14-16) include simple perturbations of the 275-311 epitope.

### Example 2

### Results and Discussion

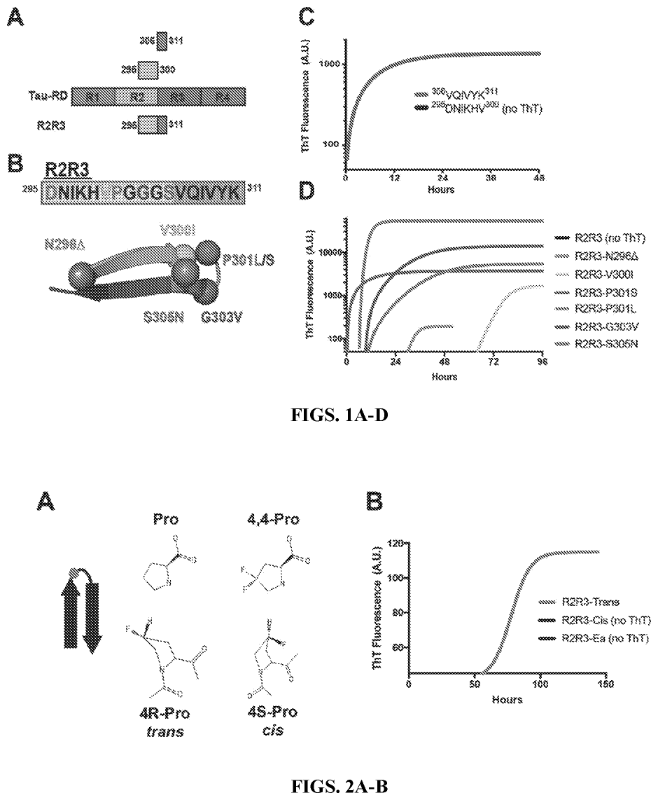

Tau amyloid formation is governed by flanking residues. In the tau protein, the ³⁰⁶VQIVYK³¹¹ (SEQ ID NO: 1) sequence is a key motif that drives amyloid formation. It is encoded within the tau repeat domain at the start of R3 (FIG. 1A). In solution, ³⁰⁶VQIVYK³¹¹ (SEQ ID NO: 1) hexapeptides aggregate spontaneously and rapidly as measured by Thioflavin T (ThT) fluorescence intensity whereas the N-terminal sequence ²⁹⁵DNIKHV³⁰⁰ (SEQ ID NO: 2) does not aggregate (FIG. 1B). Combining these sequences yields a minimal structural element spanning R2 into R3 (²⁹⁵DNIKHVPGGGSVQIVYK³¹¹ (SEQ ID NO: 4), R2R3) predicted by in silico modelling (FIGS. 1A-C). This sequence is also where numerous disease-associated mutations cluster. Indeed, WT peptide fragments representing this motif do not aggregate readily, with no ThT detected up to 96 hrs (FIG. 1D). By contrast, disease-associated mutations (FIG. 1D) substituted into the R2R3 peptide fragment were sufficient to generate spontaneous amyloid formation: R2R3-P301S, R2R3-P301L, R2R3-N296Δ, R2R3-G303V, R2R3-S305N, and R2R3-V300I. Each of these peptides was confirmed to form amyloid-like fibril morphologies by transmission electron microscopy, except for the wild-type R2R3 peptide where no large structures were found.

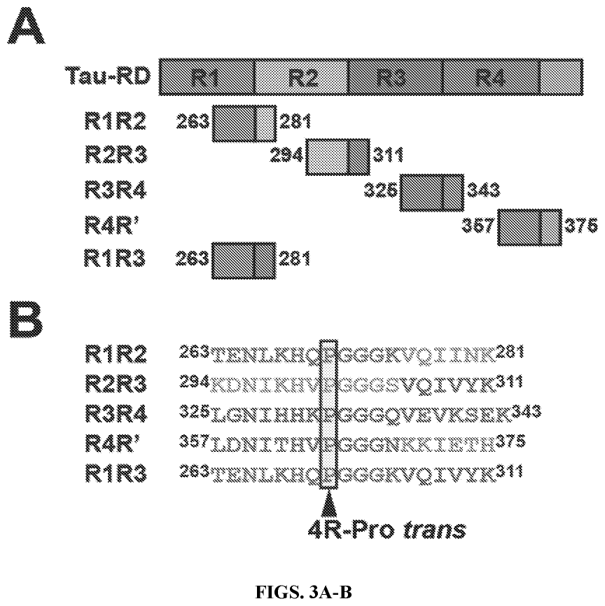

Proline 301 cis-trans isomerization modulates aggregation. Proline isomerization events in tau have been proposed to play a role in aggregation and disease, but P301 isomerization has not been linked to tau aggregation and pathology. Serine or leucine substitutions at P301 proximal to ³⁰⁶VQIVYK³¹¹ (SEQ ID NO: 1) drastically alter aggregation propensity. The inventor hypothesized that P301 plays a crucial role inducing a β-turn in a P-G-G-G motif, which mediates a collapsed structure. They tested whether isomerization of P301 could influence spontaneous amyloid formation. They constructed a series of R2R3 peptides with proline analogs that preferentially populate either: (1) a cis rotamer (2S,4S)-fluoroproline; (2) a trans rotamer (2S,4R)-fluoroproline; or (3) a proline analog that easily interconverts between cis and trans (4,4)-difluoroproline (FIG. 2A). Only R2R3-Trans spontaneously aggregated (FIG. 2B), indicating the potential for proline isomerization events in tau pathogenesis. Additionally, the inventor anticipates that conformational changes in other regions within the repeat domain of tau including the R1R2, R3R4, R4R′ fragments and the splicing variant R1R3 may provide suitable sites to target tau pathogenesis or detect pathological forms (FIGS. 3A-B).

Summary of important hairpin regions. Prior work indicates the R2R3 domain encompasses a critical hairpin (detailed in FIGS. 1A-2B). Based on homologies and predicted structures, the inventor anticipates that other junctional domains will also embody hairpin structures that will undergo conformational changes in different tauopathies. Thus, the work on R2R3 has informed an understanding of the entire RD region of tau.

### Example 3

### Materials and Methods

Antibodies. Linear peptide antigens were synthesized by a contract research organization (Genscript, Piscataway, N.J.) and conjugated to KLH via an added N-terminal cysteine residue. For synthetic trans-proline residues Fmoc-trans-4-Fluoro-L-Proline was used and for synthetic cis-proline residues Fmoc-cis-4-Fluoro-L-Proline was used. The linear peptide epitopes were as follows:

a.a. 22-34-
DRKDQGGYTMHQD
a.a. 145-160-
ADGKTKIATPRGAAPP
a.a. 244-266-
QTAPVPMPDLKNVKSKIGSTENL
a.a. 259-266-
KIGSTENL
a.a. 263-281 (R1R2) trans-P270-
TENLKHQ[trans-P]GGGKVQIINKK
a.a. 263-311 Δ275-305 (R1R3) trans-P270-
TENLKHQ[trans-P]GGGKQIVYK
a.a. 294-311 (R2R3) trans-P301-
KDNIKHV[trans-P]GGGVQIVYK
a.a. 294-311 (R2R3) cis-P301-
KDNIKHV[cis-P]GGGSVQIVYK
a.a. 326-343 (R3R4) trans-P332-
GNIHHK[trans-P]GGGQVEVKSEK
a.a. 358-375 (R4R') trans-P364-
DNITHV[trans-P]GGGNKKIETHK

Mice or rabbits were inoculated with KLH-conjugated peptide antigens for production of monoclonal or polyclonal antibodies respectively. For mouse monoclonal antibodies, multiple hybridomas were screened by using conditioned media from the hybridomas for immunoprecipitation (IP). Rabbit polyclonal antibodies were affinity-purified prior to use for IP.

Immunoprecipitations. Fifty microliters of Dynabeads protein-A (Thermo Fisher, Waltham, Mass.) were incubated with 100 ul of hybridoma-conditioned media or 10 ug affinity purified rabbit polyclonal antibody for one hour, washed twice with PBS-T (1× PBS with 0.02% Tween-20) and incubated with 15 ug total brain homogenate at 4° C. overnight. Supernatant was collected and Immunoprecipitate was eluted using low pH elution buffer (Thermo Fisher, Waltham, Mass.).

Seeding assay. For each IP, the seeding activity was measured in the eluent and the supernatant using HEK293 FRET biosensor cells expressing tauRD-CFP and tau-RD-YFP as previously described (Holmes et al., 2014). Each IP or supernatant sample was used to treat 3 wells of a 96-well plate. For each triplicate, 4.5 μl of Lipofectamine 2000 (Thermo Fisher, Waltham, Mass.) was suspended in 25.5 μl of OptiMEM and incubated at room temperature for 5 minutes. The Lipofectamine mix was then added to each sample and the mixture was incubated at room temperature for 30 minutes before adding to 3 wells of biosensor cells in a 96-well plate. After 48 hours, cells were fixed in 2% PFA and FRET signal was measured using flow cytometry to determine percent FRET positivity. For each sample of IP or supernatant, results were normalized to the seeding activity in a reference sample of 15 μl of brain homogenate (pre-IP quantity of homogenate). Normalized seeding activity of the IP versus the supernatant fraction was used to determine the IP efficiency of each antibody-brain pair.

### Example 4

### Results

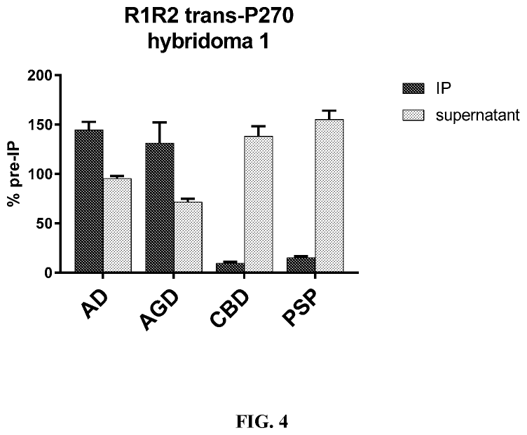

Comparison of the seeding activity in the IP and supernatant fractions of immunoprecipitations using different antibodies shows a variety of IP efficiency patterns. Homogenates of tissue from four human brains with histopathological diagnoses of Alzheimer's disease (AD), Argyrophilic Grain Disease (AGD), Progressive Supranuclear Palsy (PSP), and Corticobasal Degeneration (CBD) were used to screen the antibodies. FIG. 4 shows an example of the seeding activity data used to determine the IP efficiency for one of these antibodies, R1R2 hybridoma 1. This antibody immunoprecipitates pathological tau seeds from AD and AGD brain homogenates with a high efficiency as evidenced by the high level of seeding activity in the IP fraction and the comparatively low level of seeding activity remaining in the supernatant, but has a low IP efficiency for tau seeds from PSP and CBD brain. Table C summarizes the relative IP efficiencies of antibodies raised against 13 different linear peptide tau epitopes. Antibodies highlighted in green are affinity-purified rabbit polyclonal antibodies while those highlighted in red are unpurified mouse monoclonal antibodies. Several antibodies demonstrate disease-dependent IP efficiencies highlighted in yellow. The disease-distinguishing properties of antibodies targeting different epitopes reflect the structural differences between pathological tau species that define different tauopathies. Such antibodies are valuable basic-science research tools and have potential diagnostic utility. Further, they illustrate the importance of epitope selection in immunotherapy for tauopathies.

TABLE C
Performance of antibodies against
specific tau linear peptide epitopes
in immunoprecipitations of tau seeds
from human tauopathy brain homogenates

| Antibody | Sequence | AD | AGD | PSP | CBD |
|---|---|---|---|---|---|
| aa 22-34a | DRKDQGGYTMHQD | ++ | ++ | ++ | ++ |
| aa 145-160^{a} | ADGKTKIATPRGAAPP | + | + | + | + |
| TauA^{b} | QTAPVPMPDLKNVKSK | +++ | +++ | +++ | +++ |
| | IGSTENL | | | | |
| TauB^{b} | KIGSTENL | +++ | +++ | +++ | +++ |
| R1R2 | TENLKHQ[trans-P] | +++^{c} | +++^{c} | +^{c} | +^{c} |
| hybridoma 1^{a} | GGGKVQIINKK | | | | |
| R1R2 | TENLKHQ[trans-P] | +++ | +++ | ++^{c} | +^{c} |
| hybridoma 2^{a} | GGGKVQIINKK | | | | |
| R1R3 | TENLKHQ[trans-P] | +++^{c} | +++^{c} | −^{c} | −^{c} |
| hybridoma 1^{a} | GGGKVQIVYK | | | | |
| R1R3 | TENLKHQ[trans-P] | ++^{c} | +^{c} | −^{c} | −^{c} |
| hybridoma 2^{a} | GGGKVQIVYK | | | | |
| R2R3tP^{b} | KDNIKHV[trans-P] | ++ | ++ | ++ | ++ |
| | GGGSVQIVYK | | | | |
| R2R3cP^{b} | KDNIKHV[cis-P] | ++ | ++ | ++ | ++ |
| | GGGSVQIVYK | | | | |
| R3R4^{a} | GNIHHK[trans-P] | ++ | n/a | + | + |
| | GGGQVEVKSEK | | | | |
| R4R' | DNITHV[trans-P] | ++ | n/a | +^{c} | ++^{c} |
| hybridoma 1^{a} | GGGNKKIETHK | | | | |
| R4R' | DNITHV[trans-P] | + | n/a | − | + |
| hybridoma 2^{a} | GGGNKKIETHK | | | | |

Key
^{a}Custom Mouse mAb
^{b}Rabbit pAb
^{c}Disease-distinguishing

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VIII. REFERENCES

The following references, to the extent that they provide exemplary procedural or other details supplementary to those set forth herein, are specifically incorporated herein by reference:
Remington's Pharmaceutical Sciences, 15th Edition.
U.S. Pat. No. 3,817,837
U.S. Pat. No. 3,850,752
U.S. Pat. No. 3,939,350
U.S. Pat. No. 3,996,345
U.S. Pat. No. 4,196,265
U.S. Pat. No. 4,275,149
U.S. Pat. No. 4,277,437
U.S. Pat. No. 4,366,241
U.S. Pat. No. 4,472,509
U.S. Pat. No. 4,554,101
U.S. Pat. No. 4,680,338
U.S. Pat. No. 4,816,567
U.S. Pat. No. 4,867,973
U.S. Pat. No. 4,938,948
U.S. Pat. No. 5,021,236
U.S. Pat. No. 5,141,648
U.S. Pat. No. 5,196,066
U.S. Pat. No. 5,563,250
U.S. Pat. No. 5,565,332
U.S. Pat. No. 5,856,456
U.S. Pat. No. 5,880,270
U.S. Pat. No. 6,485,982
“Antibodies: A Laboratory Manual,” Cold Spring Harbor Press, Cold Spring Harbor, N.Y., 1988.
Abbondanzo et al., Am. J. Pediatr. Hematol. Oncol., 12(4), 480-489, 1990.
Alonso et al., Nat. Med., 2:783-787, 1996.
Allred et al., Arch. Surg., 125(1), 107-113, 1990.
Arriagada et al., Neurology, 42:631-639, 1992.
Atherton et al., Biol. of Reproduction, 32, 155-171, 1985.
Barzon et al., Euro Surveill. 2016 Aug. 11; 21(32).
Beltramello et al., Cell Host Microbe 8, 271-283, 2010.
Bramblett et al., Neuron., 10:1089-1099, 1993.
Brown et al., J. Immunol. Meth., 12; 130(1), :111-121, 1990.
Campbell, In: Monoclonal Antibody Technology, Laboratory Techniques in Biochemistry and Molecular Biology, Vol. 13, Burden and Von Knippenberg, Eds. pp. 75-83, Amsterdam, Elsevier, 1984.
Capaldi et al., Biochem. Biophys. Res. Comm., 74(2):425-433, 1977.
Chen et al., Nat Commun. 10(1):2493. doi: 10.1038/s41467-019-10355-1, 2019.
De Jager et al., Semin. Nucl. Med. 23(2), 165-179, 1993.
Dholakia et al., J. Biol. Chem., 264, 20638-20642, 1989.
Diamond et al., J Virol 77, 2578-2586, 2003.
Doolittle and Ben-Zeev, Methods Mol. Biol., 109, :215-237, 1999.
Duffy et al., N. Engl. J. Med. 360, 2536-2543, 2009.
Elder et al. Infections, infertility and assisted reproduction. Part II: Infections in reproductive medicine & Part III: Infections and the assisted reproductive laboratory. Cambridge UK: Cambridge University Press; 2005.
Gefter et al., Somatic Cell Genet., 3:231-236, 1977.
Gornet et al., Semin Reprod Med. 2016 September; 34(5):285-292. Epub 2016 Sep. 14.
Gulbis and Galand, Hum. Pathol. 24(12), 1271-1285, 1993.
Gustke et al., FEBS Lett., 307:199-205, 1992.
Halfon et al., PLoS ONE 2010; 5 (5) e10569.
Hartig et al., Eur. J. Neurosci., 25:69-80, 2007.
Hessell et al., Nature 449, 101-4, 2007.
Hutton et al., Nature, 393:702-705, 1998.
Khatoon et al., Ann. of Neurology, 26, 210-219, 1989.
King et al., J. Biol. Chem., 269, 10210-10218, 1989.
Kohler and Milstein, Eur. J. Immunol., 6, 511-519, 1976.
Kohler and Milstein, Nature, 256, 495-497, 1975.
Kyte and Doolittle, J. Mol. Biol., 157(1):105-132, 1982.
Mansuy et al., Lancet Infect Dis. 2016 October; 16(10):1106-7.
Mirbaha et al., Elife, pii: e36584. doi: 10.7554/eLife.36584, 2018.
Nakamura et al., In: Enzyme Immunoassays: Heterogeneous and Homogeneous Systems, Chapter 27, 1987.
O'Shannessy et al., J. Immun. Meth., 99, 153-161, 1987.
Persic et al., Gene 187:1, 1997
Poorkaj et al., Ann. Neurol., 43:815-825, 1998.
Potter and Haley, Meth. Enzymol., 91, 613-633, 1983.
Purpura et al., Lancet Infect Dis. 2016 October; 16(10):1107-8. Epub 2016 Sep. 19.
Remington's Pharmaceutical Sciences, 15th Ed., 3:624-652, 1990.
Spillantini et al., Proc. Natl. Acad. Sci. USA, 95:7737-7741, 1998.
Tang et al., J. Biol. Chem., 271:28324-28330, 1996.
Wawrzynczak & Thorpe, In: Immunoconjugates, Antibody Conjugates In Radioimaging And Therapy Of Cancer, Vogel (Ed.), NY, Oxford University Press, 28, 1987.
Yu et al., J Immunol Methods 336, 142-151, doi:10.1016/j.jim.2008.04.008, 2008.

1. A method of treating a subject with or at risk of developing a tauopathy comprising administering to said subject one or more peptides from Tables B or C.

2. The method of claim 1, further comprising administering the same or a different peptide from Tables B or C at a second time point.

3. The method of claim 1, wherein said subject suffers from Alzheimer's Disease, Progressive Supranuclear Palsy, Corticobasal Degeneration, Frontotemporal Dementia, Multiple Sclerosis, Argyrophilic Grain Disease, Chronic Traumatic Encephalopathy, or Primary Age-Related Tauopathy.

4. The method of claim 1, further comprising administering to said subject an adjuvant and/or a biological response modifier.

5. The method of claim 1, wherein said subject has been diagnosed with a tauopathy.

6. The method of claim 1, wherein said subject is human subject.

7. The method of claim 1, wherein said subject is non-human mammalian subject.

8. The method of claim 1, wherein administering comprises intramuscular injection, oral delivery, subcutaneous injection, transdermal delivery, inhalation, intravenous injection, intrathecal or intraventricular injection.

9. The method of claim 1, wherein said peptide is no more than about 100 residues in length, no more than about 50 residues in length, or no more than about 38 residues in length.

10. The method of claim 1, wherein said peptide is at least 5 residues in length, is at least 6 residues in length, or is at least 10 residues in length.

11. A method of treating a subject with or at risk of developing a tauopathy comprising administering to said subject one or more antibodies or antibody fragments that bind to a tau epitope defined by one or more peptides from Tables B or C.

12-21. (canceled)

21. A peptide vaccine comprising one or more peptides from Tables B or C in a pharmaceutically acceptable diluent, buffer or excipient.

22. The vaccine of claim 21, further comprising an adjuvant and/or a biological response modifier.

23. The vaccine of claim 21, wherein said vaccine is formulated for intramuscular injection, oral delivery, subcutaneous injection, transdermal delivery, inhalation, intravenous injection, intrathecal or intraventricular injection.

24. The vaccine of claim 21, wherein said peptide is no more than about 100 residues in length, no more than about 50 residues in length, or no more than about 37 residues in length.

25. The vaccine of claim 21, wherein said peptide is at least 5 residues in length, is at least 10 residues in length, or is at least 16 residues in length.

26. A vaccine comprising one or more antibodies or antibody fragments that bind to a tau epitope defined by one or more peptides from Tables B or C.

27-30. (canceled)

31. A method of detecting tau protein or a structurally related antigen in a subject comprising:
(a) contacting a sample from said subject with one more more antibodies or antibody fragments that bind to a tau epitope defined by one or more peptides from from Tables B or C; and
(b) detecting said tau protein or structurally related antigen in said sample by binding of said one or more antibodies or antibody fragments to said tau protein or structurally related antigen in said sample.

32-40. (canceled)


SEQUENCE LISTING (151 sequences)

SEQ ID NO: 1 - protein, 6 aa, Artificial Sequence
  Synthetic peptide
VQIVYK

SEQ ID NO: 2 - protein, 6 aa, Artificial Sequence
  Synthetic peptide
DNIKHV

SEQ ID NO: 3 - protein, 6 aa, Artificial Sequence
  Synthetic peptide
VQIINK

SEQ ID NO: 4 - protein, 17 aa, Artificial Sequence
  Synthetic peptide
DNIKHVPGGGSVQIVYK

SEQ ID NO: 5 - protein, 17 aa, Artificial Sequence
  Synthetic peptide
DNIKHVLGGGSVQIVYK

SEQ ID NO: 6 - protein, 17 aa, Artificial Sequence
  Synthetic peptide
DNIKHVSGGGSVQIVYK

SEQ ID NO: 7 - protein, 17 aa, Artificial Sequence
  Synthetic peptide
  Fluorinated 4S-F-Proline; cis (7)..(7)
DNIKHVXGGGSVQIVYK

SEQ ID NO: 8 - protein, 17 aa, Artificial Sequence
  Synthetic peptide
  Fluorinated 4R-F-Proline; trans (7)..(7)
DNIKHVXGGGSVQIVYK

SEQ ID NO: 9 - protein, 11 aa, Artificial Sequence
  Synthetic peptide
DNIKHVPGGGS

SEQ ID NO: 10 - protein, 11 aa, Artificial Sequence
  Synthetic peptide
DNIKHVLGGGS

SEQ ID NO: 11 - protein, 11 aa, Artificial Sequence
  Synthetic peptide
DNIKHVSGGGS

SEQ ID NO: 12 - protein, 11 aa, Artificial Sequence
  Synthetic peptide
  Fluorinated 4S-F-Proline; cis (7)..(7)
DNIKHVXGGGS

SEQ ID NO: 13 - protein, 11 aa, Artificial Sequence
  Synthetic peptide
  Fluorinated 4R-F-Proline; trans (7)..(7)
DNIKHVXGGGS

SEQ ID NO: 14 - protein, 12 aa, Artificial Sequence
  Synthetic peptide
VPGGGSVQIVYK

SEQ ID NO: 15 - protein, 12 aa, Artificial Sequence
  Synthetic peptide
VLGGGSVQIVYK

SEQ ID NO: 16 - protein, 12 aa, Artificial Sequence
  Synthetic peptide
VSGGGSVQIVYK

SEQ ID NO: 17 - protein, 12 aa, Artificial Sequence
  Synthetic peptide
  Fluorinated 4S-F-Proline; cis (2)..(2)
VXGGGSVQIVYK

SEQ ID NO: 18 - protein, 12 aa, Artificial Sequence
  Synthetic peptide
  Fluorinated 4R-F-Proline; trans (2)..(2)
VXGGGSVQIVYK

SEQ ID NO: 19 - protein, 19 aa, Artificial Sequence
  Synthetic peptide
TENLKHQPGGGKVQIINKK

SEQ ID NO: 20 - protein, 19 aa, Artificial Sequence
  Synthetic peptide
TENLKHQLGGGKVQIINKK

SEQ ID NO: 21 - protein, 19 aa, Artificial Sequence
  Synthetic peptide
TENLKHQSGGGKVQIINKK

SEQ ID NO: 22 - protein, 19 aa, Artificial Sequence
  Synthetic peptide
  Fluorinated 4S-F-Proline; cis (8)..(8)
TENLKHQXGGGKVQIINKK

SEQ ID NO: 23 - protein, 19 aa, Artificial Sequence
  Synthetic peptide
  Fluorinated 4R-F-Proline; trans (8)..(8)
TENLKHQXGGGKVQIINKK

SEQ ID NO: 24 - protein, 24 aa, Artificial Sequence
  Synthetic peptide
  Phosphoserine (5)..(5)
SKIGXTENLKHQPGGGKVQIINKK

SEQ ID NO: 25 - protein, 24 aa, Artificial Sequence
  Synthetic peptide
  Phosphoserine (5)..(5)
SKIGXTENLKHQLGGGKVQIINKK

SEQ ID NO: 26 - protein, 24 aa, Artificial Sequence
  Synthetic peptide
  Phosphoserine (5)..(5)
SKIGXTENLKHQSGGGKVQIINKK

SEQ ID NO: 27 - protein, 24 aa, Artificial Sequence
  Synthetic peptide
  Phosphoserine (5)..(5)
  Fluorinated 4S-F-Proline cis (13)..(13)
SKIGXTENLKHQXGGGKVQIINKK

SEQ ID NO: 28 - protein, 24 aa, Artificial Sequence
  Synthetic peptide
  Phosphoserine (5)..(5)
  Fluorinated 4R-F-Proline trans (13)..(13)
SKIGXTENLKHQXGGGKVQIINKK

SEQ ID NO: 29 - protein, 12 aa, Artificial Sequence
  Synthetic peptide
TENLKHQPGGGK

SEQ ID NO: 30 - protein, 12 aa, Artificial Sequence
  Synthetic peptide
TENLKHQLGGGK

SEQ ID NO: 31 - protein, 12 aa, Artificial Sequence
  Synthetic peptide
TENLKHQSGGGK

SEQ ID NO: 32 - protein, 12 aa, Artificial Sequence
  Synthetic peptide
  Fluorinated 4S-F-Proline; cis (8)..(8)
TENLKHQXGGGK

SEQ ID NO: 33 - protein, 12 aa, Artificial Sequence
  Synthetic peptide
  Fluorinated 4R-F-Proline; trans (8)..(8)
TENLKHQXGGGK

SEQ ID NO: 34 - protein, 17 aa, Artificial Sequence
  Synthetic peptide
  Phosphoserine (5)..(5)
SKIGXTENLKHQPGGGK

SEQ ID NO: 35 - protein, 17 aa, Artificial Sequence
  Synthetic peptide
  Phosphoserine (5)..(5)
SKIGXTENLKHQLGGGK

SEQ ID NO: 36 - protein, 17 aa, Artificial Sequence
  Synthetic peptide
  Phosphoserine (5)..(5)
SKIGXTENLKHQSGGGK

SEQ ID NO: 37 - protein, 17 aa, Artificial Sequence
  Synthetic peptide
  Phosphoserine (5)..(5)
  Fluorinated 4S-F-Proline cis (13)..(13)
SKIGXTENLKHQXGGGK

SEQ ID NO: 38 - protein, 17 aa, Artificial Sequence
  Synthetic peptide
  Phosphoserine (5)..(5)
  Fluorinated 4R-F-Proline trans (13)..(13)
SKIGXTENLKHQXGGGK

SEQ ID NO: 39 - protein, 13 aa, Artificial Sequence
  Synthetic peptide
QPGGGKVQIINKK

SEQ ID NO: 40 - protein, 13 aa, Artificial Sequence
  Synthetic peptide
QLGGGKVQIINKK

SEQ ID NO: 41 - protein, 13 aa, Artificial Sequence
  Synthetic peptide
QSGGGKVQIINKK

SEQ ID NO: 42 - protein, 13 aa, Artificial Sequence
  Synthetic peptide
  Fluorinated 4S-F-Proline; cis (2)..(2)
QXGGGKVQIINKK

SEQ ID NO: 43 - protein, 13 aa, Artificial Sequence
  Synthetic peptide
  Fluorinated 4R-F-Proline; trans (2)..(2)
QXGGGKVQIINKK

SEQ ID NO: 44 - protein, 18 aa, Artificial Sequence
  Synthetic peptide
GNIHHKPGGGQVEVKSEK

SEQ ID NO: 45 - protein, 18 aa, Artificial Sequence
  Synthetic peptide
GNIHHKLGGGQVEVKSEK

SEQ ID NO: 46 - protein, 18 aa, Artificial Sequence
  Synthetic peptide
GNIHHKSGGGQVEVKSEK

SEQ ID NO: 47 - protein, 18 aa, Artificial Sequence
  Synthetic peptide
  Fluorinated 4S-F-Proline; cis (7)..(7)
GNIHHKXGGGQVEVKSEK

SEQ ID NO: 48 - protein, 18 aa, Artificial Sequence
  Synthetic peptide
  Fluorinated 4R-F-Proline; trans (7)..(7)
GNIHHKXGGGQVEVKSEK

SEQ ID NO: 49 - protein, 11 aa, Artificial Sequence
  Synthetic peptide
GNIHHKPGGGQ

SEQ ID NO: 50 - protein, 11 aa, Artificial Sequence
  Synthetic peptide
GNIHHKLGGGQ

SEQ ID NO: 51 - protein, 11 aa, Artificial Sequence
  Synthetic peptide
GNIHHKSGGGQ

SEQ ID NO: 52 - protein, 11 aa, Artificial Sequence
  Synthetic peptide
  Fluorinated 4S-F-Proline; cis (7)..(7)
GNIHHKXGGGQ

SEQ ID NO: 53 - protein, 11 aa, Artificial Sequence
  Synthetic peptide
  Fluorinated 4R-F-Proline; trans (7)..(7)
GNIHHKXGGGQ

SEQ ID NO: 54 - protein, 13 aa, Artificial Sequence
  Synthetic peptide
KPGGGQVEVKSEK

SEQ ID NO: 55 - protein, 13 aa, Artificial Sequence
  Synthetic peptide
KLGGGQVEVKSEK

SEQ ID NO: 56 - protein, 13 aa, Artificial Sequence
  Synthetic peptide
KSGGGQVEVKSEK

SEQ ID NO: 57 - protein, 13 aa, Artificial Sequence
  Synthetic peptide
  Fluorinated 4S-F-Proline; cis (2)..(2)
KXGGGQVEVKSEK

SEQ ID NO: 58 - protein, 13 aa, Artificial Sequence
  Synthetic peptide
  Fluorinated 4R-F-Proline; trans (2)..(2)
KXGGGQVEVKSEK

SEQ ID NO: 59 - protein, 18 aa, Artificial Sequence
  Synthetic peptide
DNITHVPGGGNKKIETHK

SEQ ID NO: 60 - protein, 18 aa, Artificial Sequence
  Synthetic peptide
DNITHVLGGGNKKIETHK

SEQ ID NO: 61 - protein, 18 aa, Artificial Sequence
  Synthetic peptide
DNITHVSGGGNKKIETHK

SEQ ID NO: 62 - protein, 18 aa, Artificial Sequence
  Synthetic peptide
  Fluorinated 4S-F-Proline; cis (7)..(7)
DNITHVXGGGNKKIETHK

SEQ ID NO: 63 - protein, 18 aa, Artificial Sequence
  Synthetic peptide
  Fluorinated 4R-F-Proline; trans (7)..(7)
DNITHVXGGGNKKIETHK

SEQ ID NO: 64 - protein, 11 aa, Artificial Sequence
  Synthetic peptide
DNITHVPGGGN

SEQ ID NO: 65 - protein, 11 aa, Artificial Sequence
  Synthetic peptide
DNITHVLGGGN

SEQ ID NO: 66 - protein, 11 aa, Artificial Sequence
  Synthetic peptide
DNITHVSGGGN

SEQ ID NO: 67 - protein, 11 aa, Artificial Sequence
  Synthetic peptide
  Fluorinated 4S-F-Proline; cis (7)..(7)
DNITHVXGGGN

SEQ ID NO: 68 - protein, 11 aa, Artificial Sequence
  Synthetic peptide
  Fluorinated 4R-F-Proline; trans (7)..(7)
DNITHVXGGGN

SEQ ID NO: 69 - protein, 13 aa, Artificial Sequence
  Synthetic peptide
VPGGGNKKIETHK

SEQ ID NO: 70 - protein, 13 aa, Artificial Sequence
  Synthetic peptide
VLGGGNKKIETHK

SEQ ID NO: 71 - protein, 13 aa, Artificial Sequence
  Synthetic peptide
VSGGGNKKIETHK

SEQ ID NO: 72 - protein, 13 aa, Artificial Sequence
  Synthetic peptide
  Fluorinated 4S-F-Proline; cis (2)..(2)
VXGGGNKKIETHK

SEQ ID NO: 73 - protein, 13 aa, Artificial Sequence
  Synthetic peptide
  Fluorinated 4R-F-Proline; trans (2)..(2)
VXGGGNKKIETHK

SEQ ID NO: 74 - protein, 20 aa, Artificial Sequence
  Synthetic peptide
LQTAPVPMPDLKNVKSKIGS

SEQ ID NO: 75 - protein, 10 aa, Artificial Sequence
  Synthetic peptide
LKNVKSKIGS

SEQ ID NO: 76 - protein, 10 aa, Artificial Sequence
  Synthetic peptide
LQTAPVPMPD

SEQ ID NO: 77 - protein, 14 aa, Artificial Sequence
  Synthetic peptide
KLDLSNVQSKCGSK

SEQ ID NO: 78 - protein, 7 aa, Artificial Sequence
  Synthetic peptide
QSKCGSK

SEQ ID NO: 79 - protein, 7 aa, Artificial Sequence
  Synthetic peptide
KLDLSNV

SEQ ID NO: 80 - protein, 14 aa, Artificial Sequence
  Synthetic peptide
PVDLSKVTSKCGSL

SEQ ID NO: 81 - protein, 7 aa, Artificial Sequence
  Synthetic peptide
TSKCGSL

SEQ ID NO: 82 - protein, 7 aa, Artificial Sequence
  Synthetic peptide
PVDLSKV

SEQ ID NO: 83 - protein, 14 aa, Artificial Sequence
  Synthetic peptide
LDFKDRVQSKIGSL

SEQ ID NO: 84 - protein, 7 aa, Artificial Sequence
  Synthetic peptide
QSKIGSL

SEQ ID NO: 85 - protein, 7 aa, Artificial Sequence
  Synthetic peptide
LDFKDRV

SEQ ID NO: 86 - protein, 20 aa, Artificial Sequence
  Synthetic peptide
LTFRENAKAKTDHGAEIVYK

SEQ ID NO: 87 - protein, 11 aa, Artificial Sequence
  Synthetic peptide
KTDHGAEIVYK

SEQ ID NO: 88 - protein, 9 aa, Artificial Sequence
  Synthetic peptide
LTFRENAKA

SEQ ID NO: 89 - protein, 15 aa, Artificial Sequence
  Synthetic peptide
KVQIINKKLDLSNVQ

SEQ ID NO: 90 - protein, 12 aa, Artificial Sequence
  Synthetic peptide
SKCGSKDNIKHV

SEQ ID NO: 91 - protein, 16 aa, Artificial Sequence
  Synthetic peptide
SVQIVYKPVDLSKVTS

SEQ ID NO: 92 - protein, 7 aa, Artificial Sequence
  Synthetic peptide
KVQIINK

SEQ ID NO: 93 - protein, 18 aa, Artificial Sequence
  Synthetic peptide
KVQIINKKLDLSNVQSKC

SEQ ID NO: 94 - protein, 21 aa, Artificial Sequence
  Synthetic peptide
KVQIINKKLDLSNVQSKCGSK

SEQ ID NO: 95 - protein, 27 aa, Artificial Sequence
  Synthetic peptide
KVQIINKKLDLSNVQSKCGSKDNIKHV

SEQ ID NO: 96 - protein, 32 aa, Artificial Sequence
  Synthetic peptide
KVQIINKKLDLSNVQSKCGSKDNIKHVPGGGS

SEQ ID NO: 97 - protein, 31 aa, Artificial Sequence
  Synthetic peptide
KLDLSNVQSKCGSKDNIKHVPGGGSVQIVYK

SEQ ID NO: 98 - protein, 21 aa, Artificial Sequence
  Synthetic peptide
CGSKDNIKHVPGGGSVQIVYK

SEQ ID NO: 99 - protein, 11 aa, Artificial Sequence
  Synthetic peptide
PGGGSVQIVYK

SEQ ID NO: 100 - protein, 30 aa, Artificial Sequence
  Synthetic peptide
KLDLSNVQSKCGSKDNIKHVLGGGSVQIVY

SEQ ID NO: 101 - protein, 21 aa, Artificial Sequence
  Synthetic peptide
CGSKDNIKHVLGGGSVQIVYK

SEQ ID NO: 102 - protein, 31 aa, Artificial Sequence
  Synthetic peptide
KLDLSNVQSKCGSKDNIKHVSGGGSVQIVYK

SEQ ID NO: 103 - protein, 21 aa, Artificial Sequence
  Synthetic peptide
CGSKDNIKHVSGGGSVQIVYK

SEQ ID NO: 104 - protein, 31 aa, Artificial Sequence
  Synthetic peptide
  Fluorinated 4S-F-Proline; cis (21)..(21)
KLDLSNVQSKCGSKDNIKHVXGGGSVQIVYK

SEQ ID NO: 105 - protein, 31 aa, Artificial Sequence; identical to SEQ ID NO: 104
  Synthetic peptide
  Fluorinated 4R-F-Proline; trans (21)..(21)

SEQ ID NO: 106 - protein, 38 aa, Artificial Sequence
  Synthetic peptide
KVQIINKKLDLSNVQSKCGSKDNIKHVLGGGSVQIVYK

SEQ ID NO: 107 - protein, 38 aa, Artificial Sequence
  Synthetic peptide
KVQIINKKLDLSNVQSKCGSKDNIKHVSGGGSVQIVYK

SEQ ID NO: 108 - protein, 38 aa, Artificial Sequence
  Synthetic peptide
  Fluorinated 4S-F-Proline; cis (28)..(28)
KVQIINKKLDLSNVQSKCGSKDNIKHVXGGGSVQIVYK

SEQ ID NO: 109 - protein, 38 aa, Artificial Sequence; identical to SEQ ID NO: 108
  Synthetic peptide
  Fluorinated 4R-F-Proline; trans (28)..(28)

SEQ ID NO: 110 - protein, 18 aa, Artificial Sequence
  Synthetic peptide
TENLKHQPGGGKVQIVYK

SEQ ID NO: 111 - protein, 17 aa, Artificial Sequence
  Synthetic peptide
YGLGDRKDQGGYTMHQD

SEQ ID NO: 112 - protein, 13 aa, Artificial Sequence
  Synthetic peptide
DRKDQGGYTMHQD

SEQ ID NO: 113 - protein, 16 aa, Artificial Sequence
  Synthetic peptide
RKDQGGYTMHQDQEGD

SEQ ID NO: 114 - protein, 11 aa, Artificial Sequence
  Synthetic peptide
SKDGTGSDDKK

SEQ ID NO: 115 - protein, 31 aa, Artificial Sequence
  Synthetic peptide
KSKDGTGSDDKKAKGADGKTKIATPRGAAPP

SEQ ID NO: 116 - protein, 19 aa, Artificial Sequence
  Synthetic peptide
TENLKHQPGGGKVQIINKK

SEQ ID NO: 117 - protein, 19 aa, Artificial Sequence
  Synthetic peptide
VQIINKKLDLSNVQSKCGS

SEQ ID NO: 118 - protein, 18 aa, Artificial Sequence
  Synthetic peptide
KDNIKHVPGGGSVQIVYK

SEQ ID NO: 119 - protein, 18 aa, Artificial Sequence
  Synthetic peptide
GNIHHKPGGGQVEVKSEK

SEQ ID NO: 120 - protein, 18 aa, Artificial Sequence
  Synthetic peptide
DNITHVPGGGNKKIETHK

SEQ ID NO: 121 - protein, 18 aa, Artificial Sequence
  Synthetic peptide
TENLKHQPGGGKVQIVYK

SEQ ID NO: 122 - protein, 18 aa, Artificial Sequence
  Synthetic peptide
TENLKHQLGGGKVQIVYK

SEQ ID NO: 123 - protein, 18 aa, Artificial Sequence
  Synthetic peptide
TENLKHQSGGGKVQIVYK

SEQ ID NO: 124 - protein, 18 aa, Artificial Sequence
  Synthetic peptide
  Fluorinated 4S-F-Proline cis (8)..(8)
TENLKHQXGGGKVQIVYK

SEQ ID NO: 125 - protein, 18 aa, Artificial Sequence
  Synthetic peptide
  Fluorinated 4R-F-Proline trans (8)..(8)
TENLKHQXGGGKVQIVYK

SEQ ID NO: 126 - protein, 23 aa, Artificial Sequence
  Synthetic peptide
  Phosphoserine (5)..(5)
SKIGXTENLKHQPGGGKVQIVYK

SEQ ID NO: 127 - protein, 23 aa, Artificial Sequence
  Synthetic peptide
  Phosphoserine (5)..(5)
SKIGXTENLKHQLGGGKVQIVYK

SEQ ID NO: 128 - protein, 23 aa, Artificial Sequence
  Synthetic peptide
  Phosphoserine (5)..(5)
SKIGXTENLKHQSGGGKVQIVYK

SEQ ID NO: 129 - protein, 23 aa, Artificial Sequence
  Synthetic peptide
  Phosphoserine (5)..(5)
  Fluorinated 4S-F-Proline cis (13)..(13)
SKIGXTENLKHQXGGGKVQIVYK

SEQ ID NO: 130 - protein, 23 aa, Artificial Sequence
  Synthetic peptide
  Phosphoserine (5)..(5)
  Fluorinated 4R-F-Proline trans (13)..(13)
SKIGXTENLKHQXGGGKVQIVYK

SEQ ID NO: 131 - protein, 12 aa, Artificial Sequence
  Synthetic peptide
TENLKHQPGGGK

SEQ ID NO: 132 - protein, 12 aa, Artificial Sequence
  Synthetic peptide
TENLKHQLGGGK

SEQ ID NO: 133 - protein, 12 aa, Artificial Sequence
  Synthetic peptide
TENLKHQSGGGK

SEQ ID NO: 134 - protein, 12 aa, Artificial Sequence
  Synthetic peptide
  Fluorinated 4S-F-Proline cis (8)..(8)
TENLKHQXGGGK

SEQ ID NO: 135 - protein, 12 aa, Artificial Sequence
  Synthetic peptide
  Fluorinated 4R-F-Proline trans (8)..(8)
TENLKHQXGGGK

SEQ ID NO: 136 - protein, 17 aa, Artificial Sequence
  Synthetic peptide
  Phosphoserine (5)..(5)
SKIGXTENLKHQPGGGK

SEQ ID NO: 137 - protein, 17 aa, Artificial Sequence
  Synthetic peptide
  Phosphoserine (5)..(5)
SKIGXTENLKHQLGGGK

SEQ ID NO: 138 - protein, 17 aa, Artificial Sequence
  Synthetic peptide
  Phosphoserine (5)..(5)
SKIGXTENLKHQSGGGK

SEQ ID NO: 139 - protein, 17 aa, Artificial Sequence
  Synthetic peptide
  Phosphoserine (5)..(5)
  Fluorinated 4S-F-Proline cis (13)..(13)
SKIGXTENLKHQXGGGK

SEQ ID NO: 140 - protein, 17 aa, Artificial Sequence
  Synthetic peptide
  Phosphoserine (5)..(5)
  Fluorinated 4R-F-Proline trans (13)..(13)
SKIGXTENLKHQXGGGK

SEQ ID NO: 141 - protein, 12 aa, Artificial Sequence
  Synthetic peptide
QPGGGKVQIVYK

SEQ ID NO: 142 - protein, 12 aa, Artificial Sequence
  Synthetic peptide
QLGGGKVQIVYK

SEQ ID NO: 143 - protein, 12 aa, Artificial Sequence
  Synthetic peptide
QSGGGKVQIVYK

SEQ ID NO: 144 - protein, 12 aa, Artificial Sequence
  Synthetic peptide
  Fluorinated 4S-F-Proline cis (2)..(2)
QXGGGKVQIVYK

SEQ ID NO: 145 - protein, 12 aa, Artificial Sequence
  Synthetic peptide
  Fluorinated 4R-F-Proline trans (2)..(2)
QXGGGKVQIVYK

SEQ ID NO: 146 - protein, 18 aa, Artificial Sequence
  Synthetic peptide
TENLKHQPGGGKVQIINK

SEQ ID NO: 147 - protein, 18 aa, Artificial Sequence
  Synthetic peptide
KDNIKHVPGGGSVQIVYK

SEQ ID NO: 148 - protein, 19 aa, Artificial Sequence
  Synthetic peptide
LGNIHHKPGGGQVEVKSEK

SEQ ID NO: 149 - protein, 18 aa, Artificial Sequence
  Synthetic peptide
LDNITHVPGGGNKKIETH

SEQ ID NO: 150 - protein, 12 aa, Artificial Sequence
  Synthetic peptide
TPPKSPSSAKSR

SEQ ID NO: 151 - protein, 19 aa, Artificial Sequence
  Synthetic peptide
SSAKSRLQTAPVPMPDLKN